(19) United States
(12) Patent Application Publication
(10) Pub. No.: US 2024/0309149 A1
(43) Pub. Date: Sep. 19, 2024

(54) POLY(DIALKYL BETA-LACTONE) COMPOSITIONS, METHODS OF MAKING SAME AND USES THEREOF

(71) Applicant: CORNELL UNIVERSITY, Ithaca, NY (US)

(72) Inventors: Geoffrey W. Coates, Lansing, NY (US); Anne M. LaPointe, Ithaca, NY (US); Zhiyao Zhou, Ithaca, NY (US)

(21) Appl. No.: 18/577,985

(22) PCT Filed: Jul. 9, 2022
(86) PCT No.: PCT/US2022/036604

§ 371 Date: Jan. 9, 2024

Related U.S. Application Data
(60) Provisional application No. 63/220,301, filed on Jul. 9, 2021.

(51) Int. Cl. C08G 63/08; C07C 51/09; C08G 63/82

(52) U.S. Cl. CPC C08G 63/08; C07C 51/09; C08G 63/823; C08G 2230/00

(57) ABSTRACT

Poly(dialkyl beta-lactone) (PDABL) compositions, methods of making same, and uses thereof. In various examples, the PDABL compositions are homopolymers or copolymers. The PDABL compositions comprise a plurality of dialkyl P-lactone (DABL) repeat units. The (PDABL) compositions can be formed by ring-opening polymerization of dialkyl-P-lactone(s). PDABL composition(s) can be used to form various articles of manufacture, such as, for example, packaging articles, single-use articles, sports articles, bio-medical articles, agricultural articles, automotive articles, electronic articles, and the like. In various examples, the PDABL composition or an article of manufacture is biodegradable.

### CROSS-REFERENCE TO RELATED APPLICATIONS

This application claims the benefit of U.S. Provisional Application No. 63/220,301 filed Jul. 9, 2021. The entire contents of the above-identified application are hereby fully incorporated herein by reference.

### BACKGROUND OF THE DISCLOSURE

Polyolefin plastics are widely used due to their low cost and outstanding properties, but their environmental persistence presents a major societal challenge. Polyhydroxyalkanoates, often produced by bacteria as carbon- and energy-storage materials, can be biodegradable substitutes for polyolefins due to their comparable thermal properties. However, their applications are limited owing to low production volumes and unsatisfactory processibility.

In the year 2020, the cumulative global mass of synthetic plastics achieved the dubious distinction of exceeding the total combined mass of all terrestrial and marine animals. As an inevitable consequence, the ever-growing amount of plastic waste has become a major threat to the planet, despite governmental action to mitigate plastic pollution. While mechanical recycling provides a means to collect and sort postconsumer plastics, alternative strategies, such as biodegradation, are needed to tackle plastic wastes that leak into the environment, especially the most common yet environmentally persistent polyolefins, with an estimated 4.8-12.7 million metric tons of total plastics entering the ocean in 2010.

In this regard, the biodegradable polyhydroxyalkanoates (PHA) produced by bacteria continue to attract industrial attention as potential substitutes for polyolefin plastics in packaging and other single-use applications. Polylactic acid (PLA) is widely used in single-use applications such as disposable cups and cutlery, but its low T_{g} limits its use in higher temperature applications. Polypivalolactone has extremely high Tₘ (245° C.) and T_{d} (445° C.), but its industrial production is currently cost-prohibitive. The enantiopure, isotactic biopolymer R-poly(hydroxybutyrate) (R-PHB), which is produced by bacteria, is an attractive target due to its high melting point (ca 175° C.), biodegradability and low toxicity. However, it suffers from poor processability and the homopolymers are highly brittle. The processibility of PHB may be improved by addition of small amounts of comonomers with longer side chains. However, its narrow processing window severely limits its applications.

PHB may also be produced via chemical synthesis. It exhibits low toxicity and a high melting point (ca. 175° C.) that is similar to isotactic polypropylene (iPP) but the low production volumes, brittleness and thermal instability of R-P3HB homopolymers have limited their widespread use. Although the mechanical properties of R-P3HB may be improved by copolymerizing with other monomers, or in combination with more elaborate stereochemical and sequence control, other problems have not been fully addressed. No PHA polymers have been reported that meet the polyolefin-like thermal and mechanical properties while maintaining their recyclability, industrial practicality, and economic viability. Thus, there exists an ongoing and unmet need for improved PHA polymers.

### SUMMARY OF THE DISCLOSURE

In various examples, the poly(dialkyl β-lactone) (PDABL) composition comprises one or more dialkyl β-lactone (DABL) repeat unit(s) (e.g.,

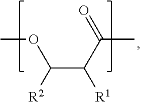

where R¹ and R² are each, independently at each occurrence, an alkyl group. In various examples, a PDABL composition is a homopolymer or a copolymer. In various examples, the PDABL composition comprises one or more polymeric chain(s) and/or one or more oligomeric chain(s) comprising one or more α,β-dialkyl β-lactone (DABL) repeat unit(s). In various examples, the PDABL composition comprises one or more or all of the following: a molecular weight (Mw and/or Mn) of from about 50 kiloDalton (kD) or greater, from about 60 kD or greater, from about 80 kD or greater, from about 100 kD or greater, or from about 150 kD or greater; one or more crystalline domain(s); a combination of cis and trans DABL repeat units; at least partially or all atactic DABL repeat units, or at least partially or all syndiotactic DABL repeat units, or any combination thereof; one or more non-DABL repeat units; or two or more DABL repeat units with different R¹ groups, R² groups. In various examples, the PDABL composition comprises one or more polymeric chain(s) and/or one or more oligomeric chain(s) comprising one or more α,β-dialkyl β-lactone (DABL) repeat unit(s) having the following structure:

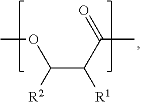

where R¹ and R² are each a methyl group, and where the polymeric and/or the oligomeric chain(s) is/are not completely trans isotactic. In various examples, the polymeric and/or the oligomeric chain(s) has/have a weight average molecular weight (M_{w}) or a number average molecular weight (Mₙ) of at least about 25 kilodalton (kD) to about 500 kD (e.g., 75 kilodalton (kD) to about 300 kD). In various examples, the polymeric and/or the oligomeric chain(s) has/have a polydispersity index (M_{w}/Mₙ) of about 1 to about 10 (e.g., about 1 to about 1.5). In various examples, at least a portion of or all of the polymeric and/or the oligomeric chain(s), independently, at least partially or completely, comprise(s) cis and/or trans DABL repeat units. In various examples, at least a portion of or all of the polymeric and/or the oligomeric chain(s), independently, at least partially or completely, comprise(s) a molar ratio of cis to trans DABL repeat units of about 1:99 to about 99:1 (e.g., about 70:1 to about 99:1). In various examples, at least a portion of or all of the polymeric and/or the oligomeric chain(s), independently, at least partially or completely, comprise(s) randomly oriented and/or stereoregular DABL diads. In various examples, the stereoregular DABL diads comprise meso ([m]) and/or racemo ([r]) DABL diads. In various examples, at least a portion of or all of the polymeric and/or the oligomeric chain(s), independently, at least partially or completely, comprise(s) trans random, cis random, trans meso ([m]), cis meso ([m]), trans racemo ([r]), or cis racemo ([r]) DABL diads, or any combination thereof. In various examples, at least a portion of the polymeric and/or the oligomeric chain(s), independently, comprise(s) 95 mol % or less of any one of the following: trans random, cis random, trans meso ([m]), cis meso ([m]), trans racemo ([r]), or cis racemo ([r]) DABL diads. In various examples, at least a portion of or all of the polymeric and/or the oligomeric chain(s), independently, at least partially or completely, is/are atactic, isotactic, isoenriched, syndiotactic, syndioenriched, or any combination thereof. In various examples, at least a portion of or all of the polymeric and/or the oligomeric chain(s), independently, at least partially or completely, is/are cis atactic, cis isotactic, cis isoenriched, cis syndiotactic, cis syndioenriched, trans atactic, trans isotactic, trans isoenriched, trans syndiotactic, or trans syndioenriched, or any combination thereof. In various examples, at least a portion of or all of the polymeric and/or the oligomeric chain(s), at least partially or completely, comprise(s) crystalline and/or amorphous domains. In various examples, at least a portion of or all of the polymeric and/or the oligomeric chain(s), independently, comprise(s) one or more end group(s) independently at each occurrence chosen from hydrogen group (—H), hydroxyl group (—OH), carboxylic acid group (—CO₂H), chloride group (—Cl), azide group (—N₃), acyloxy group (—O₂CR, where R is a C₁ to C₂₀ alkyl group or a C₁ to C₂₀ aryl group), and alkoxyl group (—OR, where R is a C₁ to C₂₀ alkyl group or a C₁ to C₂₀ aryl group). In various examples, the PDABL composition comprises one or more homopolymer(s) comprising the DABL repeat unit(s), one or more copolymer(s) comprising the DABL repeat unit(s) and one or more non-DABL repeat unit(s), or any combination thereof. In various examples, the PDABL composition comprise(s) from about 1 mol % to about 50 mol % of the non-DABL repeat units. In various examples, the non-DABL repeat unit(s) is/are, independently at each occurrence, chosen from substituted and unsubstituted β-, γ-, δ-, and ω-lactone repeat units, ether group repeat units, carbonate group repeat units, amide group repeat units, and carbamate group repeat units. In various examples, the non-DABL repeat unit(s) comprise(s) the following structure:

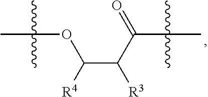

where R³ and R⁴ are, independently at each occurrence, a hydrogen group or a C₁, C₂, C₃, C₄, C₅, C₆, C₇, or a C₈ alkyl group, with the proviso that, for each non-DABL repeat unit, at least one of R³ and R⁴ is a hydrogen group. In various examples, the composition is in the form of a monolith, a film, a fiber, a flake, a pellet, a powder, a granule, a particle, a bead, a bar, a liquid, a solution, an emulsion, or any combination thereof. In various examples, the composition exhibits or has one or more or all of the following: a melting temperature (Tm) of about 100° C. to about 250° C.; an enthalpy of crystallization (ΔHc) of about 10 J/g to about 60 J/g; a decomposition temperature (Td) of about 240° C. to about 350° C.; a crystallization temperature (Tc) of about 10° C. to about 200° C.; a glass transition temperature (Tg) of about −20° C. to about 20° C.; an elongation at break of about 100% to about 1200%; or a tensile strength of about 5 MPa to about 50 MPa. In various examples, at least a portion of or all of the PDABL composition is at least partially or completely biodegradable.

In various examples, the method of forming the poly(α,β-dialkyl β-lactone) (PDABL) composition comprises forming a reaction mixture comprises: one or more α,β-dimethyl-β-lactone(s) (DAL(s)) (such as, for example, dimethyl-β-propiolactone); and one or more ring opening polymerization (ROP) initiator(s) and one or more ROP catalyst(s), one or more catalyst-initiator(s), one or more precursor(s) thereof, or any combination thereof, where the PDABL composition is formed. In various examples, the ROP catalyst-initiator(s) is/are chosen from organic salt(s), carbene(s), aromatic alcohol(s), metal alkoxide(s) and/or aryloxide(s), (multidentate ligand) metal alkoxide complex(es), (multidentate ligand) metal aryloxide complex(es), and any combination thereof, and where the metal is, independently at each occurrence, chosen from main group metals, transition metals and rare-earth metals. In various examples, the organic salt(s) is/are chosen from imidazolium salt(s), aminophosphonium salt(s), diphosphazenium salt(s), ammonium salt(s), and any combination thereof; and/or the alkoxide(s) and/or aryloxide(s) is/are chosen from C₁-C₂₀ alkoxide(s) and C₁-C₂₀ aryloxide(s). In various examples, the ROP catalyst-initiator(s) is/are chosen from: yttrium (III) tris(isopropoxide) (Y(OⁱPr)₃); magnesium (II) benzhydrol (Mg(BH)₂); zinc (II) benzhydrol (Zn(BH)₂); zinc (II) phenoxide isopropoxide ([L]Zn(OⁱPr) or a dimer thereof)₂, where L is a phenoxide ligand, independently at each occurrence, chosen from:

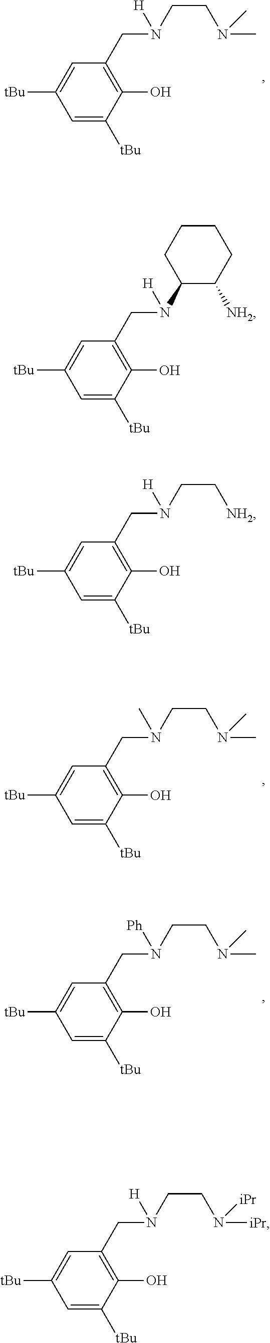 [SMILES: CN(C)CCNCc1cc(C(C)(C)C)cc(C(C)(C)C)c1O ; CC(C)(C)c1cc(CN[C@H]2CCCC[C@@H]2N)c(O)c(C(C)(C)C)c1 ; CC(C)(C)c1cc(CNCCN)c(O)c(C(C)(C)C)c1 ; CN(C)CCN(C)Cc1cc(C(C)(C)C)cc(C(C)(C)C)c1O ; CN(C)CCN(Cc1cc(C(C)(C)C)cc(C(C)(C)C)c1O)c1ccccc1 ; CC(C)N(CCNCc1cc(C(C)(C)C)cc(C(C)(C)C)c1O)C(C)C]

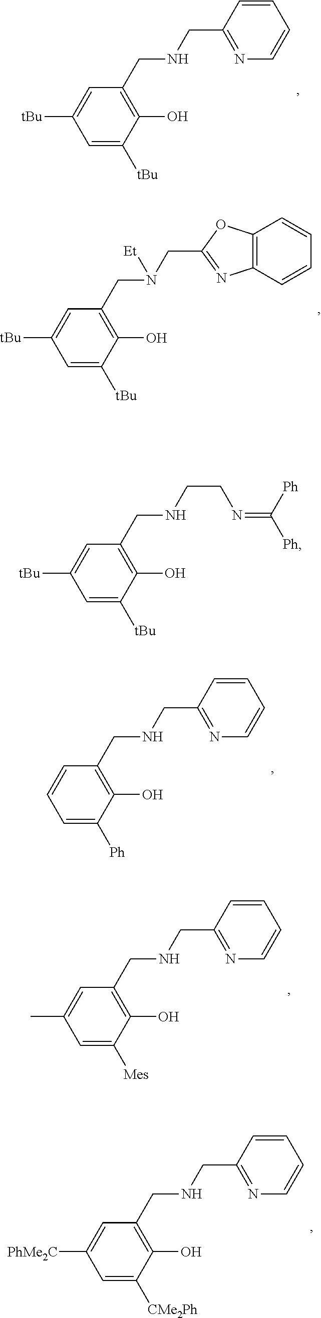

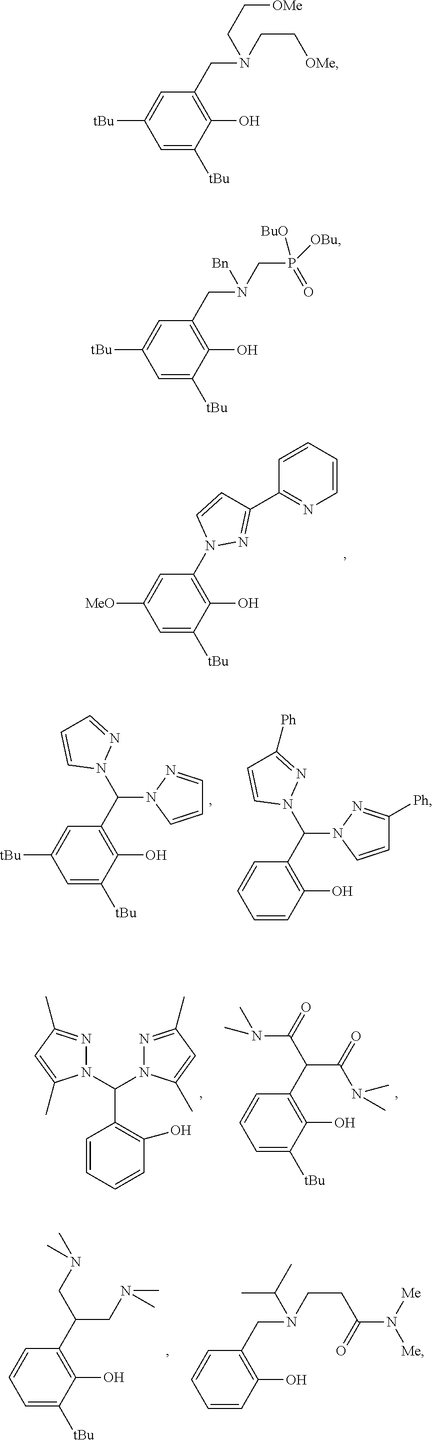

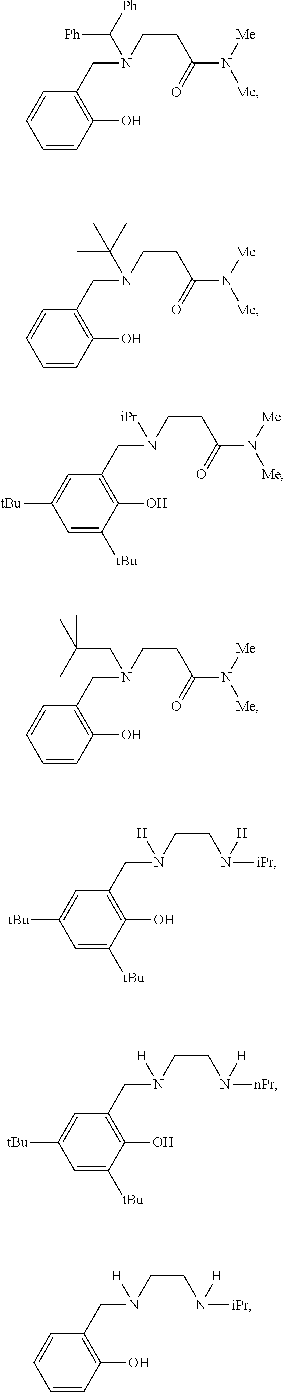

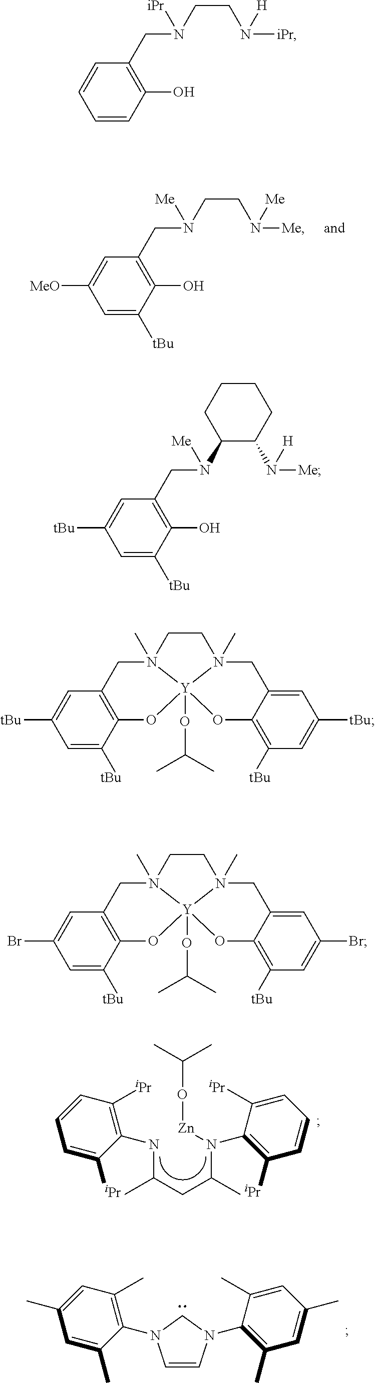

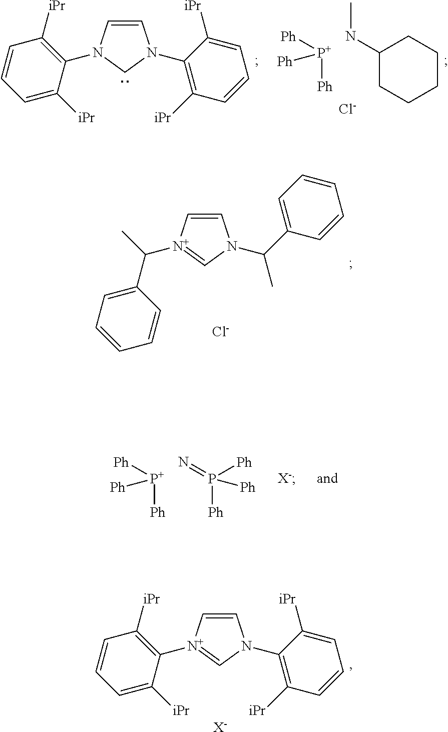

where X⁻ is, independently at each occurrence, chosen from RCO₂⁻, Cl⁻, HCO₃⁻, and N₃⁻, and where R is, independently at each occurrence, chosen from hydrogen group, aliphatic groups, and aryl groups; and any combination thereof. In various examples, one or more or all of the ROP initiator(s), the ROP catalyst(s), or the ROP catalyst-initiator(s) is/are formed by precursor(s) thereof in situ in the reaction mixture. In various examples, the reaction mixture comprises one or more ROP catalyst-initiators, precursor(s) thereof, or any combination thereof (e.g., the reaction mixture does not comprise a ROP initiator or a ROP catalyst, or a precursor thereof). In various examples, the reaction mixture further comprises one or more non-DAL monomer(s). In various examples, the non-DAL monomer(s) is/are, independently at each occurrence, chosen from substituted and unsubstituted β-, γ-, δ-, and ω-lactone(s), cyclic ether(s), cyclic carbonate(s), and cyclic carbamate(s). In various examples, the non-DAL monomer(s) is/are, independently at each occurrence, chosen from substituted and unsubstituted β-propiolactone, β-butyrolactone, β-valerolactone, β-caprolactone, lactides, and glycolides. In various examples, the reaction mixture comprises a molar ratio of the DAL(s) to the non-DAL monomer(s) of from about 50:50 to about 100:0. In various examples, the reaction mixture comprises from about 0.01 mol % to about 1 mol % of the ROP initiator(s) and/or the ROP catalyst(s), the ROP catalyst-initiator(s), precursor(s) thereof, or any combination thereof, based on the total moles of the DAL(s), the non-DAL monomer(s), and the ROP initiator(s) and the ROP catalyst(s), the ROP catalyst initiator(s), the precursor(s) thereof, or any combination thereof. In various examples, the reaction mixture further comprises one or more or organic solvent(s), or any combination thereof. In various examples, the organic solvent(s) is/are chosen from polar aprotic solvents, ether solvents, aromatic solvents, chlorinated solvents, lactone solvents, and any combination thereof. In various examples, the method further comprises forming an article of manufacture by molding, extrusion, blowing, casting, or spinning one or more of the PDABL composition(s).

In various examples, the article of manufacture comprises one or more of the PDABL composition(s). In various examples, the article of manufacture is in the form of a monolith, a coating, a sheet, a film, a fiber, a solid article, a hollow article, a foam, or a composite. In various examples, the article of manufacture is a packaging article, a single-use article, a sports article, a biomedical article, an agricultural article, an automotive article, or an electronic article. In various examples, the packaging article is a film, a wrapping, a sheet, a textile, a net, a bag, a container, a tub, a closure, a cap, a handle, a dispenser, a filler, a protector, a pad, or a fastener; the single-use article is a bag, a container, a dispenser, a cup, a bottle, a plate, cutlery, or a straw; the sports article is a fishing line; the biomedical article is a drug delivery article, a wound closure article, a wound dressing article, a surgical suture, a medical implant, or a tissue engineering construct; or the agricultural article is a film, a wrapping, a sheet, a textile, a net, a twine, a string, clips, wires, stakes, a bag, a container, a tub, a closure, a cap, a handle, a dispenser, a filler, a protector, a pad, a fastener, a bottle, a lid, a pot, or mulch. In various examples, the article of manufacture is biodegradable.

In various examples, the depolymerization method comprises: forming a depolymerization mixture comprising: one or more PDABL composition(s), where the PDABL composition(s) comprise(s) one or more homopolymer(s) comprising the DABL repeat unit(s); and one or more depolymerization catalyst(s); and heating the depolymerization mixture, thereby forming one or more depolymerization product(s). In various examples, the depolymerization catalyst(s) is/are chosen from non-metal oxide(s), group II metal oxide(s), group II metal aliphatic carboxylate(s), group II metal aromatic carboxylate(s), aliphatic organic acid(s), aromatic organic acid(s), aliphatic organic base(s), aromatic organic base(s), salts thereof, and any combination thereof. In various examples, the group II metal oxide(s) is/are chosen from magnesium oxide, magnesium tiglate, silica gel, sand, p-toluenesulfonic acid, 4-dimethylaminopyridine, and any combination thereof. In various examples, the depolymerization mixture comprises from about 1 weight percent (wt. %) to about 20 wt. % of the depolymerization catalyst(s), based on the total weight of the depolymerization mixture. In various examples, the depolymerization mixture is heated according to one or more or all of the following: at a temperature of from about 190° C. to about 220° C.; for a time of from about 1 hour (h) to about 12 h; or under inert conditions. In various examples, the depolymerization product is tiglic acid. In various examples, the method further comprising isolating and, optionally, purifying, the depolymerization product(s). In various examples, the depolymerization mixture comprises one or more article(s) of manufacture.

### BRIEF DESCRIPTION OF THE FIGURES

For a fuller understanding of the nature and objects of the disclosure, reference should be made to the following detailed description taken in conjunction with the accompanying figures herein.

FIG. 1 shows: (FIG. 1A) A preparation of poly(2-methyl-2-hydroxy-butyrate) (PMHB) from C4 feedstock; and (FIG. 1B) A polyester thermal decomposition pathway.

FIG. 2 shows: (FIG. 2A) A preparation of a racemic mixture (rac-1) of cis-2,3-dimethyl-β-propiolactone (1) by catalytic carbonylation of trans-2-buteneoxide (a racemic (rac) mixture) and a racemic mixture (rac-2) of trans-2,3-dimethyl-β-propiolactone (2) by catalytic carbonylation of cis-2-buteneoxide (a meso compound). (FIG. 2B) A preparation of an enantiomeric enriched (ee) mixture of 2 from cis-2-buteneoxide (a meso compound).

FIG. 3 shows (FIG. 3A) Stereochemistry of a lactone ring opening. (FIG. 3B) An atactic PMHB. (FIG. 3C) A syndiotactic PMHB. (FIG. 3D) An isotactic PMHB (cis and trans isotactic PMHB may also be obtained by isoselective polymerization of rac-1 and rac-2, respectively).

FIG. 4 shows a general reaction scheme and the metal alkoxide catalysts, organocatalysts, and organic cocatalysts used for the polymerizations of Table 1.

FIG. 5 shows a polymerization of rac-1 and rac-2 by zinc (β-diimine (BDI)) complex (BDI)Zn(OⁱPr).

FIG. 6 shows a polymerization of rac-1 and rac-2 by bis(triphenylphosphine)iminium adamantate complex [PPN][O₂CAd].

FIG. 7 shows a polymerization of rac-1 and rac-2 by yttrium bis(amino-methylphenoxide) complex (ONNO)Y(OⁱPr).

FIG. 8 shows various zinc phenoxide catalysts for polymerizations of Table 2.

FIG. 9 shows ¹³C NMR assignments: (FIG. 9A) trans PMHB (C═O peak) and (FIG. 9B) cis PMHB microstructures. (C—O peak).

FIG. 10 shows ¹³C NMR assignments of isoenriched cis and trans isotactic PMHB: superimposed plots.

FIG. 11 shows a powder x-ray diffraction (XRD) for cis and trans PMHB: (FIG. 11A) cis isotactic and cis atactic PMHB. (FIG. 11B) cis isotactic and trans isotactic PMHB.

FIG. 12 shows DSC traces for heating (second melt, 10° C./min) (FIG. 12A) and cooling (crystallization, −10° C./min) (FIG. 12B) transitions of cis PMHB microstructures.

FIG. 13 shows preparations of higher order copolymer architectures of PMHB.

FIG. 14 shows copolymerizations of mixtures of rac-1 and rac-2 under various conditions for Table 4.

FIG. 15 shows: (FIG. 15A) a preparation of a syndioenriched cis-co-trans PMHB using 80% cis (rac-1)/20% trans (rac-2) feed with a [ONN]Zn(OⁱPr) (with L1-1 ligand) catalyst. (FIG. 15B) Tensile testing of syndioenriched cis-co-trans PMHB.

FIG. 16 shows: (FIG. 16A) a preparation of syndiotactic cis-co-trans PMHB using 70% cis (rac-1)/30% trans (rac-2) and 50% cis (rac-1)/50% trans (rac-2) feeds with [ONNO]Y(OⁱPr) catalyst. (FIG. 16B) Tensile testing of syndiotactic cis-co-trans PMHB.

FIG. 17 shows a general design of a new polyhydroxyalkanoate, PHMB. (FIG. 17A) Structural evolution from iPP, R-P3HB to PHMB. (FIG. 17B) Comparison in properties of PHMB with various commercial polymers, such as (bio)degradable polyhydroxylbutyrate (PHB), polylactic acid (PLA), polycaprolactone (PCL), poly(butylene adipate terephthalate) (PBAT), and nondegradable iPP and high-density polyethylene (HDPE). (FIG. 17C) Streamlined synthesis of a PHMB from 2-butene via epoxidation, carbonylation and polymerization.

FIG. 18 shows a comparison of selected regions in (FIG. 18A) ¹H NMR spectra and (FIG. 18B) quantitative ¹³C NMR spectra of cis-PHMBs bearing different tacticity.

FIG. 19 shows (FIG. 19A) selected regions in a quantitative ¹³C NMR spectrum of cis-PHMB, Pᵣ=0.75, and comparison of selected region in (FIG. 19B) ¹H NMR spectra and (FIG. 19C) quantitative ¹³C NMR spectra of PHMB copolymers bearing different cis content.

FIG. 20 shows an OTREP structure of B-HMDS (recrystallized from toluene).

FIG. 21 shows a PXRD overlay of cis-PHMB homopolymers with different tacticity, from completely atactic (Pᵣ=0.50) to highly syndiotactic (Pᵣ=0.95).

FIG. 22 shows a polymerization of cis-DMPL by different initiators, and thermal and mechanical properties of the resulting homopolymers (Tₘ refers to the major melting peak observed in DSC).

FIG. 23 shows a synthesis and characterization of PHMB copolymers with different cis content. (FIG. 23A) Polymerization of cis/trans mixture of DMPL with varied cis content. (FIG. 23B) Thermal properties of the resulting copolymers. (FIG. 23C) Tensile curves of different PHMB copolymers with 90%, 80% and 70% cis blends and their comparison with commercial polyolefin plastics, with photos showing specimens before and after being stretched. Tₘ refers to the major melting peak observed in DSC and T_{c} refers to the crystallization peak.

FIG. 24 shows a Mₙ change of 90% cis PHMB copolymer over time at 170° C.

FIG. 25 shows time-conversion plots in the copolymerization of cis- and trans-DMPL mixtures.

FIG. 26 shows potential chemical recycling and upcycling of PHMB: depolymerization to tiglic acid.

FIG. 27 shows an experimental setup for the depolymerization of mixtures of various PHMB samples.

FIG. 28 shows copolymerizations of cis- and trans-DMPL with [ONN]Zn(OⁱPr) (with various ligands) catalyst.

FIG. 29 shows homopolymerization of trans-DMPL with [ONN]Zn(OⁱPr) (with various ligands) catalyst.

FIG. 30 shows tensile data of copolymers of cis- and trans-DMPL with [ONN]Zn(OⁱPr) (with L1-4 or L6-2 ligand) catalyst.

FIG. 31 shows homopolymerization of cis-DMPL with expanded ligand library.

FIG. 32 shows polymerization of β-lactones using various catalysts.

### DETAILED DESCRIPTION OF THE DISCLOSURE

Although claimed subject matter will be described in terms of certain examples, other examples, including examples that do not provide all of the benefits and features set forth herein, are also within the scope of this disclosure. Various structural, logical, and process step changes may be made without departing from the scope of the disclosure.

As used herein, unless otherwise indicated, “about”, “substantially”, or “the like”, when used in connection with a measurable variable (such as, for example, a parameter, an amount, a temporal duration, or the like) or a list of alternatives, is meant to encompass variations of and from the specified value including, but not limited to, those within experimental error (which can be determined by, e.g., a given data set, an art accepted standard, etc. and/or with, e.g., a given confidence interval (e.g. 90%, 95%, or more confidence interval from the mean), such as, for example, variations of +/−10% or less, +/−5% or less, +/−1% or less, and +/−0.1% or less of and from the specified value), insofar such variations in a variable and/or variations in the alternatives are appropriate to perform in the instant disclosure. As used herein, the term “about” may mean that the amount or value in question is the exact value or a value that provides equivalent results or effects as recited in the claims or taught herein. That is, it is understood that amounts, sizes, compositions, parameters, and other quantities and characteristics are not and need not be exact, but may be approximate and/or larger or smaller, as desired, reflecting tolerances, conversion factors, rounding off, measurement error, or the like, or other factors known to those of skill in the art such that equivalent results or effects are obtained. In general, an amount, size, composition, parameter, or other quantity or characteristic, or alternative is “about” or “the like,” whether or not expressly stated to be such. It is understood that where “about,” is used before a quantitative value, the parameter also includes the specific quantitative value itself, unless specifically stated otherwise.

Ranges of values are disclosed herein. The ranges set out a lower limit value and an upper limit value. Unless otherwise stated, the ranges include the lower limit value, the upper limit value, and all values between the lower limit value and the upper limit value, including, but not limited to, all values to the magnitude of the smallest value (either the lower limit value or the upper limit value) of a range. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “0.1% to 5%” should be interpreted to include not only the explicitly recited values of 0.1% to 5%, but also, unless otherwise stated, include individual values (e.g., 1%, 2%, 3%, and 4%) and the sub-ranges (e.g., 0.5% to 1.1%; 0.5% to 2.4%; 0.5% to 3.2%, and 0.5% to 4.4%, and other possible sub-ranges) within the indicated range. It is also understood (as presented above) that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about, it will be understood that the particular value forms a further disclosure. For example, if the value “about 10” is disclosed, then “10” is also disclosed.

As used herein, unless otherwise stated, the term “group” refers to a chemical entity that is monovalent (i.e., has one terminus that can be covalently bonded to other chemical species), divalent, or polyvalent (i.e., has two or more termini that can be covalently bonded to other chemical species). The term “group” also includes, but is not limited to, radicals (e.g., monovalent and multivalent, such as, for example, divalent radicals, trivalent radicals, and the like). Illustrative, non-limiting examples of groups include:

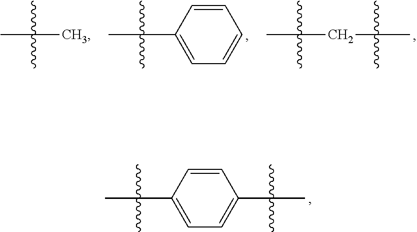

and the like.

As used herein, unless otherwise indicated, the term “aliphatic group” refers to branched or unbranched hydrocarbon groups that, optionally, contain one or more degree(s) of unsaturation. Degrees of unsaturation include, but are not limited to, carbon-carbon double bonds and carbon-carbon triple bonds. Non-limiting examples, of aliphatic groups with one or more degree(s) of unsaturation include alkenyl groups, alkynyl groups, and aliphatic cyclic groups, and the like An aliphatic group may be an alkyl group. In various examples, an aliphatic group is a C₁ to C₂₀ aliphatic group, including all integer numbers of carbons and ranges of numbers of carbons therebetween (e.g., C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, and C₂₀). An aliphatic group maybe unsubstituted or substituted with one or more substituent(s). Examples of substituents include, but are not limited to, various substituents such as, for example, halide groups (—F, —Cl, —Br, and —I), aliphatic groups (e.g., alkyl groups, alkenyl groups, alkynyl groups, and the like), halogenated aliphatic groups (e.g., trifluoromethyl group and the like), aryl groups, halogenated aryl groups, hydroxyl groups, amine groups, nitro groups, cyano groups, isocyano groups, silyl groups, alkoxide groups, alcohol groups, ether groups, ketone groups, carboxylate groups, carboxylic acid groups, ester groups, amide groups, thioether groups, and the like, and any combination thereof.

As used herein, unless otherwise indicated, the term “alkyl group” refers to branched or unbranched hydrocarbon groups that include only single bonds between carbon atoms. In various examples, an alkyl group is a C₁ to C₂₀ alkyl group, including all integer numbers of carbons and ranges of numbers of carbons therebetween (e.g., a C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, and C₂₀). In various examples, an alkyl group is a saturated group. In various examples, an alkyl group is a cyclic alkyl group, e.g., a monocyclic alkyl group or a polycyclic alkyl group. Examples of alkyl groups include, but are not limited to, methyl groups, ethyl groups, propyl groups, butyl groups, isopropyl groups, tert-butyl groups, benzyl groups, cyclohexyl groups, adamantyl groups, and the like. In various examples, an alkyl group is unsubstituted or substituted with one or more substituent(s). Examples of substituents include, but are not limited to, various substituents such as, for example, halide groups (—F, —Cl, —Br, and —I), aliphatic groups (e.g., alkyl groups, alkenyl groups, alkynyl groups, and the like), halogenated aliphatic groups (e.g., trifluoromethyl group and the like), aryl groups, halogenated aryl groups, hydroxyl groups, amine groups, nitro groups, cyano groups, isocyano groups, silyl groups, alkoxide groups, alcohol groups, ether groups, ketone groups, carboxylate groups, carboxylic acid groups, ester groups, amide groups, thioether groups, and the like, and any combination thereof.

As used herein, unless otherwise indicated, the term “aryl group” refers to C₅ to C₃₀ aromatic or partially aromatic carbocyclic groups, including all integer numbers of carbons and ranges of numbers of carbons therebetween (e.g., C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, and C₃₀). Aryl groups may comprise polyaryl groups such as, for example, fused ring groups, biaryl groups, or the like, or any combination thereof. The aryl group may be unsubstituted or substituted with one or more substituent(s). Examples of substituents include, but are not limited to, substituents such as, for example, halide groups (—F, —Cl, —Br, and —I), aliphatic groups (e.g., alkyl groups, alkenyl groups, alkynyl groups, and the like), halogenated aliphatic groups (e.g., trifluoromethyl group and the like), aryl groups, halogenated aryl groups, hydroxyl groups, amine groups, nitro groups, cyano groups, isocyano groups, silyl groups, alkoxide groups, alcohol groups, ether groups, ketone groups, carboxylate groups, carboxylic acid groups, ester groups, amide groups, thioether groups, and the like, and any combination thereof. Aryl groups may contain hetero atoms, such as, for example, oxygen, nitrogen (e.g., pyridinyl groups and the like), sulfur, and the like, and any combination thereof. Examples of aryl groups include, but are not limited to, phenyl groups, biaryl groups (e.g., biphenyl groups and the like), fused ring groups (e.g., naphthyl groups and the like), hydroxybenzyl groups, tolyl groups, xylyl groups, furanyl groups, benzofuranyl groups, indolyl groups, imidazolyl groups, benzimidazolyl groups, pyridinyl groups, and the like.

As used herein, unless otherwise indicated, the term “hydrocarbyl group” includes, but is not limited to, the terms “aliphatic group” and “aryl group”.

As used herein, unless otherwise indicated, the term “structural analog” refers to a compound or group that can be envisioned to arise from another compound or group, respectively, if one atom or group of atoms, functional groups, or substructures is replaced with another atom or group of atoms, functional groups, substructures, or the like.

As used herein, unless otherwise indicated, the term “precursor” refers to a compound that can be converted into a ROP initiator, a ROP catalyst, or a ROP catalyst-initiator by chemical reaction.

The present disclosure provides poly(dialkyl β-lactone) (PDABL) compositions and compositions comprising one or more polymeric chain(s), one or more oligomeric chain(s), or the like, or any combination thereof, the polymeric chain(s) and/or the oligomeric chain(s) each comprising a plurality of dialkyl β-lactone (DABL) repeat unit(s). The present disclosure also describes methods of making the compositions, methods using the compositions, methods of depolymerizing the compositions, and uses of the compositions.

As used herein, unless otherwise indicated, a PDABL composition comprises one or more DABL homopolymer(s), while a composition comprising one or more polymeric chain(s), one or more oligomeric chain(s), or the like, or any combination thereof, the polymeric chain(s) and/or the oligomeric chain(s) each comprising a plurality of dialkyl β-lactone (DABL) repeat unit(s), comprises one or more PDABL homopolymer(s) and/or one or more PDABL copolymer(s). Compositions comprising the PDABL homopolymer(s) and/or the PDABL copolymer(s) are collectively referred to herein, unless otherwise indicated, as “PDABL compositions”.

In an aspect, the present disclosure provides PDABL compositions. In various examples, a PDABL composition is made by a method of the present disclosure. Non-limiting examples of PDABL compositions are described herein.

In various examples, a PDABL composition is a (poly(2-alkyl-3-hydroxyalkanoate) (PAHA) composition (also referred to herein as a 2,3-PDABL composition or α,β-PDABL composition) and the DABL repeat units are 2-alkyl-3-hydroxyalkanoate (AHA) repeat units (also referred to herein as 2,3-DABL repeat units or α,β-DABL repeat units). In various examples, a PAHA composition can be made by polymerization of a 2,3-dialkyl-β-lactone (2,3-DAL) (also referred to herein as an α,β-dialkyl-β-lactone (α,β-DAL)).

In various examples, at least a portion of or all of the 2-alkyl groups of a PAHA composition is/are, independently, chosen from C₁, C₂, C₃, C₄, C₅, C₆, C₇, and C₈ alkyl group(s). In various examples, at least a portion of or all of 3-hydroxyalkanoate groups of a PAHA composition comprise(s) an alkanoate group which is/are, independently, Cₙ₊₂, where n is the number of carbons of the 2-alkyl group.

In various examples, a PDABL composition comprises (e.g., the one or more polymeric chain(s), one or more oligomeric chain(s), or the like, or any combination thereof, of the PDABL composition comprises) a plurality of DABL repeat unit(s). In various examples, at least a portion of or all of the DABL repeat units are contiguous. In various examples, one or more or all DABL repeat unit(s) of a PDABL composition, independently, have the following structure:

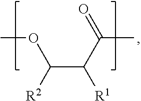

where R¹ and R² are each, independently, chosen from C₁, C₂, C₃, C₄, C₅, C₆, C₇, and C₈ alkyl groups. In various examples, where R¹ and R² are each, independently, chosen from C₁, C₂, C₃, and C₄, alkyl groups. In various examples, R¹ and R² are independently at each occurrence chosen from methyl group, ethyl group, n-propyl group, isopropyl groups, butyl groups, pentyl groups. hexyl groups, heptyl groups, octyl groups, and the like. In various examples, at least a portion of or all alkyl groups of DABL repeat units (e.g., AHA repeat units or the like) of a PDABL composition (e.g., PAHA composition or the like) are methyl groups. In various examples, R¹ and R² of a DABL repeat unit are the same or different. In various examples, R¹ and R² of a DABL repeat unit are the same (e.g., a 2-methyl-3-hydroxybutyrate (MHB) repeat unit). In various examples, R¹ and R² of a DABL repeat unit are different.

In various examples, a PDABL composition comprises one or more same DABL repeat unit(s). In various examples, a PDABL composition is a poly(2-methyl-3-hydroxybutyrate) (PMHB) composition and the DABL repeat units are 2-methyl-3-hydroxybutyrate (MHB) repeat units. In various examples, a PDABL composition comprises one or more different DABL repeat unit(s).

In various examples, a PDABL composition comprises one of more or all of the following: a molecular weight (Mw and/or Mn) as described herein (such as, for example, a (Mw and/or Mn) of about 50 kiloDalton (kD) or greater, about 60 kD or greater, about 80 kD or greater, about 100 kD or greater, or about 150 kD or greater or the like); one or more crystalline domain(s); a combination of cis and trans DABL repeat units; at least partially or all atactic DABL repeat units, at least partially or all syndiotactic DABL repeat units, or any combination thereof; one or more non-DABL repeat unit(s); or two or more DABL repeat units with different R¹ groups, R² groups, or both.

A PDABL composition can comprise various PDABL homopolymer(s), PDABL copolymer(s), and the like, and any combination thereof. In various examples a PDABL homopolymer comprises 100 mol % of the same cis or the same trans DABL repeat units (e.g., 100 mol % of the same cis or the same trans AHA repeat units or the like). In various examples, a PDABL copolymer comprises one or more different DABL repeat unit(s) from any other DABL repeat units. In various examples, a different DABL repeat unit has a different R¹ and/or R² group from any other DABL repeat units. In various examples, a different DABL repeat unit is a different geometric isomer (e.g., cis vs. trans) from any other DABL repeat units. In various examples, a different DABL repeat unit is a different enantiomer (e.g., (R) vs. (S)) from any other DABL repeat units. In various examples, a PDABL copolymer comprises one or more same and/or different DABL repeat unit(s) and one or more non-DABL repeat unit(s). In various examples. In various examples, a PDABL copolymer comprises one or more of the same cis and/or the same trans DABL repeat unit(s) and one or more non-DABL repeat unit(s). In various examples, a PDABL composition comprises (or is) one or more PDABL homopolymer(s), one or more PDABL copolymer(s), or the like, or any combination thereof. In various examples, a PDABL copolymer comprises (or is) a random copolymer, an alternating copolymer, a tapered copolymer, a graft copolymer, a block copolymer, a dendritic copolymer, or the like, or any combination thereof.

In various examples, a PDABL composition comprises one or more DABL repeat unit(s) and/or one or more DABL repeat unit diad(s) that is/are different from the other DABL repeat units and/or DABL repeat unit diad(s). In various examples, a PDABL composition comprises two or more (e.g., three or more, four or more, and the like) different DABL repeat units and/or DABL repeat unit diads. In various examples, a different DABL repeat unit comprises different R¹ and/or R² groups, is a different enantiomer (e.g., (R) vs. (S)), is a different a geometric isomer (e.g., cis v. trans), or the like, or any combination thereof, from any other DABL repeat units. In various examples, a different DABL repeat unit diad comprises a tacticity (e.g., meso (m) vs. raceme (r)), or the like, or any combination thereof, that is different from the other DABL repeat unit diads. In various examples, a PDABL copolymer further comprises a plurality of non-DABL repeat unit(s).

A PDABL composition can comprise various geometrical isomers of the DABL repeat units. The presence of cis and/or trans DABL repeat units can be determined by methods known in the art. In various examples, the presence of cis and/or trans DABL repeat units is determined (e.g., measured) by ¹H nuclear magnetic resonance (NMR), ¹³C NMR (e.g., quantitative ¹³C NMR by peak integration), or the like, or any combination thereof. In various examples, at least a portion of or all of the polymeric chain(s) and/or the oligomeric chain(s) comprise(s), independently, at least partially or completely, cis and/or trans DABL (e.g., cis and/or trans AHA, such as, for example, cis and/or trans MHB and the like) repeat units. In various examples, at least a portion of or all of the polymeric chain(s) and/or the oligomeric chain(s) comprise(s), independently, 100 mol % cis or 100 mol % trans DABL repeat units, based on the total moles of DABL repeat units. In various examples, at least a portion of or all of the polymeric chain(s) and/or the oligomeric chain(s), comprise(s), independently, DABL repeat units having a cis to trans molar DABL repeat unit ratio of about 1: about 99 to about 99: about 1, including all 0.1 cis to trans molar DABL repeat unit ratio values and ranges therebetween (e.g., about 95:5 to about 5:95, about 90:10 to about 10:90, about 80:20 to about 20:80, about 70:30 to about 30:70, about 60:40 to about 40:60, or about 50:50 to about 99:1, about 70:30 to about 99:1, about 80 20 to about 99 1, or about 90:1 to about 99:1, or about 50:50 to about 1:99, about 70:30 to about 1:99, about 80:20 to about 1:99, about 10:90 to about 1:99, or about 5:95 to about 1:99). In various examples, at least a portion of or all of the polymeric chain(s) and/or the oligomeric chain(s), comprise(s) a majority of cis DABL repeat units.

A PDABL composition can comprise various stereoregularities of the DABL diads. The presence of stereoregular DABL diads (e.g., meso (m) and/or racemo (r) DABL diads) can be determined by methods known in the art. In various examples, the presence of meso (m) and/or racemo (r) DABL diads (e.g., meso (m) and/or racemo (r) AHA diads, such as, for example, meso (m) and/or racemo (r) MHB diads and the like) is determined (e.g., measured) by ¹H nuclear magnetic resonance (NMR), ¹³C NMR (e.g., quantitative ¹³C NMR by peak integration), or the like, or any combination thereof. In various examples, at least a portion of or all of the polymeric chain(s) and/or the oligomeric chain(s) comprise(s), independently, at least partially or completely, randomly oriented and/or stereoregular DABL diads (e.g., randomly oriented and/or stereoregular AHA diads, such as, for example, randomly oriented and/or stereoregular MHB diads and the like). In various examples, at least a portion of or all of the polymeric chain(s) and/or the oligomeric chain(s) comprise(s), independently, meso (m) DABL diads and racemo (r) DABL diads (e.g., meso (m) AHA diads, such as, for example, meso (m) MHB diads and the like, and/or racemo (r) AHA diads, such as, for example, racemo (r) MHB diads and the like).

In various examples, at least a portion of or all of the polymeric chain(s) and/or the oligomeric chain(s) comprise(s), independently, at least partially or completely, trans random, cis random, trans meso ([m]), cis meso ([m]), trans racemo ([r]), or cis racemo ([r]) DABL diads, or the like, or any combination thereof. In various examples, at least a portion of or all of the polymeric chain(s) and/or the oligomeric chain(s), independently, at least partially or completely, comprise(s) about 50 mol % to about 100 mol %, including all 0.1 mol % values and ranges therebetween, of any one of the following: trans random, cis random, trans meso ([m]), cis meso ([m]), trans racemo ([r]), or cis racemo ([r]) DABL diads. In various examples, at least a portion of or all of the polymeric chain(s) and/or the oligomeric chain(s) comprise(s), independently, about 50 mol % to about 100 mol % of cis DABL diads including all 0.1 mol % values and ranges therebetween.

In various examples, the polymeric chain(s) and/or the oligomeric chain(s), independently, do not comprise(s) completely trans isotactic DABL diads. In various examples, the polymeric chain(s) and/or the oligomeric chain(s) comprise(s), independently, about 99 9 mol % or less of trans isotactic DABL diads (e.g., about 99 mol % or less, about 95 mol % or less, about 90 mol % or less, about 80 mol % or less, about 70 mol % or less, about 60 mol % or less, or about 50 mol % or less). In various examples, the polymeric chain(s) and/or the oligomeric chain(s) comprise(s), independently, from about 50 mol % to about 99.9 mol % of trans isotactic DABL diads, including all 0.1 mol % values and ranges therebetween (e.g., from about 50 mol % to about 99 mol %, from about 50 mol % to about 95 mol %, from about 50 mol % to about 90 mol %, or from about 50 mol % to about 80 mol %).

A PDABL composition can have various types of tacticity. In various examples, at least a portion of or all of the polymeric chain(s) and/or the oligomeric chain(s) is/are, independently, at least partially or completely, atactic, isotactic, isotactic enriched, syndiotactic, syndioenriched, or the like, or any combination thereof. In various examples, at least a portion of or all of the polymeric chain(s) and/or the oligomeric chain(s) is/are, independently, trans atactic, cis atactic, trans isotactic, cis isotactic, trans isoenriched, cis isoenriched, trans syndiotactic, cis syndiotactic, cis syndioenriched, trans syndioenriched, or the like, or any combination thereof. In various examples, at least a portion of or all of the polymeric chain(s) and/or the oligomeric chain(s) is/are, independently: about 70% cis/about 30% trans (molar ratio) with cis-syndiotactic/trans-syndioenriched; about 80% cis/about 20% trans (molar ratio) with cis-syndioenriched/trans-syndioenriched; about 60% cis/about 40% trans (molar ratio) with cis-syndiotactic/trans-syndioenriched; or about 50% cis/about 50% trans (molar ratio) with cis-syndiotactic/trans-syndioenriched.

In various examples, at least a portion of or all of the polymeric chain(s) and/or the oligomeric chain(s) comprise(s), independently, less than 100 mol % of the DABL repeat units (e.g., a PDABL copolymer composition). In various examples, at least a portion of or all of the polymeric chain(s) and/or oligomeric chain(s) comprise(s), individually, about 1 mol % to about 50 mol % of non-DABL repeat units (e.g., non-AHA repeat units, such as, for example, non-MHB repeat units and the like), including all 0.1 non-DABL repeat unit mol % values and ranges therebetween (e.g., about 1 mol % to about 30 mol % of non-DABL repeat units).

Non-limiting examples of non-DABL repeat units include non-DABL ester repeat units (e.g., non-AHA β-, γ-, δ-, and ω-lactone repeat units), ether group repeat units (e.g., polyether repeat units or the like), carbonate group repeat units (e.g., polycarbonate repeat units or the like), acetal group repeat units (e.g., polyacetal repeat units or the like), amide group repeat units (e.g., polyamide repeat units or the like), carbamate group repeat units, alkyl group repeat units (e.g., polyolefin repeat units or the like), structural analogs thereof, and the like, and any combination thereof. In various examples, the non-DABL ester repeat units is/are non-AHA β-hydroxyalkanoate repeat units (e.g., β-hydroxybutyrate repeat units, β-hydroxyvalerate repeat units, and the like). In various examples, the non-DABL repeat unit(s) comprise(s) the following structure:

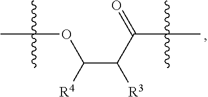

where R³ and R⁴ are each independently at each occurrence a hydrogen group or an alkyl group comprising 1 to 10 carbon atoms, with the proviso that at least one of R³ and R⁴ independently is a hydrogen. In various examples, at least a portion of or all of the polymeric chain(s) and/or the oligomeric chain(s) comprise(s), individually, about 50 mol % of the non-DABL repeat units comprising the following structure:

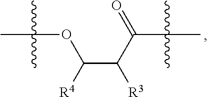

where R³ and R⁴ are each independently at each occurrence a hydrogen group or an alkyl group comprising 1 to 10 carbon atoms, with the proviso that at least one of R³ and R⁴ independently is a hydrogen.

Additional non-limiting examples of non-DABL repeat units, when the PDABL is a PMHB, include non-MHB DABL repeat units, where one alkyl group (the R¹ group or the R² group) of each non-MHB DABL repeat unit is, independently at each occurrence, a C₂, C₃, C₄, C₅, C₆, C₇, or C₈ group, with the proviso that the remaining alkyl group of each non-MHB DABL repeat unit is a methyl group, or where both alkyl groups of each non-MHB DABL repeat unit is/are each, independently, a C₂, C₃, C₄, C₅, C₆, C₇, or C₈ group.

In various examples, the DABL and/or the non-DABL repeat units are arranged by composition or by tacticity such that regions (e.g., blocks or the like) of specific composition and/or tacticity are formed within the copolymer. In various examples, the regions (e.g., blocks or the like) of composition and/or tacticity are arranged randomly, in alternating fashion, as a graft or as a dendrimer, or the like, or any combination thereof. In various examples, a polymeric or oligomeric chain can at least partially or completely comprise one or more copolymer (e.g., a random copolymer, alternating copolymer, tapered copolymer, block copolymer, graft copolymer, or dendritic copolymer, or the like, or any combination thereof) domain(s), each polymeric or oligomeric chain independently comprising DABL repeat units and non-DABL repeat units (e.g., MHB repeat units and the like and non-MHB repeat units and the like).

In various examples, the non-DABL, repeat units (e.g., non-MHB repeat units and the like) are formed from monomers that are polymerized by ring-opening, polymerized by other polymerization reactions (such as, for example, addition polymerizations, condensation polymerizations, or the like, or any combination thereof) or may be incorporated into initiators. In various examples, the PDABL composition is produced by the polymerization (e.g., ring opening polymerization or the like) of one or more 2,3-dialkyl-β-propiolactone monomer(s) and optionally with one or more monosubstituted alkyl-β-propiolactone monomer(s).

A PDABL composition can have various molecular weights (M_{w} and/or Mₙ). The molecular weight (M_{w} and/or Mₙ) value(s) and/or polydispersity index (PDI=Ð=M_{w}/Mₙ) of a PDABL composition can be determined by methods known in the art. In various examples, the molecular weight (M_{w} and/or Mₙ) value(s) and/or polydispersity index (PDI=Ð=M_{w}/Mₙ) of a PDABL composition is determined by gel permeation chromatography (GPC), or the like. In various examples, the one or more oligomeric chain(s), or the like, or any combination thereof, has/have: a weight average molecular weight (M_{w}) and/or a number average molecular weight (Mₙ), of about 1 kiloDalton (kD) to about 500 kD, including all 0.1 kD values and ranges therebetween (e.g., about 1 kD to about 400 kD, about 1 kD to about 300 kD, about 25 kD to about 400 kD, or about 75 kD to about 300 kD); and/or a polydispersity index (PDI=Ð=M_{w}/Mₙ) of about 1 to about 10, including all 0.1 PDI values and ranges therebetween (e.g., about 1 to about 1.5, about 1 to about 2, about 1 to about 3 about, or about 1 to about 5).

A PDABL composition can comprise various end groups. In various examples, the one or more oligomeric chain(s), or the like, or any combination thereof, comprise(s), independently, end groups chosen from hydrogen group (—H), hydroxyl group (—OH), carboxylic acid group (—CO₂H), chloride group (—Cl), azide group (—N₃), acyloxyl group (—O₂CR where R is a C₁ to C₂₀ alkyl group or a C₁ to C₂₀ aryl group), alkoxyl group (—OR, where R is a C₁ to C₂₀ alkyl group, e.g., a benzyl group, or a C₁ to C₂₀ aryl group), structural analogs thereof, and the like, and any combination thereof. In various examples, acyloxy group(s) is/are chosen from benzoyl groups, succinyl groups, adipyl groups, adamantylcarboxyl groups, and the like, and any combination thereof.

A PDABL composition can comprise various crystallinities. The presence of crystalline domains can be determined by methods known in the art. In various examples, the presence of crystalline domains in a PDABL composition is determined (e.g., measured) by powder x-ray diffraction (PXRD), dynamic scanning calorimetry (DSC), or the like, or any combination thereof. In various examples, the polymeric chains and/or the oligomeric chains comprise(s), individually, at least partially or completely, one or more crystalline and/or one or more amorphous domains. In various examples, a PDABL composition is semicrystalline. In various examples, a PDABL composition is semicrystalline and a solid at room temperature.

A PDABL composition can have various properties. Properties can be measured by methods known in the art. Thermal properties include, but are not limited to, melting temperature (Tm), enthalpy of crystallization (ΔHc), crystallization temperature (Tc), decomposition temperature (Td), glass transition temperature (Tg), and the like. In various examples, one or more or all of the thermal properties of a PDABL composition are determined (e.g., measured) by dynamic scanning calorimetry (DSC), thermogravometric analysis (TGA), or the like, or any combination thereof. Tensile properties include, but are not limited to, elongation at break, tensile strength, and the like. In various examples, the elongation at break and/or tensile strength of a PDABL composition are determined (e.g., measured) by uniaxial tensile testing (e.g., as described herein) or the like, or any combination thereof. In various examples, a PDABL composition exhibits one or more thermal and/or tensile propert(ies) substantially the same as or better than those exhibited by a comparable polyethylene or polypropylene.

In various examples a PDABL composition exhibits or has one or more or all of the following: a melting temperature (Tm), e.g., as measured by differential scanning calorimetry (DSC), of about 120° C. to about 250° C. (e.g., about 100° C. to about 250° C.), including all 0.1° C. values and ranges therebetween; an enthalpy of crystallization (ΔHc), e.g., as measured by DSC, of about 10 J/g to about 60 J/g, including all 0.1 J/g values and ranges therebetween; a decomposition temperature (Td), e.g., as measured by thermogravometric analysis (TGA), of about 240° C. to about 350° C., including all 0.1° C. values and ranges therebetween; a crystallization temperature (Tc), e.g., as measured by DSC, of about 10° C. to about 200° C. (e.g., about 10° C. to about 70° C., about 10° C. to about 135° C., about 50° C. to about 200° C., or about 130° C. to about 170° C.), including all 0.1° C. values and ranges therebetween; a glass transition temperature (Tg), e.g., as measured by DSC, of about −20° C. to about 20° C. (e.g., about 0° C. to about 20° C., or about 10° C. to about 20° C.), including all 0.1° C. values and ranges therebetween; an elongation at break, e.g., as measured by tensile testing, of from about 100% to about 1200% (e.g., about 200% to about 1200%, or about 300% to about 1200%) including all integer % values and ranges therebetween; or a tensile strength, e.g., as measured by tensile testing, of from about 5 MPa to about 50 MPa, including all 0.1 MPa values and ranges therebetween.

In various examples, a PDABL composition has a crystallization temperature (Tc) from about 130° C. to about 170° C., including all 0.1° C. values and ranges therebetween, and is cis atactic, cis syndiotactic, and/or cis isotactic. In various examples, a PDABL composition has a crystallization temperature (Tc), e.g., as measured by DSC, from about 10° C. to about 135° C., including all 0.1° C. values and ranges therebetween, and are trans-syndiotactic and/or trans-isotactic. In various examples, a PDABL composition has a glass transition temperature (Tg), e.g., as measured by DSC, of about −20° C. to about 20° C. (e.g., about 0° C. to about 20° C., or about 10° C. to about 20° C.), including all 0.1° C. values and ranges therebetween, and are trans atactic, trans syndiotactic and/or trans isotactic, cis atactic, or cis syndiotactic and/or cis isotactic.

A PDABL composition can exhibit various degradation properties. In various examples, the polymeric chain(s) and/or oligomeric chain(s), independently, is/are at least partially or completely degradable (or biodegradable). In various examples, the polymeric chain(s) and/or oligomeric chain(s) is/are, individually, at least partially or completely degradable (e.g., biodegradable) in bodily tissue, fluid, or organs; soil, compost, or other organic matter; fresh water, salt-water, or the like. In various examples, the polymeric chain(s) and/or the oligomeric chain(s) is/are, individually, least partially or completely degradable (e.g., biodegradable) by microbial degradation, hydrolysis, photodegradation, or the like, or any combination thereof. In various examples, the one or more polymeric and/or oligomeric chain(s) is/are, individually, at least partially or completely degradable (or biodegradable) under aerobic conditions, anaerobic conditions, or the like, or any combination thereof.

A PDABL composition can further comprise one or more additional polymer(s). Non-limiting examples of additional polymers include polyesters, structural analogs thereof, and the like. In various examples, a PDABL composition is a blend of one or more PDABL composition(s) and one or more polyester(s) and/or structural analogs thereof.

A PDABL composition can have various forms. In various examples, a PDABL composition is in the form of a monolith, a film, a fiber, a flake, a pellet, a powder, a granule, a particle, a bead, a bar, a liquid, a solution, an emulsion, or the like, or any combination thereof.

A PDABL composition can be used in a chemical process. In various examples, a PDABL copolymer composition is formed using various polymerization methods known in the art starting from a PDABL composition. Non-limiting examples of polymerizations include condensation polymerizations, addition polymerizations, and the like, which may be catalyzed. In various examples, a copolymer is an elastomer or the like.

In various examples, a PDABL composition can be used to make an article of manufacture. In various examples, an article of manufacture is prepared by forming (e.g., by molding, extrusion, blowing, casting, spinning, or the like) one or more of the PDABL composition(s) of the present disclosure.

In an aspect, the present disclosure provides methods of making PDABL compositions and compositions comprising one or more polymeric chain(s), oligomeric chain(s), or the like, or any combination thereof, the polymeric chain(s) and/or the oligomeric chain(s) each comprising a plurality of dialkyl β-lactone (DABL) repeat units (collectively referred to herein as “PDABL compositions”). In various examples, a method makes PDABL compositions of the present disclosure. Non-limiting examples of methods of making PDABL compositions are described herein.

In various examples, a method of forming a PDABL composition comprises: forming a reaction mixture comprising: one or more dialkyl-β-lactone(s) (DAL(s)), optionally, one or more additional monomers; one or more ring opening polymerization (ROP) initiator(s) and one or more ROP catalyst(s), one or more ROP catalyst-initiator(s), one or more precursor(s) thereof, or any combination thereof; where the PDABL composition is formed. In various examples, the PDABL composition is formed upon ROP of the DAL(s) and, optionally, the non-DAL monomers by the ROP initiator(s) and ROP catalyst(s), ROP catalyst-initiator(s), precursor(s) thereof, or any combination thereof.

In various examples, a method does not comprise a fermentation step. In various examples, the PDABL is not a biopolymer. In various examples, the ROP catalyst(s), ROP initiator(s), ROP catalyst-initiators, or precursors thereof is/are not biologically derived.

In various examples, the DAL(s) is/are chosen from cis DAL(s), trans DAL(s), racemic cis DAL(s), racemic trans DAL(s), mixtures of racemic cis and trans DAL(s), enantioenriched mixtures of at least 90 mol % cis DAL(s), enantioenriched mixtures of at least 90 mol % trans DAL(s), and the like, and any combination thereof.

In various examples, the DAL(s) is/are 2,3-dialkyl-β-lactone(s) (2,3-DAL(s)) (e.g., α,β-dialkyl-β-lactone(s) (α,β-DAL(s)) and the PDABL composition is a poly(2,3-dialkyl-β-lactone) (2,3-PDABL) composition (e.g., a poly(α,β-dialkyl-β-lactone) (α,β-PDABL) composition). In various examples, the DAL(s) has/have the following structure:

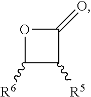

where R⁵ and R⁶ are each chosen, independently, from C₁, C₂, C₃, C₄, C₅, C₆, C₇, and C₈ alkyl groups. In various examples, the DAL(s) is/are 2,3-dimethyl-β-propiolactone(s) (DMPL(s)), and the PDABL composition is a poly(2-methyl-3-hydroxybutyrate) (PMHB) composition comprising a plurality of 2-methyl-3-hydroxybutyrate (MHB) repeat units. In various examples, the DMPL(s) may be chosen from cis DMPL(s), trans DMPL(s), racemic cis DMPL(s), racemic trans DMPL(s), a mixture of racemic cis and trans DMPL(s), an enantioenriched mixture of at least 90 mol % cis DMPL(s), an enantioenriched mixture of at least 90 mol % trans DMPL(s), an enantioenriched mixture of at least 90 mol % cis DMPL(s), and the like, and any combination thereof. Suitable DAL(s) (e.g., 2,3-DAL(s), such as, for example, DMPL(s) and the like) can be obtained commercially and/or prepared by methods known in the art.

ROP is a chain-growth polymerization reaction in which one end of each polymer or oligomer chain carries a reactive center for the addition of cyclic monomers (e.g., DAL(s) and, optionally, non-DAL monomer(s). The resulting polymer or oligomer chain will contain end groups depending on the applied initiator and occurring termination reactions. As used herein, unless otherwise indicated, an ROP initiator is a compound which initiates the ROP reaction. In various examples, a ROP initiator is a cocatalyst. As used herein, unless otherwise indicated, an ROP catalyst is any compound playing an active role in increasing the speed of the ROP reaction, which is generally used with an ROP initiator. A catalyst-initiator is a single compound in which one part of the molecule initiates the ROP reaction while another part catalyzes it.

A method of forming a PDABL composition can use various ROP initiator(s) and ROP catalyst(s), ROP catalyst-initiator(s), precursor(s) thereof, or any combination thereof. Without intending to be bound by any particular theory, it is considered that the ROP initiator(s) and/or ROP catalyst-initiator(s) initiate(s) ROP of the DAL(s) (e.g., 2,3-DAL(s), such as, for example, DMPL(s) and the like) and, optionally, the one or more non-DAL monomers, to form PDABL compositions (e.g., PDABL compositions of the present disclosure).

In various examples, the ROP reaction of the DAL(s) and, optionally, the non-DAL monomers, occurs with retention of stereochemistry, inversion of stereochemistry, monomer specific stereopreference for chain propagation, or the like, or any combination thereof. A ROP catalyst and/or ROP catalyst-initiator, or any combination thereof may exhibit selectivity for a cis-DAL (e.g., cis-DMPL or the like) relative to a trans-DAL (e.g., trans-DMPL or the like). In various examples, a ROP catalyst and/or ROP catalyst-initiator exhibits selectivity for a trans-DAL (e.g., trans-DMPL or the like) in preference to a cis-DAL (e.g., cis-DMPL or the like). Typically, ring-opening rates for a cis-DAL is faster than a trans-DAL, and the rate difference may be dependent on the ROP catalyst and/or ROP catalyst-initiator systems used. An ROP catalyst, and/or ROP catalyst-initiator may exhibit stereospecific chain propagation (e.g., a ROP catalyst, and/or ROP catalyst-initiator may exhibit selectivity for syndiospecific chain propagation of a racemic mixture of DAL(s) relative to isotactic chain propagation or atactic chain propagation). In various examples, an ROP catalyst and/or ROP catalyst-initiator may exhibit stereospecific chain propagation (e.g., syndiospecific chain propagation of a racemic mixture of DAL(s) (e.g., a racemic mixture of DMPL(s) or the like) and may exhibit selectivity for cis-DAL(s) (e.g., cis-DMPL(s) or the like) relative to a trans-DAL(s) (e.g., trans-DMPL(s) or the like).

In various examples, the ROP catalyst-initiator(s) is/are chosen from organic salt(s), carbene(s) (e.g., N-heterocyclic carbenes or the like), metal alkoxide(s) and/or aryloxide(s), (multidentate ligand) metal alkoxide complex(es), metal aryloxide complex(es), and the like, and any combination thereof, and where the metal is, independently at each occurrence, chosen from main group metals, transition metals and rare-earth metals. In various examples, the organic salt(s) is/are chosen from imidazolium salt(s), aminophosphonium salt(s), diphosphazenium salt(s), ammonium salt(s), and the like, and any combination thereof; and/or the alkoxide(s) and/or aryloxide(s) is/are chosen from C₁-C₂₀ alkoxide(s), C₁-C₂₀ aryloxide(s), and the like, and any combination thereof.

In various examples, the ROP catalyst-initiator(s) is/are chosen from: yttrium (III) tris(isopropoxide)(Y(OⁱPr)₃); magnesium (II) benzhydrol (Mg(BH)₂); zinc (II) benzhydrol (Zn(BH)₂); magnesium (II) phenoxide isopropoxide ([L]Mg(OⁱPr) or a dimer thereof), where L is a phenoxide ligand having the following structure:

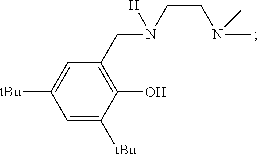 [SMILES: CN(C)CCNCc1cc(C(C)(C)C)cc(C(C)(C)C)c1O]

zinc (II) phenoxide isopropoxide ([L]Zn(OⁱPr) or a dimer thereof), where L is a phenoxide ligand, independently at each occurrence, chosen from:

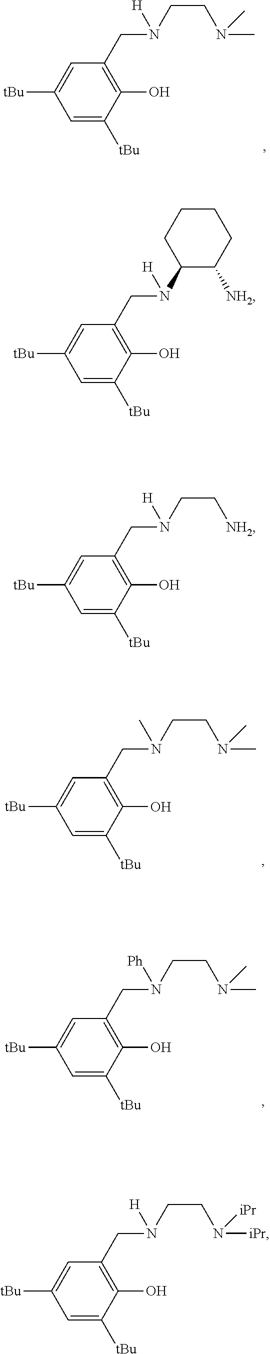 [SMILES: CN(C)CCNCc1cc(C(C)(C)C)cc(C(C)(C)C)c1O ; CC(C)(C)c1cc(CN[C@H]2CCCC[C@@H]2N)c(O)c(C(C)(C)C)c1 ; CC(C)(C)c1cc(CNCCN)c(O)c(C(C)(C)C)c1 ; CN(C)CCN(C)Cc1cc(C(C)(C)C)cc(C(C)(C)C)c1O ; CN(C)CCN(Cc1cc(C(C)(C)C)cc(C(C)(C)C)c1O)c1ccccc1 ; CC(C)N(CCNCc1cc(C(C)(C)C)cc(C(C)(C)C)c1O)C(C)C]

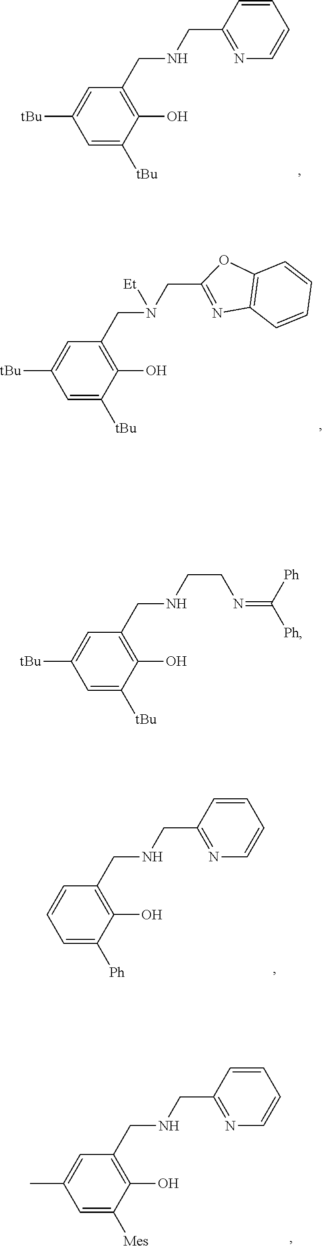

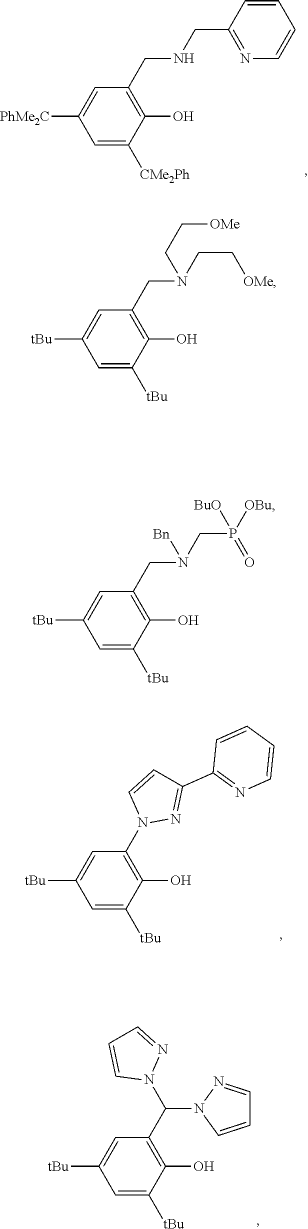

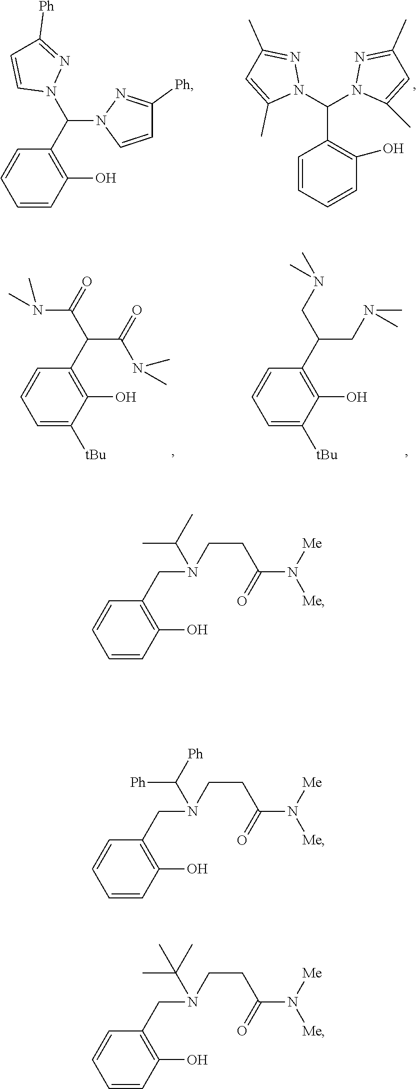

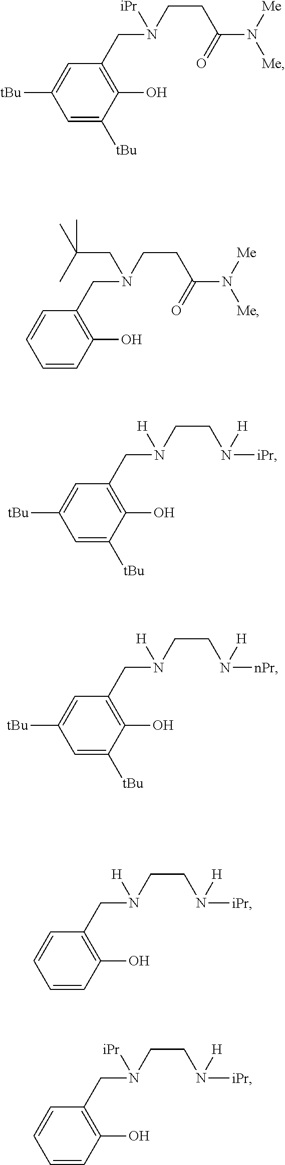

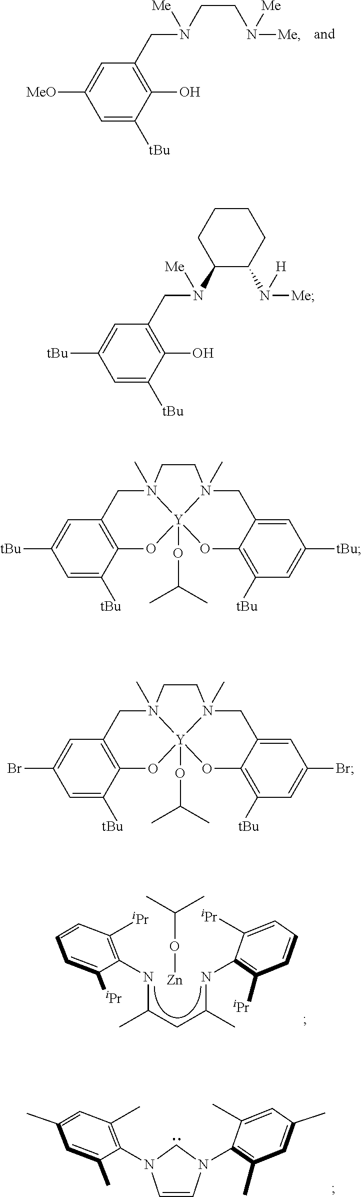

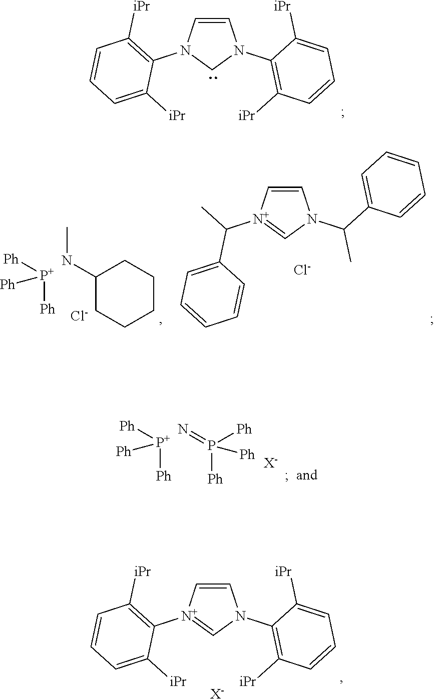

where X⁻ is, independently at each occurrence, chosen from RCO₂⁻, Cl⁻, HCO₃⁻, and N₃⁻, and the like, and where R is, independently at each occurrence, chosen from hydrogen group, aliphatic groups (e.g., methyl, t-butyl, adamantyl, and the like), aryl groups (e.g., phenyl, and the like), and the like; and the like; and any combination thereof.

In various examples, the reaction mixture comprises one or more ROP initiator(s) and one or more ROP catalyst(s), one or more ROP catalyst-initiator(s), precursor(s) thereof, or any combination thereof. In various examples, the ROP initiator(s) is/are chosen from aliphatic alcohol initiator(s), aromatic alcohol initiator(s), aliphatic carboxylic acid initiator(s), aromatic carboxylic acid initiator(s), and the like, and any combination thereof. In various examples, the ROP initiator(s) is/are chosen from succinic acid, benzoic acid, benzyl alcohol, and the like, and any combination thereof.

In various examples, the ROP catalyst(s) is/are chosen from ROP catalyst-initiator(s). In various examples, the ROP catalyst(s) is/are chosen from N-heterocyclic carbenes (e.g.,

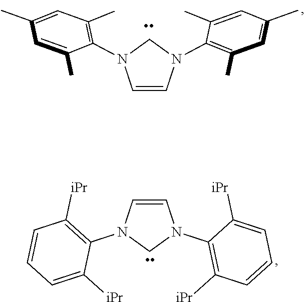

and the like, and any combination thereof), diphosphazenium salt(s) (e.g.,

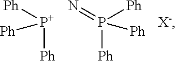

where X⁻ is, independently at each occurrence, chosen from RCO₂⁻, Cl⁻, HCO₃⁻, and N₃⁻, and the like, and where R is, independently at each occurrence, chosen from hydrogen group, aliphatic groups (e.g., methyl, t-butyl, adamantyl, and the like), aryl groups (e.g., phenyl, and the like), and the like), and the like, and any combination thereof.

In various examples, the reaction mixture comprises one or more ROP catalyst-initiator(s) chosen from carbene(s), diphosphazenium salt(s), and the like, and any combination thereof, and one or more ROP initiator(s) chosen from aliphatic carboxylic acid initiator(s), aromatic carboxylic acid initiator(s), aliphatic alcohol initiator(s), aromatic alcohol initiator(s), and any combination thereof.

In various examples, one or more or all of the ROP initiator(s) and/or ROP catalyst(s), or ROP catalyst-initiator(s) is/are formed and isolated prior to the forming of the reaction mixture. In various examples, one or more or all of the ROP initiator(s) and/or ROP catalyst(s), or ROP catalyst-initiator(s) is/are formed by precursor(s) thereof in situ in the reaction mixture.

In various examples, the reaction mixture further comprises one or more non-DAL monomer(s). In various examples, the non-DAL monomer(s) is/are, independently at each occurrence, chosen from substituted and unsubstituted β-, γ-, δ-, and ω-lactone(s), cyclic ether(s), cyclic carbonate(s), cyclic carbamate(s), structural analogs thereof, and the like, and any combination thereof. In various examples, the non-DAL monomer(s) is/are, independently at each occurrence, chosen from substituted and unsubstituted β-propiolactone, β-butyrolactone, β-valerolactone, β-caprolactone, lactide, glycolides, structural analogs thereof, and the like, and any combination thereof. A method of forming a PDABL composition can use various amounts of the non-DAL monomer(s). In various examples, the non-DAL monomer(s) is/are added to the reaction mixture at about 1 mol % to about 50 mol %, including all 0.1 mol % values and ranges therebetween (e.g., about 5 to about 50 mol % or about 10 to about 50 mol %), based on the total moles of monomers.

A method of forming a PDABL composition can use various amounts of ROP initiator(s) and ROP catalyst(s), ROP catalyst-initiator(s), precursor(s) thereof, or any combination thereof. In various examples, the reaction mixture comprises from about 0.01 mol % to about 1 mol % of the ROP initiator(s) and the ROP catalyst(s), the ROP catalyst-initiators(s), precursor(s) thereof, or any combination thereof, based on the total moles of the DAL(s), the non-DAL monomer(s), and the ROP initiator(s) and the ROP catalyst(s), the ROP catalyst-initiators(s), precursor(s) thereof, or any combination thereof.

A method of forming a PDABL composition can use various additional components. In various examples, the DAL(s) (e.g., DMPL(s) or the like) and the ROP initiator(s) and the ROP catalyst(s), the ROP catalyst-initiator(s), the precursor(s) thereof, or any combination thereof, is/are contacted with in one or more solvent(s). In various examples, the reaction mixture comprises the DAL(s) (e.g., DMPL(s) or the like), the ROP initiator(s) and the ROP catalyst(s), the ROP catalyst-initiators(s), the precursor(s) thereof, or any combination thereof, optionally, the non-DAL monomer(s) and, optionally, the one or more solvent(s). In various examples, the solvent(s) are chosen from organic solvents, and the like, and any combination thereof. In various examples, the organic solvent(s) is/are chosen from polar aprotic solvents, ether solvents, aromatic solvents, chlorinated solvents, lactone solvents, and the like, and any combination thereof. Non-limiting examples of organic solvents include tetrahydrofuran (THF), benzene, toluene, diethylether, dimethoxyethane (DME), chlorinated solvents (e.g., chlorobenzene, dichloromethane (DCM), chloroform, and the like), γ-valerolactone, and the like, and any combination thereof.

An ROP reaction can be performed under various reaction conditions. A reaction can comprise one or more steps and each step can be performed under the same or different reaction conditions as other steps. A reaction can be carried out at various temperatures. In various examples, a reaction is carried out at room temperature (e.g., from about 20° C. to about 22° C., including all 0.1° C. values and ranges therebetween), below room temperature (e.g., at about 20° C. or below, such as for example, from about −50° C. to about 20° C., including all 0.1° C. values and ranges therebetween) (e.g., about −50° C. to about 0° C., or about −10° C. to about 20° C.), above room temperature (e.g., at a temperature up to or about a boiling point of the solvent(s), if present) (e.g., at about 20° C. to about 200° C., including all 0.1° C. values and ranges therebetween) (e.g., about 20° C. to about 100° C., about 100° C. to about 110° C., about 100° C. to about 120° C., or about 100° C. to about 150° C.), or the like, or any combination thereof (e.g., where each step is performed at a different temperature as other steps). In various examples, the ROP reaction is performed at a temperature of about 0° C. to about 150° C.

A reaction can be carried out for various times. The reaction time can depend on factors such as, for example, temperature, pressure, presence and/or efficiency of a catalyst, presence and/or intensity of an applied energy source, mixing (e.g., stirring, or the like), or the like, or any combination thereof. In various examples, reaction times range from about seconds (e.g., two seconds) to greater than about 200 hours, including all integer second values and ranges therebetween (e.g., from about 1 minute to about 150 hours, including all integer second values and ranges therebetween) (e.g., about 10 minutes, about 1 hour, about 12 hours, about 24 hours, about 120 hours, or about 150 hours), or the like, or any combination thereof (e.g., where each step is performed at a different time as other steps). In various examples, the ROP reaction is performed for about 1 minute to about 48 hours (e.g., about 1 minute to about 24 hours).

A reaction can be carried out at various pressures. In various examples, a reaction is carried out at atmospheric pressure (e.g., 1 standard atmosphere (atm) at sea level), at greater than atmospheric pressure (e.g. heating in a sealed pressurized reaction vessel and the like), at below atmospheric pressure (e.g., under vacuum (e.g., from about 1 mTorr or less to about 100 mTorr or less, including all 0.1 mTorr values and ranges therebetween) (e.g., about 100 mTorr or less, about 50 mTorr or less, about 10 mTorr or less, or about 1 mTorr or less) and the like), or the like, or any combination thereof (e.g., where each step is performed at a different pressure as other steps).

A method may have a desirable ROP polymerization conversion. A ROP conversion can be measured by methods known in the art. In various examples, a ROP conversion of a PDABL composition is determined (e.g., measured) by ¹H nuclear magnetic resonance (NMR), ¹³C NMR (e.g., quantitative ¹³C NMR by peak integration), or the like, or any combination thereof. In various examples, a method has a ROP conversion of a PDABL composition of about 90 mol % to about 100 mol %, including all 0.1 ROP conversion values and ranges therebetween. In various examples, the DAL(s) and, optionally, the non-DAL monomer(s) is/are contacted with the ROP catalyst(s) in any order and/or for any degree of polymer conversion. In various examples, the DAL(s) and, optionally, the non-DAL monomer(s) are all contacted with the ROP catalyst(s) at the same time. In various examples, one or more of the DAL(s) is/are contacted with the ROP catalyst(s) for any degree of polymer conversion prior to the contact of any of the remaining one or more DAL(s), one or more non-DAL monomer(s), or the like, or any combination thereof, with the ROP catalyst(s). In various examples, one or more of the non-DAL monomer(s) is/are contacted with the one or more ROP catalyst(s) for any degree(s) of polymer conversion prior to the contact of any of the remaining DAL(s), one or more non-DAL monomer(s), or the like, or any combination thereof, with the ROP catalyst(s). In various examples, the one or more DAL(s) are contacted with the one or more ROP catalyst(s) for any degree of polymer conversion prior to the contact of the one or more non-DAL monomer(s) with the one or more ROP catalyst(s). In various examples, the non-DAL monomer(s) is/are contacted with the ROP catalyst(s) for any degree of polymer conversion prior to the contact of the DAL(s) with the one or more ring-opening polymerization catalyst(s).

A method of forming a PDABL composition can comprise various additional steps. In various examples, a method further comprises isolation of at least a portion of, substantially all, or all of the composition. In various examples, a method further comprises using a PDABL composition made by the method as a starting material in another functionalization and/or polymerization reaction. In various examples, a method further comprises forming a copolymer of the PDABL composition made by the method using various polymerization methods known in the art. Non-limiting examples of polymerizations include condensation polymerizations, addition polymerizations, and the like, which may be catalyzed. In various examples, a copolymer is an elastomer or the like. In various examples, a method further comprises forming an article of manufacture (e.g., by molding, extrusion, blowing, casting, or spinning one or more of the PDABL composition(s)). In various examples, a method further comprises any combination of these additional steps.

In an aspect, the present disclosure provides uses of PDABL compositions (e.g., of the present disclosure or prepared by a method of the present disclosure). Non-limiting examples of uses PDABL compositions of the present disclosure and/or prepared by a method of the present disclosure are described herein.

PDABL compositions can be used in and/or to form articles of manufacture. In various examples, an article of manufacture comprises one or more PDABL compositions. In various examples, an article of manufacture is prepared by forming (e.g., by molding, extruding, blowing, casting, spinning, or the like) one or more PDABL composition of the present disclosure or prepared by a method of the present disclosure). In various examples, an article of manufacture is a molded article, an extruded article, a blown article, a cast article, a spun article, or the like. In various examples, an article of manufacture is in the form of a monolith, a coating, a sheet, a film, a fiber, a solid article, a hollow article, a foam, a composite, or the like. In various examples, an article of manufacture is a packaging article, a single-use article, a sports article, a biomedical article, an agricultural article, an automotive article, an electronic article, or the like. In various examples, packaging article is a film, a wrapping, a sheet, a textile, a net, a bag, a container, a tub, a closure, a cap, a handle, a dispenser, a filler, a protector, a pad, a fastener, or the like. In various examples, a single-use article is a bag, a container, a dispenser, a cup, a bottle, a plate, cutlery, a straw, or the like. In various examples, a sports article is fishing line, or the like. In various examples, a biomedical article is a drug delivery article, a wound closure article, a wound dressing article, a surgical suture, a medical implant, a tissue engineering construct, or the like. In various examples, an agricultural article is a film, a wrapping, a sheet, a textile, a net, a twine, a string, clips, wires, stakes, a bag, a container, a tub, a closure, a cap, a handle, a dispenser, a filler, a protector, a pad, a fastener, a bottle, a lid, a pot, mulch, or the like. In various examples, an article of manufacture is degradable (or biodegradable). In various examples, an article of manufacture is degradable (or biodegradable) within bodily tissues, fluids, or organs; soil, compost, or other organic matter; fresh water or salt water; landfills, or the like, or any combination thereof. In various examples, an article of manufacture is at least partially or completely degradable (or biodegradable) by microbial degradation, hydrolysis, photodegradation, or the like, or any combination thereof. In various examples, an article of manufacture is at least partially or completely degradable (or biodegradable) under aerobic conditions, anaerobic conditions, or the like, or any combination thereof.

In an aspect, the present disclosure provides methods of depolymerization of PDABL compositions (e.g., of the present disclosure or prepared by a method of the present disclosure). Non-limiting examples of methods of depolymerization of PDABL compositions of the present disclosure and/or prepared by a method of the present disclosure are described herein.

In various examples, a depolymerization method comprises: forming a depolymerization mixture comprising: one or more PDABL composition(s) of the present disclosure and/or prepared by a method of the present disclosure; and one or more depolymerization catalyst(s); and heating the depolymerization mixture, thereby forming one or more depolymerization product(s). In various examples, the reaction mixture does not comprise a solvent. In various examples, the reaction mixture is a melt comprising only the PDABL composition(s) and the depolymerization catalyst(s).

In various examples, the PDABL composition(s) is/are one or more article(s) of manufacture, and where the article(s) of manufacture comprise(s) the one or more PDABL composition(s) of the present disclosure and/or prepared by a method of the present disclosure. In various examples, the PDABL composition(s) comprise(s) only the DABL repeat unit(s). In various examples, the PDABL composition(s) comprise(s) only DABL repeat unit(s) (e.g., only MHB repeat units or the like). In various examples, the PDABL composition(s) comprise only the cis or only the trans DABL repeat unit(s) (e.g., only the cis or only the trans MHB repeat unit(s)). In various examples, the PDABL composition(s) comprise about 50 mol % to about 100 mol % of the cis DABL repeat unit(s) (e.g., the cis MHB repeat unit(s) or the like), including all 0.1 mol % value and ranges therebetween (e.g., about 50 mol % to about 95 mol %, or about 70 mol % to about 95 mol % cis DABL repeat unit(s)). In various examples, the PDABL composition(s) comprise(s) about 60 mol % to about 95 mol %, including all 0.1 mol % values and ranges therebetween, of the racemo (r) DABL repeat unit(s) (e.g., the raceme (r) MHB repeat unit(s)) (e.g., about 70 mol % to about 95 mol % the racemo (r) DABL repeat unit(s)). In various examples, the PDABL composition(s) comprise(s) a number average molecular weight of from about 75 kDa to about 300 kDa, including all 0.1 kDa values and ranges therebetween.

In various examples, the depolymerization catalyst(s) is/are chosen from non-metal oxide(s), group II metal oxide(s), group II metal aliphatic carboxylate(s), group II metal aromatic carboxylate(s), aliphatic organic acid(s), aromatic organic acid(s), aliphatic organic base(s), aromatic organic base(s), salts thereof, and the like, and any combination thereof. In various examples, the metal oxide catalyst(s) is/are chosen from magnesium oxide, magnesium tiglate, silica gel, sand, p-toluenesulfonic acid, 4-dimethylaminopyridine, and the like, and any combination thereof. In various examples the depolymerization catalyst(s) is/are solid acid catalyst(s). In various examples, the depolymerization mixture comprises about 1 wt. % to about 20 wt. %, including all 0.1 wt. % values and ranges therebetween, of the depolymerization catalyst(s), based on the total weight of the depolymerization mixture (e.g., about 1 wt. % to about 10 wt. % of the depolymerization catalyst(s)).

A depolymerization reaction can be performed under various reaction conditions. A depolymerization reaction can comprise one or more steps and each step can be performed under the same or different reaction conditions as other steps. A depolymerization reaction can be carried out at various temperatures. In various examples, a reaction is carried out at room temperature (e.g., from about 20° C. to about 25° C., including all 0.1° C. values and ranges therebetween), below room temperature (e.g., at about 0° C. or below, such as for example, from about −50° C. to about 0° C., including all 0.1° C. values and ranges therebetween) (e.g., about −20° C. to about 0° C.), above room temperature (e.g., at a temperature up to or about a boiling point of the solvent(s), if present) (e.g., at about 100° C. or above, e.g. from about 100° C. to about 400° C., including all 0.1° C. values and ranges therebetween) (e.g., about 100° C., about 200° C., about 300° C., about 400° C., or about 500° C.), or the like, or any combination thereof (e.g., where each step is performed at a different temperature as other steps).

A depolymerization reaction can be carried out at various pressures. In various examples, a depolymerization reaction is carried out at atmospheric pressure (e.g., 1 standard atmosphere (atm) at sea level), at greater than atmospheric pressure (e.g. heating in a sealed pressurized reaction vessel and the like), at below atmospheric pressure (e.g., under vacuum (e.g., from about 1 mTorr or less to about 100 mTorr or less, including all 0.1 mTorr values and ranges therebetween) (e.g., about 100 mTorr or less, about 50 mTorr or less, about 10 mTorr or less, or about 1 mTorr or less) and the like), or the like, or any combination thereof (e.g., where each step is performed at a different pressure as other steps).

A depolymerization reaction can be carried out for various times. The depolymerization reaction time can depend on factors such as, for example, temperature, pressure, presence and/or efficiency of a catalyst, presence and/or intensity of an applied energy source, mixing (e.g., stirring, or the like), or the like, or any combination thereof. In various examples, depolymerization reaction times range from about seconds (e.g., two seconds) to greater than about 200 hours, including all integer second values and ranges therebetween (e.g., from about 1 minute to about 150 hours, including all integer second values and ranges therebetween) (e.g., about 10 minutes, about 1 hour, about 12 hours, about 24 hours, about 120 hours, or about 150 hours), or the like, or any combination thereof (e.g., where each step is performed at a different time as other steps).

In various examples, the depolymerization mixture is heated to a temperature below the decomposition temperature of the depolymerization catalyst(s) and the PDABL composition(s)/PDABLs. In various examples, the depolymerization mixture is a melt comprising only the depolymerization catalyst(s) and the PDABL composition(s)/PDABLs. In various examples, the depolymerization mixture is heated according to one or more or all of the following: at a temperature of from about 190° C. to about 220° C., including all 0.1° C. values and ranges therebetween, for a time of from about 1 hour (h=hour(s)) to about 12 h including all 1 second (sec) values and ranges therebetween; or under inert conditions. In various examples, the depolymerization product(s) is/are alpha,beta-alkenoic acid(s) (such as, for example, tiglic acid and the like, or a combination thereof). In various examples, the method further comprising isolating and, optionally, purifying, the depolymerization product(s).

The following Statements describe various examples of methods, products and systems of the present disclosure and are not intended to be in any way limiting:

Statement 1. A poly(dialkyl β-lactone) (PDABL) composition or a composition (e.g., a PDABL composition) comprising one or more polymeric chain(s), oligomeric chain(s), or the like, or any combination thereof, the polymeric chain(s) and/or the oligomeric chain(s) each comprising a plurality of dialkyl β-lactone (DABL) repeat units.
Statement 2. A composition according to Statement 1, where at least a portion of or all of the one or more polymeric chain(s), oligomeric chain(s), or the like, or any combination thereof, independently, at least partially or completely, comprise cis and/or trans DABL repeat units (e.g., cis and/or trans MHB repeat units and the like).
Statement 3. A composition according to Statement 1 or 2, where at least a portion of or all of the one or more polymeric chain(s), oligomeric chain(s), or the like, or any combination thereof, independently, at least partially or completely, comprise randomly oriented and/or stereoregular DABL diads (e.g., MHB diads and the like).
Statement 4. A composition according to any of the preceding Statements, where the one or more polymeric chain(s), oligomeric chain(s), or the like, or any combination thereof, individually, at least partially or completely, comprise less than 100 mol % DABL repeat units (e.g., MHB repeat units and the like).
Statement 5. A composition according to any of the preceding Statements, where the one or more polymeric and/or oligomeric chains, individually, at least partially or completely, comprise crystalline and/or amorphous domains.
Statement 6. A composition according to any of the preceding Statements, where the composition is in the form of a monolith, a film, a fiber, a flake, a pellet, a powder, a granule, a particle, a bead, a bar, a liquid, a solution, an emulsion, or the like, or any combination thereof.
Statement 7. A composition according to any of the preceding Statements, where the composition exhibits or has one or more or all of the following:
A melting temperature (Tm), which may be measured by differential scanning calorimetry (DSC), of about 120 to about 250° C., including all 0.1° C. values and ranges therebetween.
about An enthalpy of crystallization (ΔHc), which may be measured by DSC, of 10 to 60 J/g, including all 0.1 J/g values and ranges therebetween.
A decomposition temperature (Td), which may be measured by thermogravometric analysis (TGA), of about 240 to about 350° C. (e.g., about 260° C. to about 290° C.) including all 0.1° C. values and ranges therebetween.
A crystallization temperature of about 10° C. to about 170° C., including all 0.1° C. values and ranges therebetween.
   8. A composition according to any of the preceding Statements, where the composition exhibits or has an elongation at break of about 200% or more, about 300% or more, about 400% or more, about 500% or more, about 600% or more, about 700% or more, about 800% or more, about 900% or more, or about 1000% or more (e.g., about 300% to about 1200%, including all integer % values and ranges therebetween), and/or a tensile strength of about 5 MPa or more, about 10 MPa or more, about 20 MPa or more, about 25 MPa or more, about 30 MPa or more, about 35 MPa or more, about 40 MPa or more, about 45 MPa or more, or about 50 MPa or more (e.g., about 5 to about 50 MPa, including all 0.1 MPa values and ranges therebetween).
   9. A composition of any of the preceding Statements, where the one or more polymeric and/or oligomeric chain(s), independently, are at least partially or completely degradable (or biodegradable).
   10. A homopolyester or copolyester comprising 50 to 100 mol % of one or more repeat units having the formula

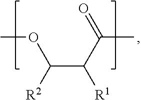

where R¹ and R² are each independently at each occurrence a hydrocarbyl group comprising 1 to 8 carbon atoms, and
0 to 50 mol % repeat units having the formula

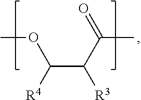

where R³ and R⁴ are each independently at each occurrence a hydrogen group or a hydrocarbyl group comprising 1 to 10 carbon atoms, with the proviso that at least one of R³ and R⁴ independently is a hydrogen.
11. A homopolyester or copolyester according to Statement 10, produced by the polymerization of one or more 2,3-dialkyl-β-propiolactone monomer(s) and optionally with one or more monosubstituted alkyl-β-propiolactone monomer(s).
12. A homopolyester or copolyester according to Statement 10 or 11, where the polymer or copolymer comprises a terminal hydrogen group, a hydroxyl group (—OH), a carboxylic acid group (—COOH), or any combination thereof.
13. A homopolyester or copolyester according to any of Statements 10-12, where the polymer or copolymer has a number average molecular weight of about 1,000 to about 500,000 g/mol.
14. A homopolyester or copolyester according to any of Statements 10-13, where the polymer or copolymer has a weight average molecular weight of about 1,000 to about 500,000 g/mol.
15. A homopolyester or copolyester according to any of Statements 10-14, comprising repeat units where R¹ and R² are independently at each occurrence chosen from methyl group, ethyl group, n-propyl group, isopropyl groups, butyl groups, pentyl groups, hexyl groups, heptyl groups, and octyl groups.
16. A homopolyester or copolyester according to any of Statements 10-15, the polymer or copolymer comprising repeat units where the R¹ and R² groups comprise a geometric isomeric configuration of cis-cis, cis-trans or trans-trans.
17. A homopolyester or copolyester according to any of Statements 10-16, the polymer or copolymer comprising a polymer microstructure that is atactic, isotactic or syndiotactic with respect to the stereochemical sequence along the polymer backbone.
18. A homopolyester or copolyester according to any of Statements 10-17, where the repeat units are arranged by composition or by tacticity such that blocks of composition and/or tacticity are formed within the copolymer.
19. A method to forming an article of manufacture by forming (e.g., by molding, extrusion, blowing, casting, spinning, or the like) one or more of the homopolyester(s) and/or copolyester(s) of any of Statements 10-18.
20. A method of forming a PDABL composition or a PDABL comprising one or more polymeric chain(s), oligomeric chain(s), or the like, or any combination thereof, the polymeric chain(s) and/or the oligomeric chain(s) each comprising a plurality of β-lactone (BL) repeat units (e.g., a composition/polymer of the present disclosure or a composition/polymer according to any of Statements 1-9), or a homopolymer or copolymer (e.g., a homopolymer or copolymer of the present disclosure or a homopolymer or copolymer according to any of Statements 10-19), the method comprising contacting one or more 2,3-dialkyl-β-lactone(s) (DAL(s)) (at least one of which or all of which may be 2,3-dimethyl-β-propiolactone(s) (DMPL(s))), and, optionally, one or more additional monomer(s) (one or more or all of which may be non-DAL monomers, such as, for example, other lactones, and the like)), with one or more ring opening polymerization catalyst(s), where the one or more additional monomer(s) are capable of being polymerized by the one or more ring opening polymerization catalyst(s), and
where the composition is formed.
21. A method of Statement 20, further comprising forming a copolymer comprising one or more composition(s) and/or polymer(s) of Statement 10.
22. A method according to Statement 20 or 21, further comprising forming an article of manufacture by forming (e.g., by molding, extrusion, blowing, casting, spinning, or the like) one or more of the composition(s).
23. An article of manufacture comprising one or more composition(s) of the present disclosure (e.g., one or more composition(s) of any of Statements 1-9, one or more composition(s) made by a method of any of Statements 20-22, or a homopolymer or copolymer of the present disclosure (e.g., a homopolymer or copolymer according to any of Statements 10-19), or the like, or any combination thereof.
24. An article of manufacture according to Statement 23, where the article of manufacture is in the form of a monolith, a coating, a sheet, a film, a fiber, a solid article, a hollow article, a foam, a composite, or the like.
25. An article of manufacture according to Statement 23 or 24, where the article of manufacture is a packaging article, a single-use article, a sports article, a biomedical article, an agricultural article, an automotive article, an electronic article, or the like.
26. An article of manufacture according to Statement 25, where the packaging article is a film, a wrapping, a sheet, a textile, a net, a bag, a container, a tub, a closure, a cap, a handle, a dispenser, a filler, a protector, a pad, a fastener, or the like.
27. An article of manufacture according to Statement 25, where the single-use article is a bag, a container, a dispenser, a cup, a bottle, a plate, cutlery, a straw, or the like.
28. An article of manufacture according to Statement 25, where the sports article is fishing line, or the like.
29. An article of manufacture according to Statement 25, where the biomedical article is a drug delivery article, a wound closure article, a wound dressing article, a surgical suture, a medical implant, a tissue engineering construct, or the like.
30. An article of manufacture according to Statement 25, where the agricultural article is a film, a wrapping, a sheet, a textile, a net, a twine, a string, clips, wires, stakes, a bag, a container, a tub, a closure, a cap, a handle, a dispenser, a filler, a protector, a pad, a fastener, a bottle, a lid, a pot, mulch, or the like.
31. An article of manufacture according to any of Statements 23-30, where the article of manufacture is degradable (or biodegradable).

The steps of the methods described in the various examples disclosed herein are sufficient to carry out the methods of the present disclosure. Thus, in an example, a method consists essentially of a combination of the steps of the methods disclosed herein. In another example, a method consists of such steps.

The following examples are presented to illustrate the present disclosure. The examples are not intended to be limiting in any manner.

### EXAMPLE 1

The following is an example of PDABL compositions of the present disclosure, methods of making same, and uses same.

Semicrystalline polyesters with good thermal properties were prepared by ring-opening polymerization of 2,3-dimethyl-β-propiolactone to form poly(2-methyl-3-hydroxybutyrate) (PMHB). These polymers had high melting points (Tₘ>120° C.) and decomposition points (T_{d}>260° C.), making them suitable for use as thermoplastics. The properties of PMHB were influenced by the microstructure of the polymer, including the relative orientation of the two methyl groups in the repeating unit (cis vs trans) and the relative stereochemistry of the repeating units within the chain (atactic, isotactic and syndiotactic.) Different PMHB microstructures were achieved by using different catalysts and/or isomeric forms of 2,3-dimethyl-β-propiolactone. Notably, semi-crystalline polymers with good mechanical properties were prepared from racemic cis or trans 2,3-dimethyl-β-propiolactone or mixtures of the cis or trans isomers; this will facilitate the use of an equilibrium mixture of cis- and trans-2-butene to be used as a feedstock.

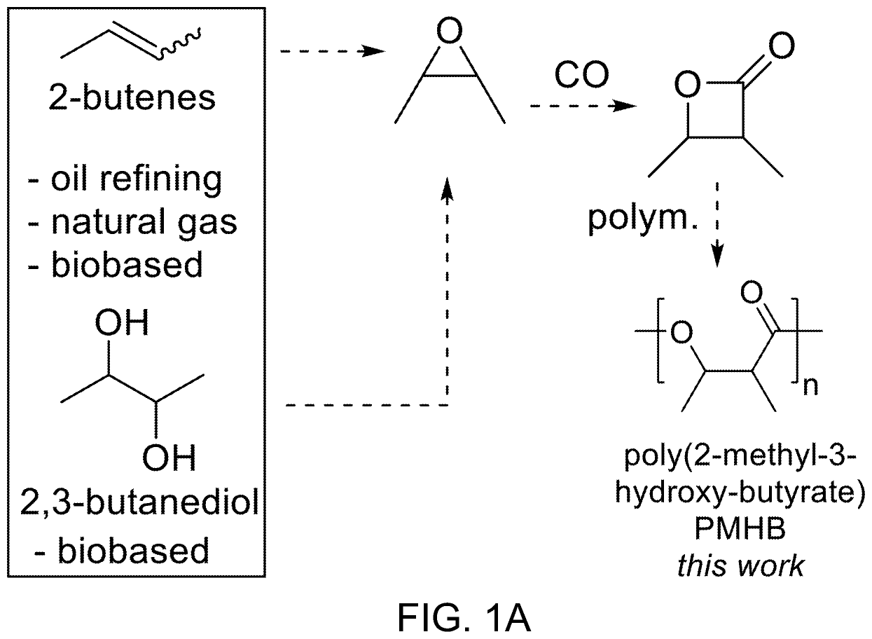

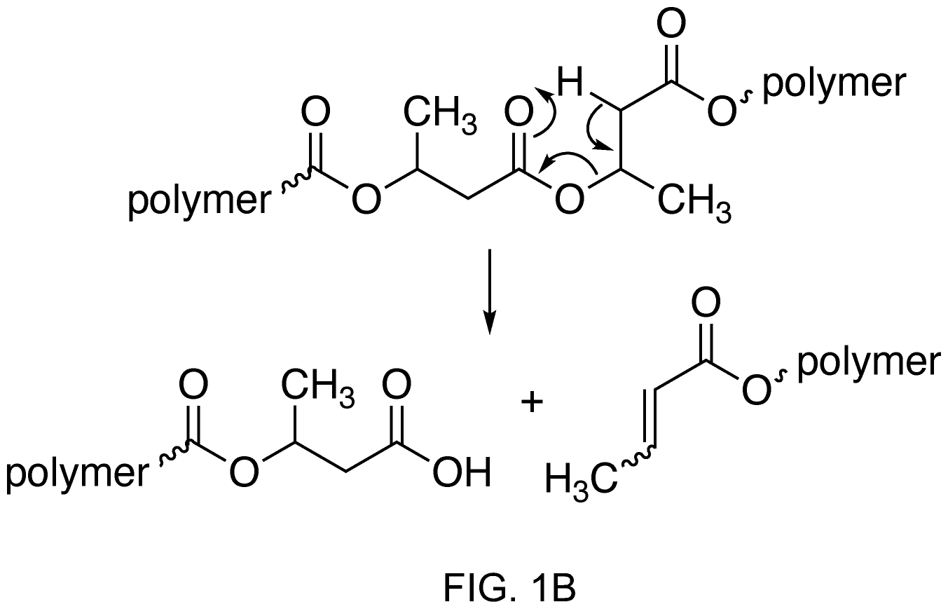

A new class of aliphatic polyesters are described, namely poly(2-methyl-3-hydroxybutyrate) (PMHB) that can be accessed through ring opening polymerization of the corresponding β-lactone monomers (FIG. 1A). These polyesters have high melting points and may provide biodegradable or readily recyclable alternatives to polyethylene and polypropylene. They may be derived from the C₄ feedstock butene, which may be sourced from either fossil fuel or biomass sources. These monomers may be prepared from carbonylation reaction of 2-butene oxide, which can be produced either by epoxidation of 2-butene, a byproduct of the oil refining process, or dehydration of 2,3-butanediol, a biorenewable resource produced by bacteria and/or from biomass. The ready availability of the feedstocks from multiple sources is advantageous for large scale production of these polyesters. The properties may be tuned by varying the stereochemistry of the lactone monomer and/or the choice of catalyst. In addition, useful materials were obtained from the racemic lactone monomers, including mixtures of racemic cis- and trans-2,3-dimethyl-β-propiolactone.

Currently, the commercial polyester R-poly(3-hydroxybutyrate) (R-PHB) has a similar melting point as several isomers of PMHB. However, R-PHB is produced by bacteria and is expensive to produce, and is very brittle unless modified by the presence of comonomers. In addition, R-PHB (decomposition temperature (T_{d})=180° C.) is substantially less thermally stable than PMHB (T_{d}=267-287° C.). This limits its applications as a thermoplastic.

The semicrystalline polymers of the present disclosure have useful thermal properties which distinguish them from the commercial aliphatic polyesters PHB and PLA. The high melting point of several microstructures of PMHB (>120° C.) makes it suitable for applications for which PLA cannot be used. Likewise, cis-atactic PMHB, cis-syndiotactic PMHB and trans-syndiotactic PMHB have a Tₘ similar to R-PHB but may be prepared from racemic lactone. In contrast, isotactic PHB must be prepared from enantioenriched lactone or the diolide dimer or produced by bacteria. PMHB may be made from a readily available feedstock, 2-butene (FIG. 1A); polymers with useful tensile properties may even be made from mixtures of the cis- and trans-lactone. For this reason, they may ultimately be more economical to produce than other reported polyesters and polythioesters.

In addition to its bioproduction, PHB can be produced by ring-opening polymerization of β-butyrolactone. Atactic, isotactic, and syndiotactic microstructures have been reported, as well as block and tapered copolymers. Because of the useful properties and range of PHB microstructures that can be achieved by ring-opening polymerization of β-butyrolactone, the effect of adding an additional methyl substituent at the C3 position of the lactone was investigated. This modification was predicted to increase the thermal stability of the resultant polymers by partially shutting down a thermal decomposition pathway (FIG. 1B) which proceeds through a concerted, six-membered-ring transition state. The addition of one or two alkyl substituents adjacent to the carbonyl inhibits this mode of decomposition, resulting in significantly higher decomposition temperatures. Although the effect of adding small amounts of alkyl substituents to PHB, including methyls, at the carbon adjacent to the carbonyl has been reported, the homopolymer of 2,3-dimethyl-β-propiolactone, poly(2-methyl-3-hydroxybutyrate (PMHB)), has not been reported.

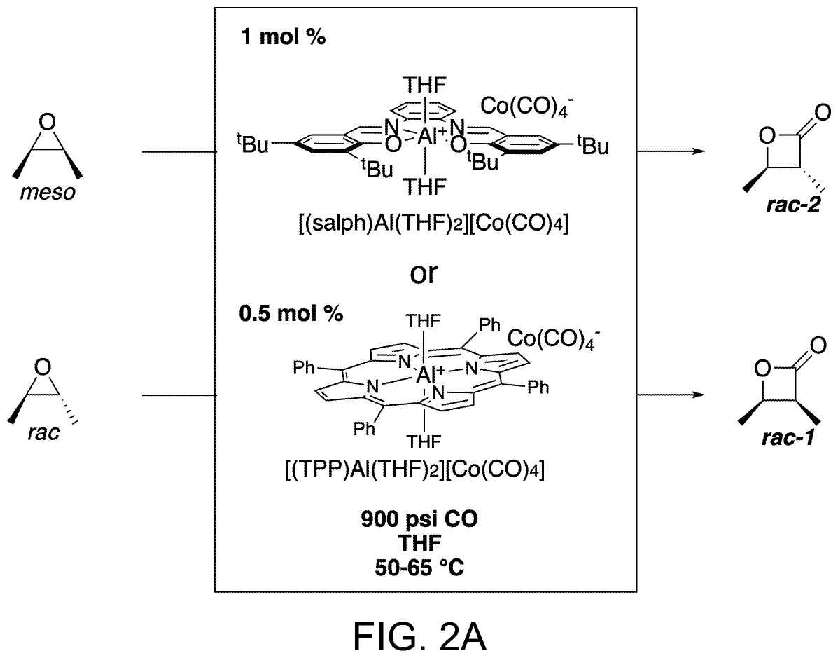

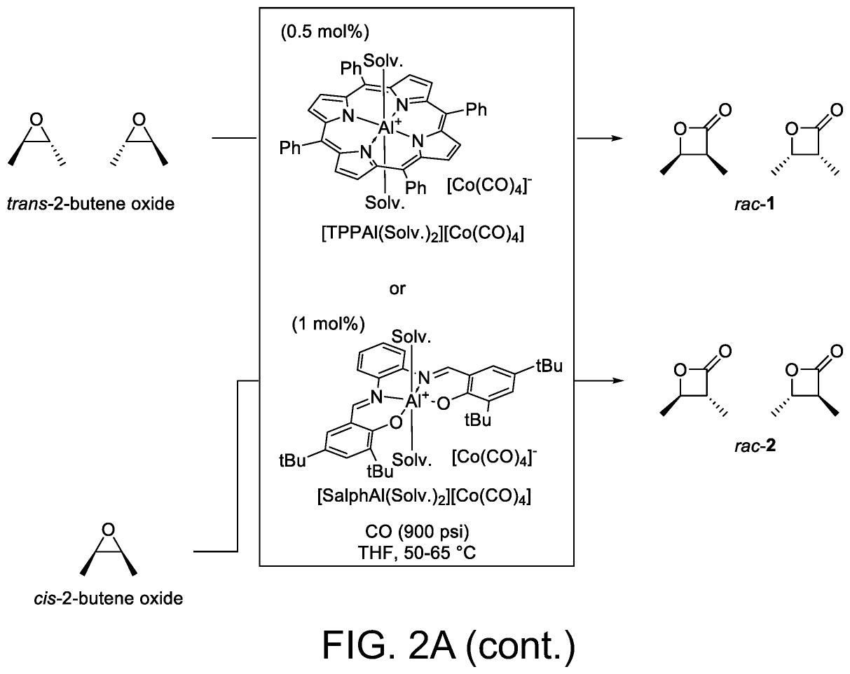

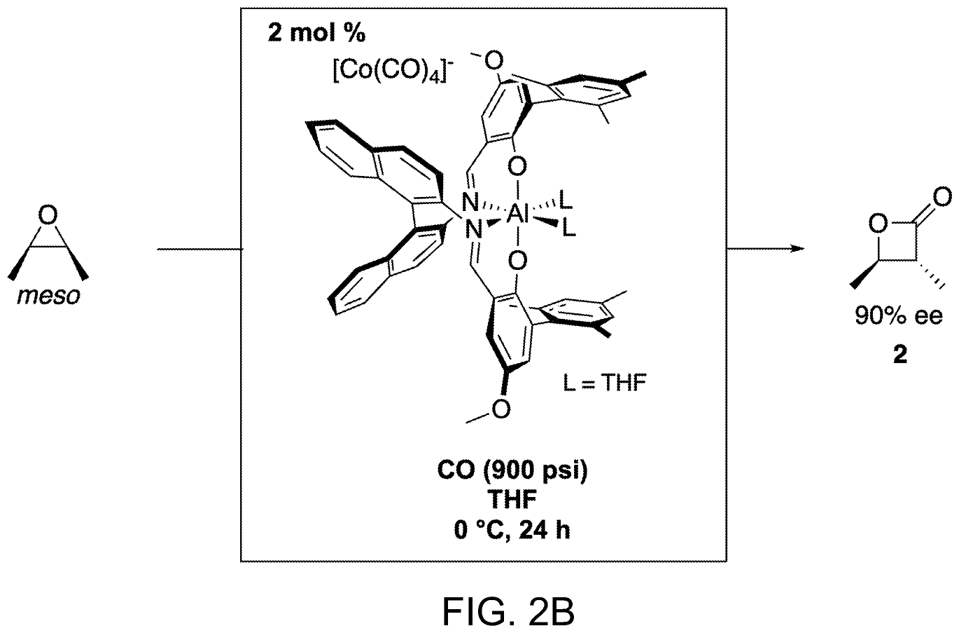

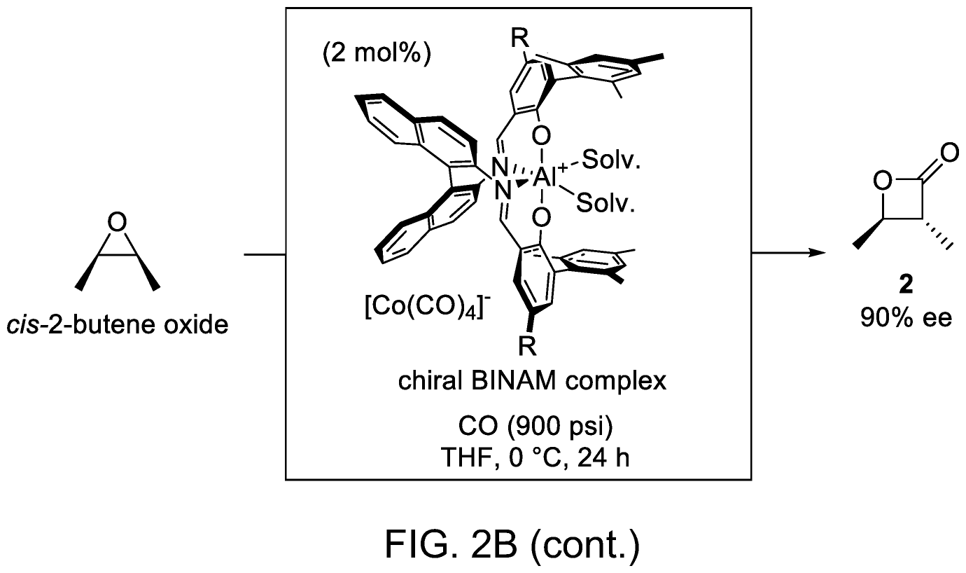

The synthesis of cis- (1) and trans-2,3-dimethyl-β-propiolactone (2) by catalytic carbonylation of trans- and cis-2-buteneoxide, respectively, has been previously reported. 2-buteneoxide is produced by epoxidation of 2-butene, which is a byproduct of the oil refining process. Racemic cis- (rac-1) and trans-2,3-dimethyl-β-propiolactone (rac-2) were obtained in high yield using [salphAl(THF)₂][Co(CO)₄] or [TPPAl(THF)₂][Co(CO)₄] (FIG. 2A). Enantioenriched 2 was prepared using chiral BINAM carbonylation catalysts (FIG. 2B).

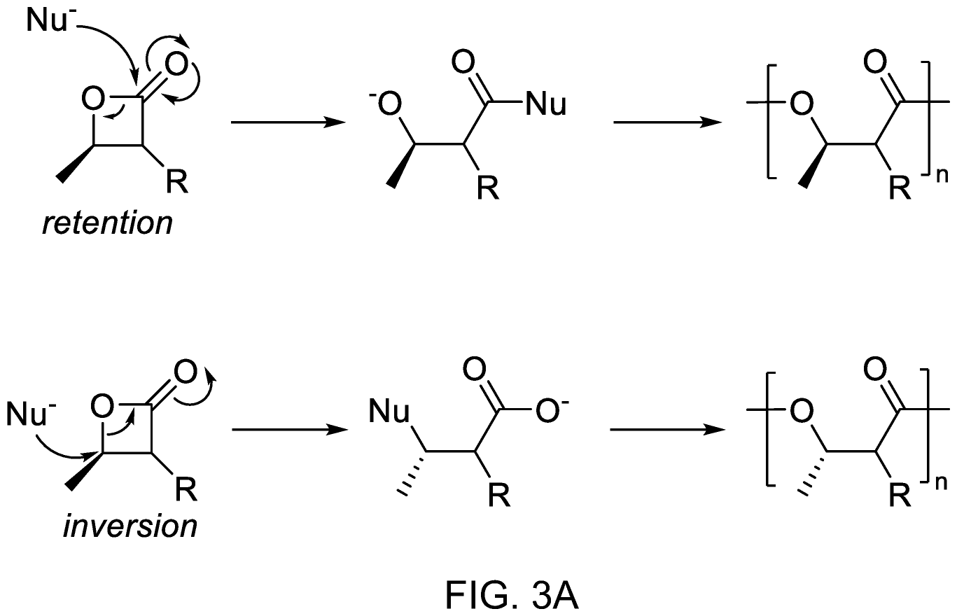

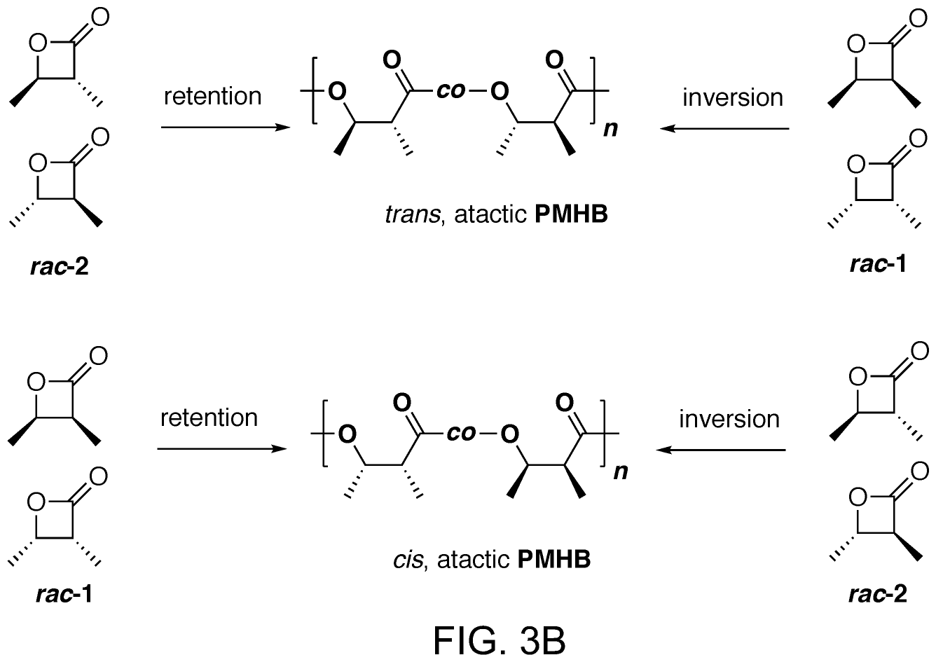

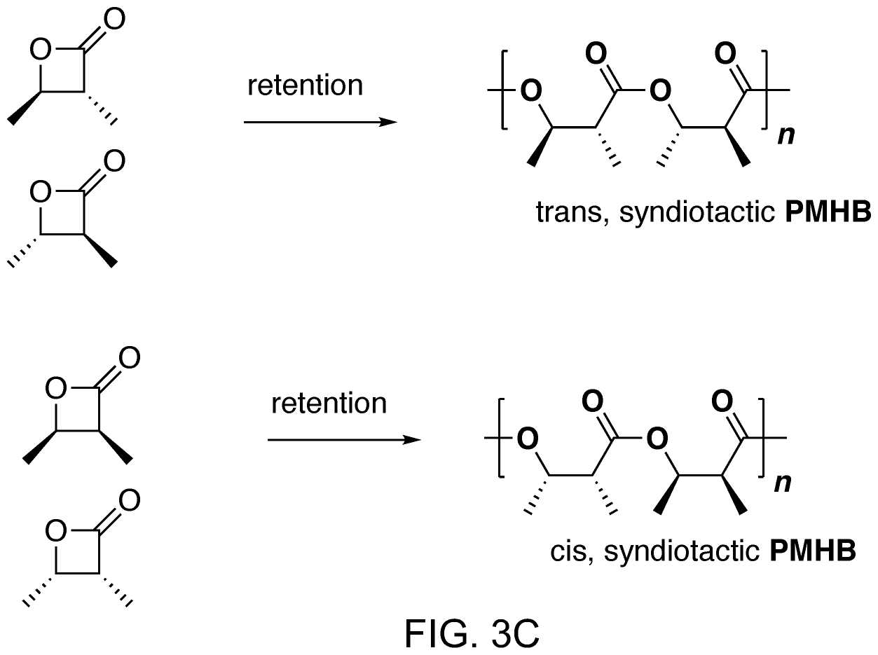

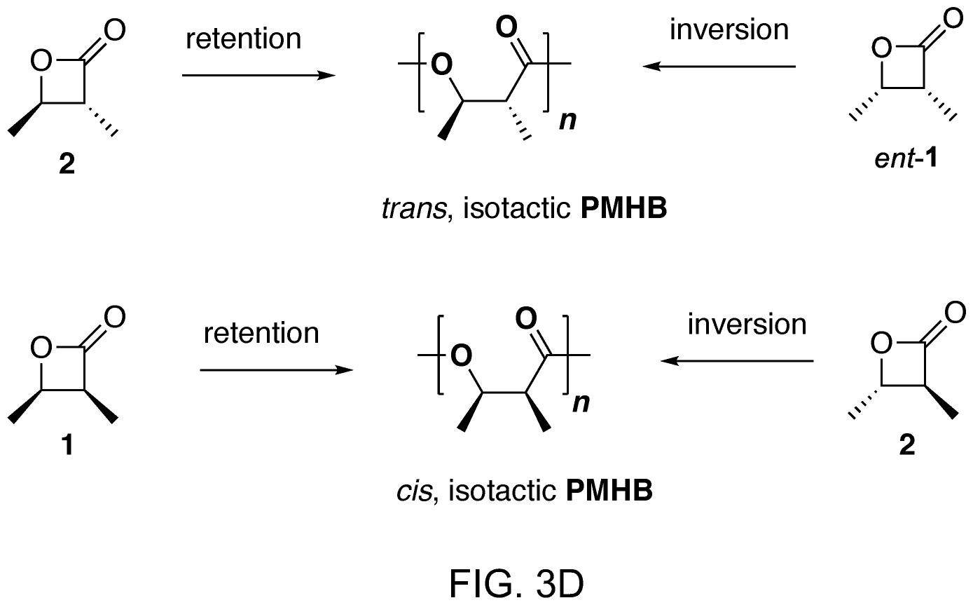

The presence of two adjacent stereocenters in the repeating unit offers the possibility of a large number of possible microstructures (FIG. 3A-3D). In addition, ring-opening polymerization of lactones may occur with inversion (reference) or retention (reference) of stereochemistry. In theory, polymers with the same microstructure may be obtained from either 1 or 2 by varying the type of catalyst used for the polymerization (FIG. 3A-3D).

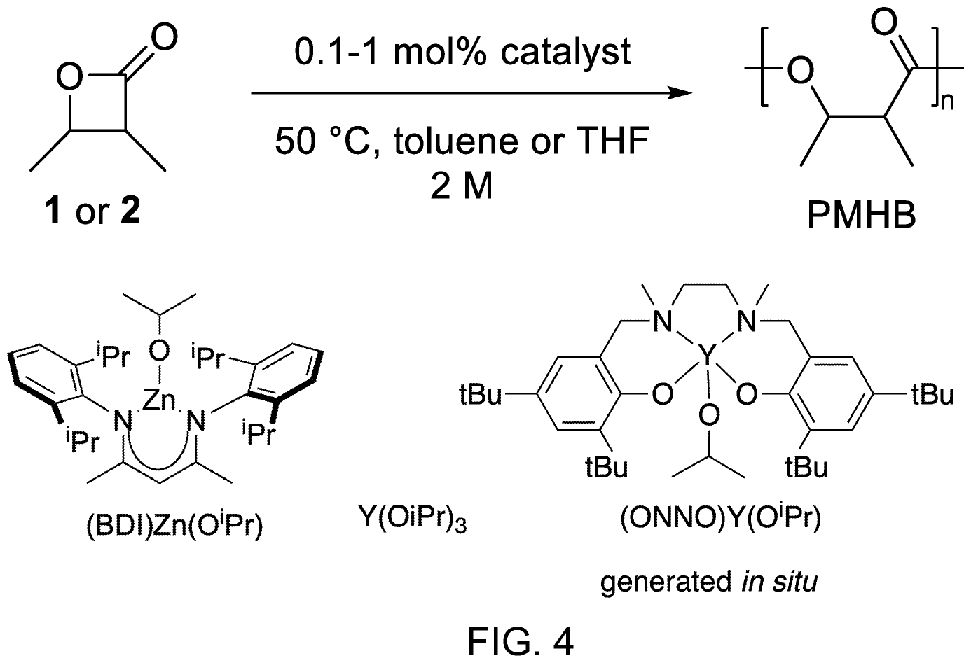

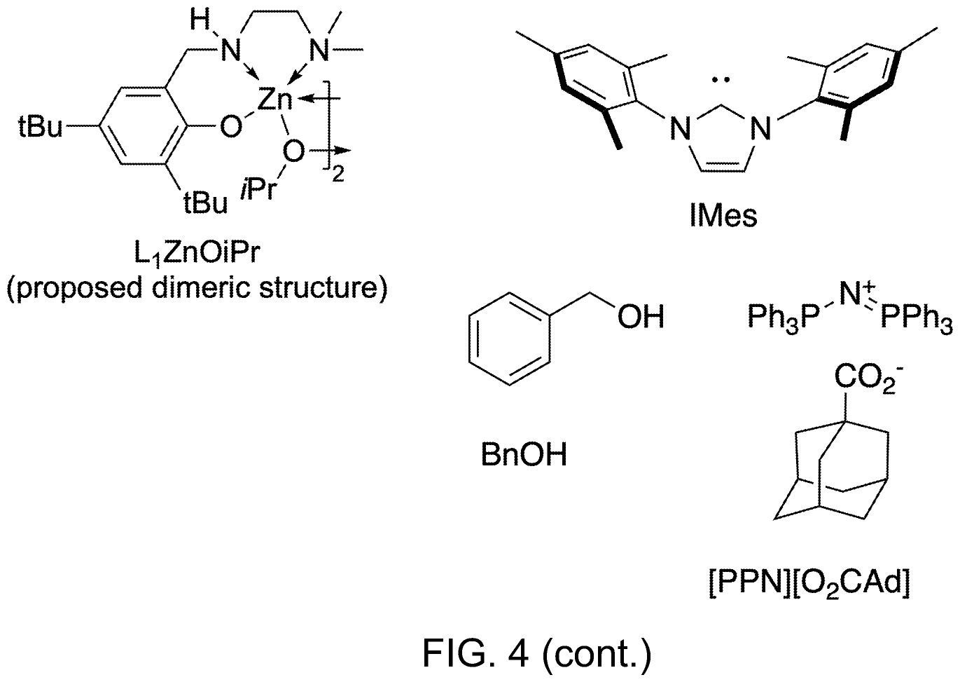

Polymerization Studies of 1 and 2. Metal alkoxide catalysts have been reported to polymerize β-butyrolactone via nucleophilic attack of alkoxide at the carbonyl carbon, resulting in retention of the lactone stereochemistry. The subsequent chain propagation step may proceed with no stereochemical control, yielding atactic polymer. Rare earth alkoxide complexes have also been reported to catalyze the syndiospecific polymerization of β-butyrolactone or the isospecific polymerization of buyrolactone diolides or lactide. Carboxylate salts such as PPNOAc (PPN=bis(triphenylphosphine)iminium) have been reported to ring open lactones with inversion of stereochemistry, and both modes of ring opening have been reported for N-heterocyclic carbenes (NHC) organocatalysts. The polymerization of rac-1 and rac-2 was investigated using a variety of ring-opening polymerization catalysts (Table 1 and FIG. 4). The polymers were characterized by ¹H and ¹³C NMR, differential scanning calorimetry (DSC) and gel-permeation chromatography (GPC).

| | | | Eq. | time | Conv. | | | | stereo- | |
|---|---|---|---|---|---|---|---|---|---|---|
| Lactone | catalyst | solvent | lactone | (h) | NMR, %) | Mn (kDa) | Mw/Mn | microstructure | regularity | Tₘ |
| rac-2 | (BDI)Zn(OⁱPr) | THF | 100 | 24 | 93 | 6.86 | 1.09 | trans atactic | | |
| rac-2 | (BDI)Zn(OⁱPr) | toluene | 100 | 24 | >99 | 11.2 | 1.07 | trans atactic | | |
| rac-2 | (BDI)Zn(OⁱPr) | toluene | 500 | 24 | 92 | 42.3 | 1.09 | trans atactic | | |
| 2 (90% ee) | (BDI)Zn(OⁱPr) | THF | 400 | 24 | 80 | 21.8 | 1.09 | trans isotactic | Pₘ = 0.90 | 168 |
| rac-2 | Y(OⁱPr)3 | toluene | 100 | 24 | >99 | 3.4 | 1.22 | trans atactic | | |
| rac-2 | PPNO₂CAd | THF | 100 | 24 | >99 | 9.1 | 1.22 | cis atactic | | |
| rac-2 | PPNO₂CAd | toluene | 100 | 24 | >99 | 12.2 | 1.1 | cis atactic | | |
| rac-2 | PPNO₂CAd | toluene | 200 | 24 | >99 | v. insoluble | n.d. | cis atactic | | |
| rac-2 | IMes | THF | 100 | 24 | 91 | 12.3 | 1.31 | cis atactic | | |
| rac-2 | IMes/BnOH | THF | 100 | 24 | 90 | 12.9 | 1.22 | cis atactic | | |
| rac-2 | Y(OⁱPr)3 | toluene | 100 | 24 | >99 | 3.4 | 1.22 | trans atactic | | |
| rac-2 | (ONNO)Y(OⁱPr) | THF | 100 | 24 | >99 | 21.3 | 1.46 | trans syndiotactic | | |
| rac-2 | (ONNO)Y(OⁱPr) | toluene | 200 | 24 | >99 | 27.8 | 1.13 | trans syndiotactic | | |
| rac-2 | (ONNO)Y(OⁱPr) | toluene | 400 | 24 | >99 | 31.6 | 1.11 | trans syndiotactic | | |
| rac-2 | (ONNO)Y(OⁱPr) | toluene | 1000 | 24 | >99 | 107 | 1.4 | trans syndiotactic | Pᵣ = 0.83 | |
| rac-1 | (BDI)Zn(OⁱPr) | THF | 100 | 3 | >99 | 9.7 | 1.06 | cis syndioenriched | Pᵣ = 0.77 | 189 |
| rac-1 | PPNO₂CAd | THF | 100 | 2 | >99 | 8.9 | 1.11 | trans atactic | | amorphous |
| rac-1 | Y(OⁱPr)₃ | THF | 100 | 24 | >99 | 5.7 | 1.2 | cis syndioenriched | Pᵣ = 0.77 | 188 |
| rac-1 | (ONNO)Y(OⁱPr) | THF | 100 | 24 | >99 | v. insoluble | n.d. | cis syndiotactic | Pᵣ = 0.95 | |
| rac-1 | (ONNO)Y(OⁱPr) | DCM | 500 | 1.0 | >99 | 122 | 1.03 | cis syndiotactic | Pᵣ = 0.95 | 206 |

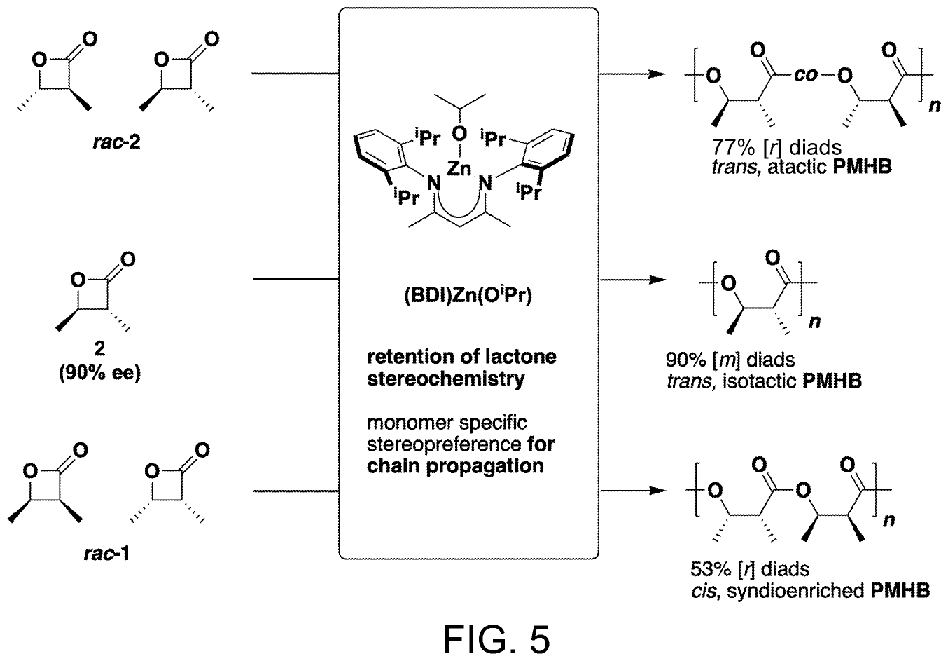

The polymerization of rac-1 and rac-2 by zinc (β-diimine (BDI)) complex (BDI)Zn(OⁱPr) was investigated (Table 1, FIG. 5). The polymerization of the trans-lactone, rac-2, by (BDI)Zn(OⁱPr) yields a sticky oil whose NMR is consistent with the formation of trans, atactic PMHB. In contrast, the polymerization of enantioenriched 2 (90% ee) by (BDI)Zn(OⁱPr) yields a semi-crystalline polymer with Tₘ=170° C. ¹³C analysis of the carbonyl carbon (172 ppm) shows Pₘ value of 0.90 (90% [m] diads) (Table 1, FIG. 5) These results are consistent with retention of stereochemistry during the lactone ring-opening step and no stereocontrol of the propagation step. The polymerization of rac-1 by (BDI)Zn(OⁱPr) yields a white powder with Tₘ=188° C. ¹H and ¹³C NMR of the material are different than those observed for trans, atactic PMHB, and are consistent with slightly syndioenriched cis PHMB Pᵣ=0.77 (77% [r] diads) (Table 1, FIG. 5). Similar results were observed for Y(OⁱPr)₃, with trans, atactic PMHB being obtained from rac-2 and slightly syndioenriched cis PMHB Pᵣ=0.77 (77% [r] diads) being obtained from rac-1 (Table 1).

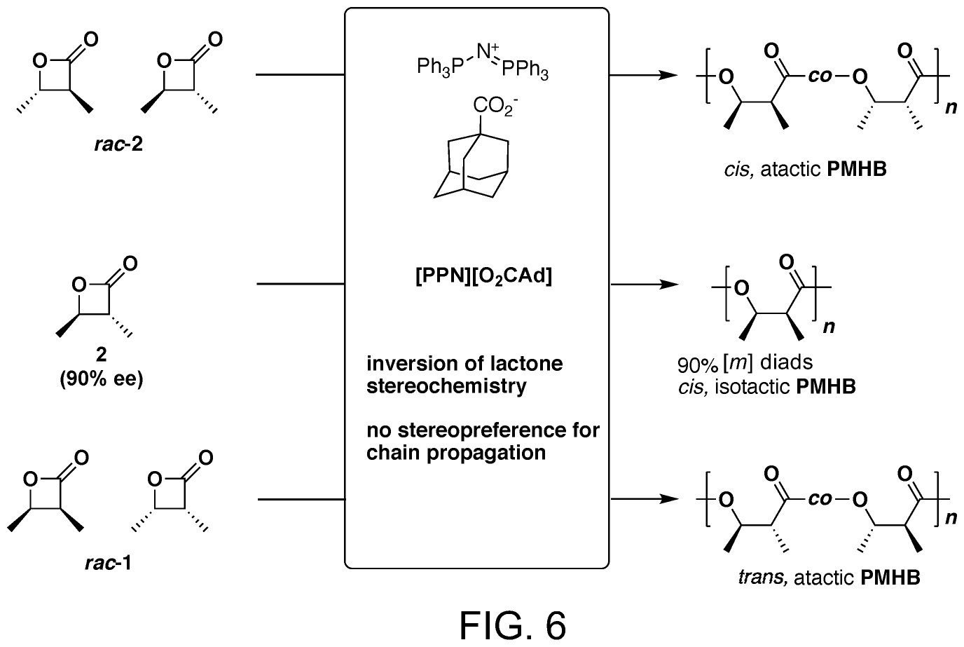

The polymerization of rac-1 and rac-2 by bis(triphenylphosphine)iminium complex [PPN][O₂CAd] (Ad: adamantyl) was investigated (see also FIG. 6). The polymerization of rac-2 yielded a white powder whose NMR data were different than that of trans, atactic PMHB. The identity of product was assigned to be cis, atactic PMHB. Similarly, the polymerization of the cis lactone, rac-1, yielded a sticky oil whose NMR data were consistent with that of trans, atactic PMHB. When isoenriched 2 (90% ee) was polymerized by [PPN][O₂CAd], a highly crystalline white powder was obtained which was very insoluble in THF. NMR data were consistent with the formation of cis, isotactic PMHB. These results are consistent with ring opening with inversion of lactone stereochemistry and no stereocontrol during the chain propagation step. The polymerization of rac-1, rac-2 and enantioenriched 2 by the N-heterocyclic carbene (IMes) yielded products consistent with ring-opening with inversion of the lactone stereochemistry and no stereochemical control of the chain propagation step. Similar results were obtained when a combination of IMes and benzyl alcohol cocatalyst was used.

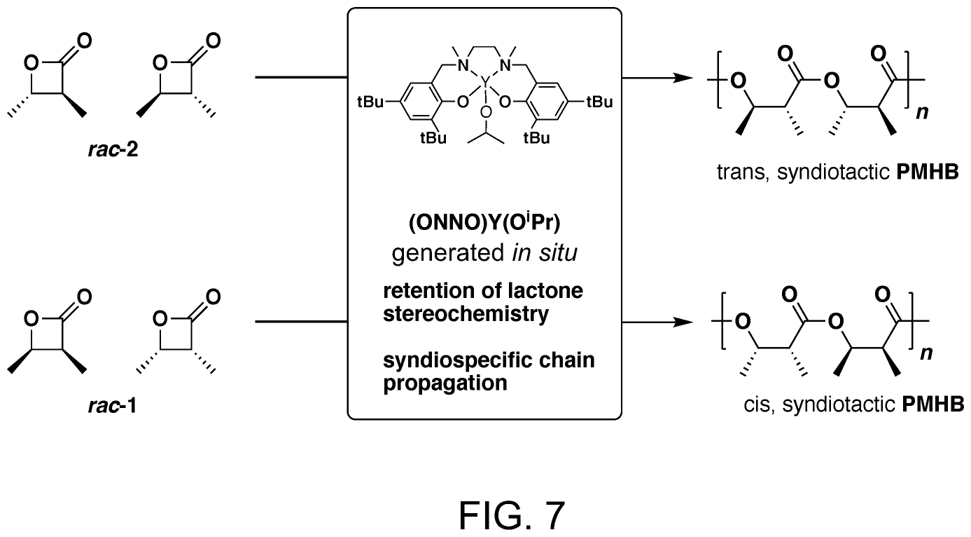

The polymerization of rac-1 and rac-2 by yttrium bis(amino-methylphenoxide) complex (ONNO)Y(OⁱPr) was investigated (see also FIG. 7). The (ONNO)Y(OⁱPr) complex has been reported to polymerize β-butyrolactone with >90% syndiospecificity. The polymerization of rac-2 by (ONNO)Y(OⁱPr) yielded a semicrystalline polymer which was soluble in THF and toluene. NMR data were consistent with the formation of trans, syndiotactic PMHB (Pᵣ=0.80-0.85). In contrast, the polymerization of rac-1 by (ONNO)Y(OⁱPr) yielded a highly insoluble white polymer. Comparison with NMR data for cis, atactic and cis, isotactic PHMD revealed that the product was highly syndioenriched cis PMHB (Pᵣ=0.95). These results are consistent with ring opening with retention of lactone stereochemistry and a syndiospecific chain propagation step.

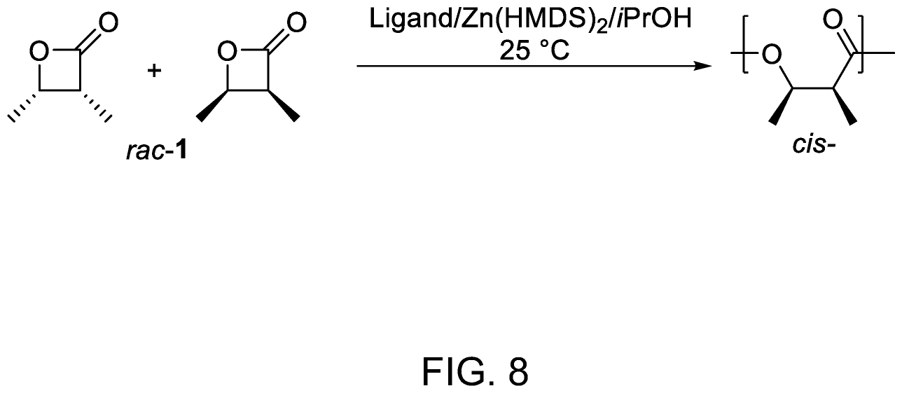

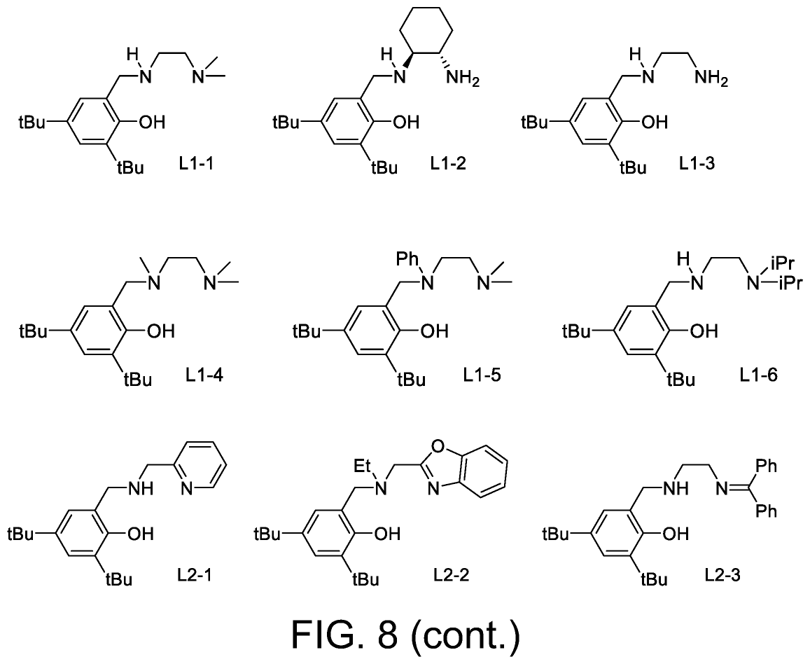

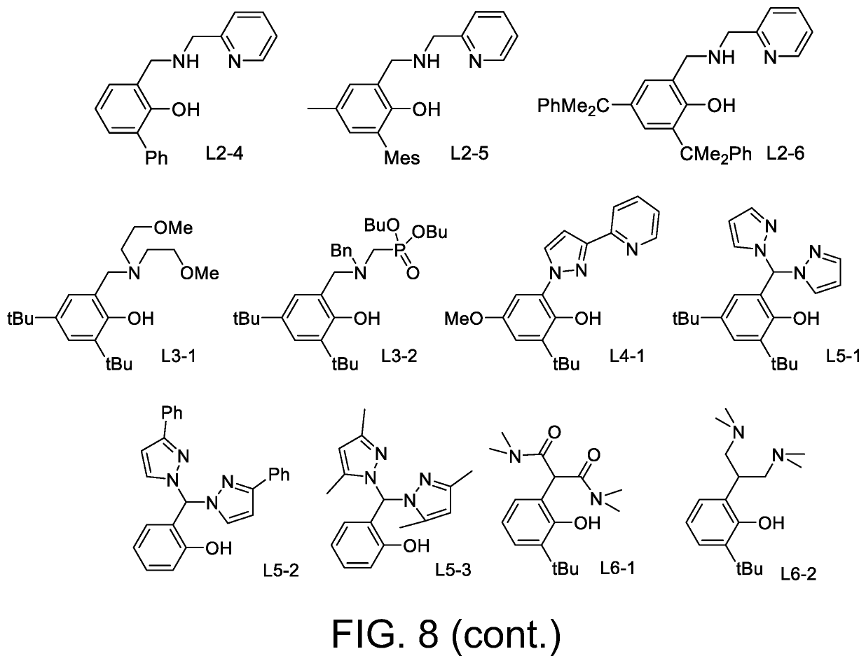

The remarkable activity and syndiotacticty of yttrium complex (ONNO)Y(OⁱPr) towards polymerization of both 1 and 2 yields polymers with promising properties, but the relatively high cost of the yttrium metal might limit its use in large scale industrial production. Given the success in (BDI)Zn(OⁱPr) catalyzed polymerization of both 1 and 2, more economical Zn-based catalysts that can achieve comparable activity and selectivity of (ONNO)Y(OⁱPr) system were pursued. Various phenol-containing ligands were designed and synthesized and their activity (FIG. 8) and selectivity with in situ formed phenoxylzinc isopropoxide [ONN]Zn(OⁱPr) (Ligand/Zn(HMDS)₂/iPᵣOH) systems for the polymerization of rac-1 was tested. The results are summarized in Table 2 and the polymers obtained were characterized by NMR, DSC and GPC. In general, almost all [ONN]Zn(OⁱPr) catalysts gave syndioenriched cis-polymers from rac-1 with Pᵣ=0.6-0.7, and the [rr] triad ratio matched Bernoullian model, indicating chain-end control with modest syndioselectivity regardless of the chirality of the Zn-complexes. Interestingly, L5-2 led to the formation of iso-enriched cis-PMHB from rac-1. Notably, most polymers obtained from the more active [ONN]Zn(OⁱPr) systems (above 50% conversion) had relatively high molecular weights as well as high Tₘ (186-190° C.).

| | | | | | conv. | | | | | |
|---|---|---|---|---|---|---|---|---|---|---|
| | | | eq | time | (NMR, | | | | stereo- | |
| Lactone | ligand | solvent | lactone^{a} | (h) | %) | Mn (kDa) | Mw/Mn | microstructure | regularity | Tₘ |
| rac-1 | L1-1 | DCM | 1000 | 3.5 | >99 | 80.5 | 1.03 | cis syndioenriched | Pᵣ = 0.63 | 187 |
| rac-1 | L1-1 | DCM | 2000 | 16 | >99 | 116.7 | 1.02 | cis syndioenriched | Pᵣ = 0.63 | |
| rac-1 | L1-2 | DCM | 2000 | 24 | ND | 76.7 | 1.03 | cis syndioenriched | Pᵣ = 0.65 | 188 |
| rac-1 | L1-3 | DCM | 2000 | 24 | 33 | 35.6 | 1.02 | cis syndioenriched | Pᵣ = 0.60 | 186 |
| rac-1 | L1-4 | DCM | 1400^{b} | 6 | >99 | 126.3 | 1.02 | cis syndioenriched | Pᵣ = 0.67 | |
| rac-1 | L1-5 | DCM | 1400^{b} | 24 | 55 | 49.5 | 1.03 | cis syndioenriched | Pᵣ = 0.70 | |
| rac-1 | L1-6 | DCM | 1400^{b} | 24 | 22 | 20.6 | 1.02 | ND | ND | |
| rac-1 | L2-1 | DCM | 2000 | 24 | >99 | 91.9 | 1.02 | cis syndioenriched | Pᵣ = 0.68 | 190 |
| rac-1 | L2-2 | DCM | 2000 | 24 | 95 | 64.5 | 1.02 | cis syndioenriched | Pr = 0.63 | 188 |
| rac-1 | L2-3 | DCM | 2000 | 24 | 41 | 34.8 | 1.02 | cis syndioenriched | Pᵣ = 0.62 | 186 |
| rac-1 | L2-4 | DCM | 1400^{b} | 24 | 23 | 16.0 | 1.07 | ND | ND | |
| rac-1 | L2-5 | DCM | 2000 | 24 | 47 | 36.9 | 1.03 | cis syndioenriched | Pᵣ = 0.66 | 189 |
| rac-1 | L2-6 | DCM | 1400^{b} | 7 | 77 | 70.0 | 1.06 | cis syndioenriched | Pᵣ = 0.65 | |
| rac-1 | L3-1 | DCM | 2000 | 24 | 97 | 100.2 | 1.02 | cis syndioenriched | Pᵣ = 0.65 | |
| rac-1 | L3-2 | DCM | 1400^{b} | 24 | >99 | 114.9 | 1.02 | cis syndioenriched | Pᵣ = 0.62 | 186 |
| rac-1 | L4-1 | DCM | 1400^{b} | 24^{c} | ND | 73.1 | 1.06 | cis syndioenriched | Pᵣ = 0.62 | |
| rac-1 | L5-1 | DCM | 1400^{b} | 24 | ND | 86.2 | 1.03 | cis syndioenriched | Pᵣ = 0.68 | |
| rac-1 | | | | | | 112.1; | 1.03; | | | |
| | L5-2 | DCM | 1400^{b} | 120 | 86 | 52.0 | 1.10 | cis isoenriched | Pₘ = 0.85 | 188 |
| rac-1 | L5-3 | DCM | 1400^{b} | 24 | >99 | 101.0 | 1.04 | cis syndioenriched | Pᵣ = 0.75 | 191 |
| rac-1 | L6-1 | DCM | 1400^{b} | 24 | >99 | 118.3 | 1.02 | cis syndioenriched | Pᵣ = 0.64 | |
| rac-1 | L6-2 | DCM | 1400^{b} | 6 | 83 | 109.7 | 1.05 | cis syndioenriched | Pᵣ = 0.69 | 190 |

^{a}monomer concentration = 2M unless otherwise noted;
^{b}monomer concentration = 4M;
^{c}25° C. for 50 h, then 50° C., 24 h.

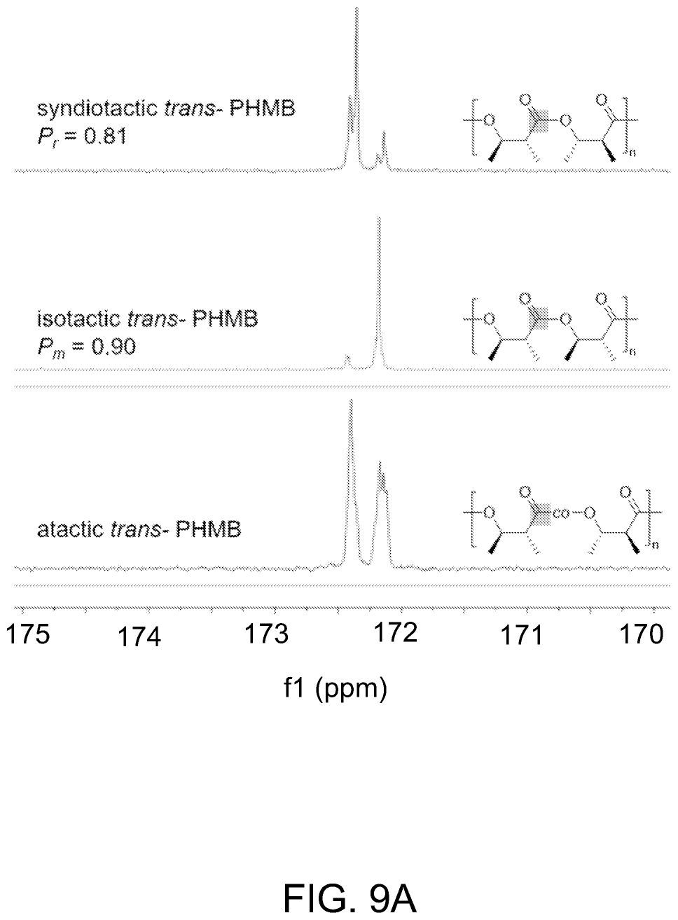

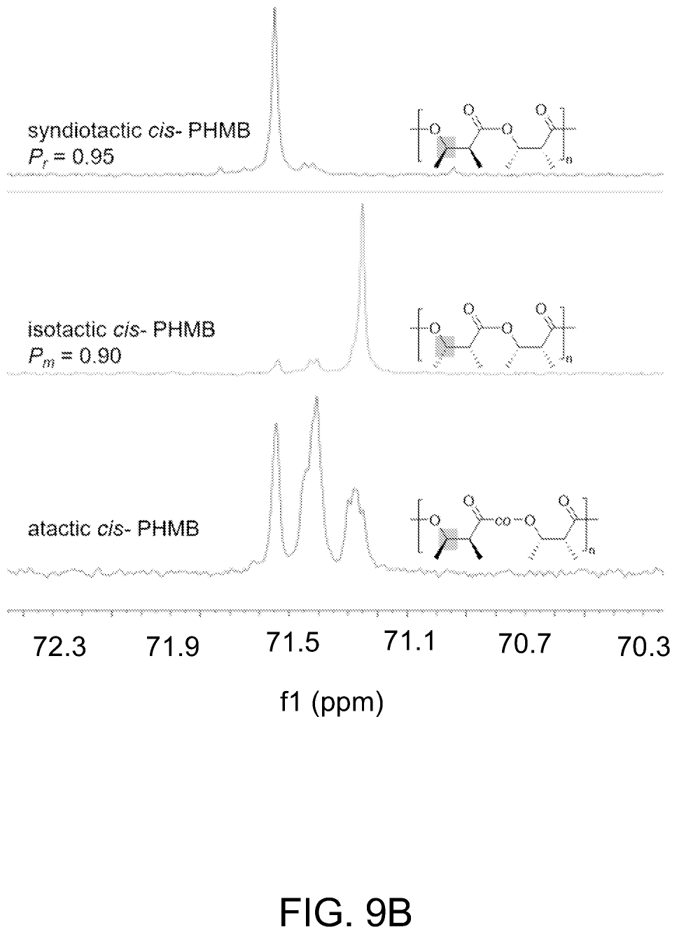

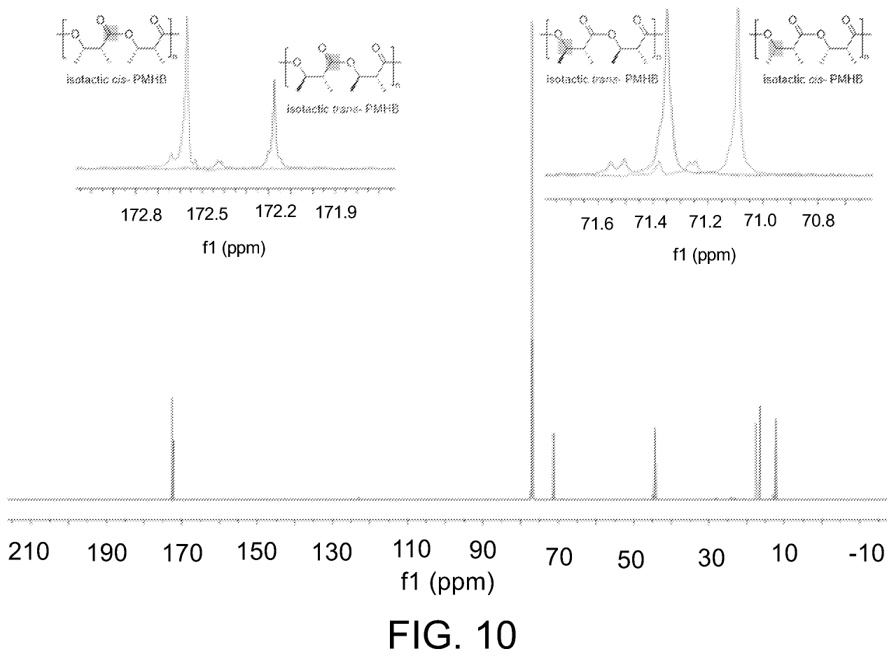

Characterization of PMHB. The various microstructures of PMHB exhibit distinct features in their NMR spectra and DSC analysis. Although the ¹H NMR spectra for cis and trans PMHB show similar splitting patterns, the ¹H and ¹³C chemical shifts are slightly different. Likewise, it is possible to distinguish between syndio-, iso- and atactic PMHB and quantify the relative amounts of stereoregularity using quantitative ¹³C NMR. Selected ¹³C NMR data for the different cis and trans-PMHB microstructures are shown in FIG. 9A-9B and a comparison of the cis and trans isomers is shown in FIG. 10.

DSC and TGA analysis. The PMHB samples were analyzed by DSC and thermal gravimetric analysis (TGA) and the data are summarized in Table 3. In general, high decomposition temperatures (Td, 260-287° C.) were observed, consistent with the hypothesis that adding a methyl substitution at the 2-position increases the thermal stability of the polymers relative to PHB. The glass transition temperature (Tg) for all microstructures of trans-PMHB is ca. 10-20° C. Due to the high crystallinity of the syndiotactic and isotactic trans-PMHB samples, no Tg was observed with the standard heating and cooling protocol. It was necessary to perform flash cooling of the samples immediately prior to running the DSC in order to minimize crystallization. For cis-PMHB, Tg's have not yet been observed.

TABLE 3

| Methyl | | % stereo- | Tm | Tc | Td | Melt | Cryst |
|---|---|---|---|---|---|---|---|
| config. | tacticity | regular | (deg. C.) | (deg. C.) | (deg. C.) | (J/g) | (J/g) |
| trans | atactic | n.a. | amorphous | | 282 | amorphous | |
| trans | isotactic | Pₘ = 0.90 | 168 | 104 | 287 | −49 | 45 |
| trans | syndiotactic | Pᵣ = 0.87 | 159, 171 | 131 | 267 | −32 | 38 |
| cis | atactic | n.a. | 145-175* | 133 | 280 | −37 | 49 |
| cis | isotactic | Pₘ = 0.90 | 180-200* | 166 | | −54 | 45 |
| cis | syndiotactic | Pᵣ = 0.95 | 180-206* | 186 | | −40 | 46 |

*multiple broad melt transitions observed

The crystallinity of all microstructures of cis-PMHB was unexpected. Although isotactic and syndiotactic polymers may exhibit semi-crystallinity and high melting points, this is much less common with atactic polymers, especially those containing multiple stereocenters per repeating unit. Although rare, tacticity-independent crystallization has been observed in a few instances. For instance, atactic poly(vinylene-cis-1,3-cyclopentylene) [fully hydrogenated polynorbornene, (HPN)] is semi-crystalline.

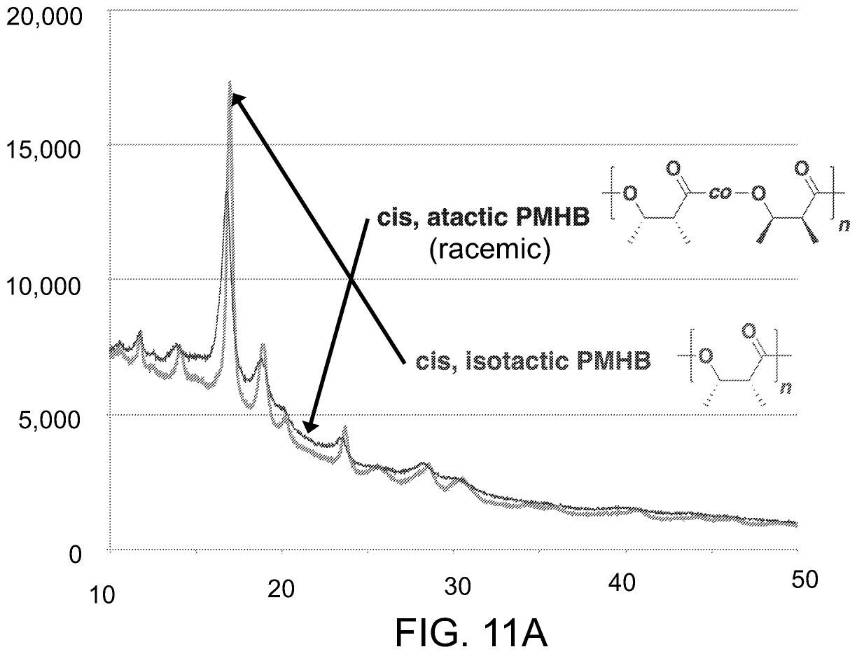

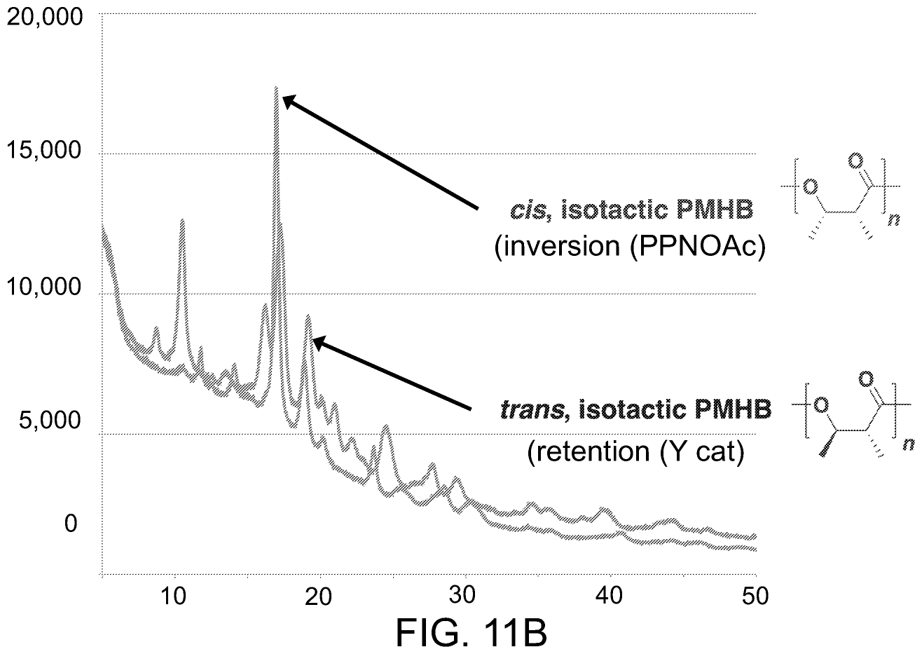

The HPN crystallinity is attributed to its tendency to crystallize into the same unit cell even in the presence of a high degree of local structural disorder arising from the presence of multiple stereoisomers in the repeating unit. An atactic polythioester containing a 1,3-cyclopentyl linkage in the main chain has been reported that also exhibits tacticity-independent crystallization. A similar phenomenon may be occurring in cis PMHB. Samples of cis, atactic and cis, isotactic PMHB were analyzed by powder X-ray diffraction (PXRD) (FIG. 11A). A similar powder pattern was obtained, suggesting that the unit cell may be the same in the two cis microstructures. In contrast, the PXRD pattern for trans, isotactic PMHB was distinct from the cis polymers (FIG. 11B).

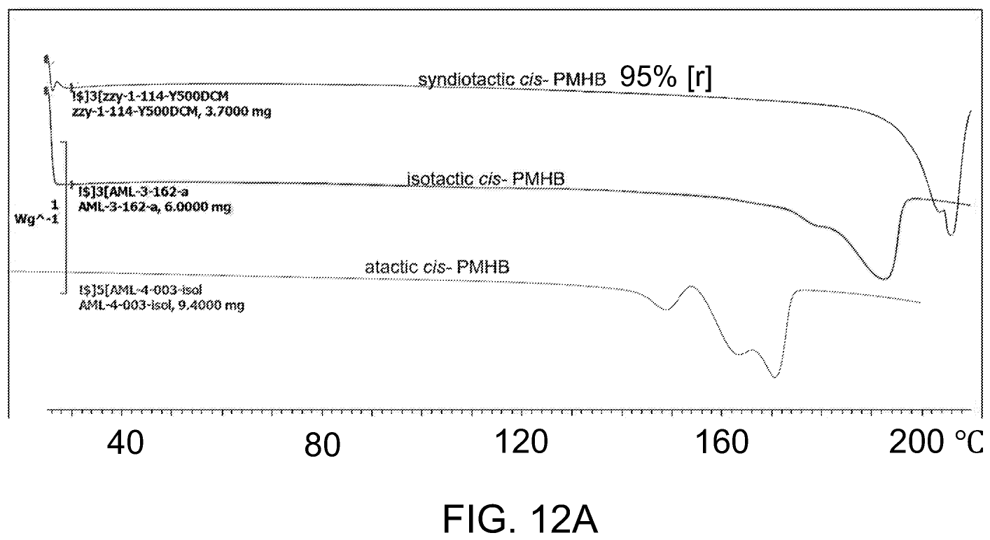

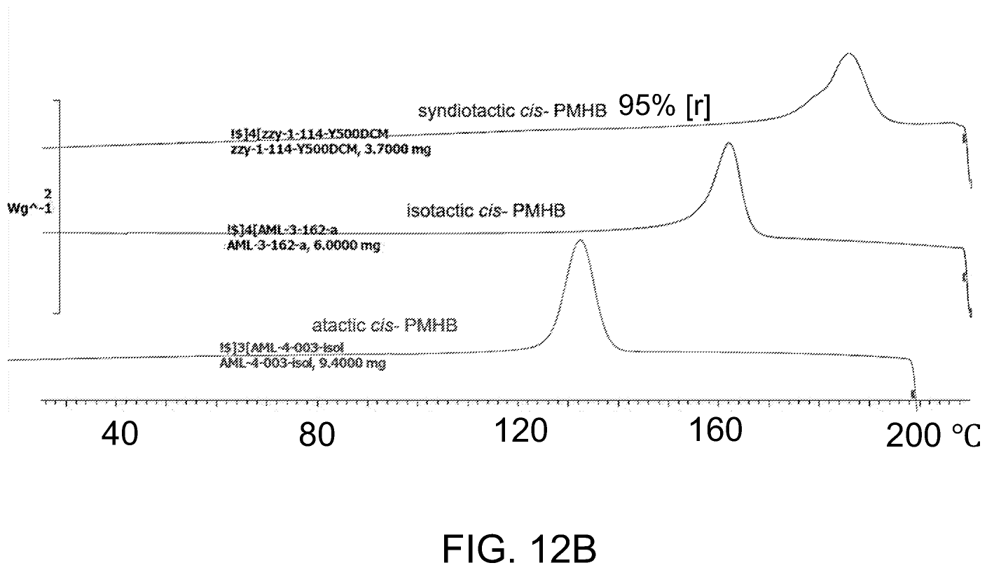

The heat of crystallization for the various microstructures of PMHB is shown in Table 3. Similar heats of crystallization are observed for trans-isotactic and the three cis microstructures. Although heats of fusion and crystallization of stereoregular semi-crystalline polymers is usually proportional to their overall stereoregularity, the heat of crystallization for cis, atactic PMHB is very similar to that of the cis, syndiotactic and cis, isotactic materials. PMHB shows complicated melting behavior which is sensitive to the thermal history of the sample as well as the overall microstructure. DSC traces for the heating (second melt) (FIG. 12A) and the cooling (crystallization) (FIG. 12B) transitions of the various microstructures of cis PMHB are shown.

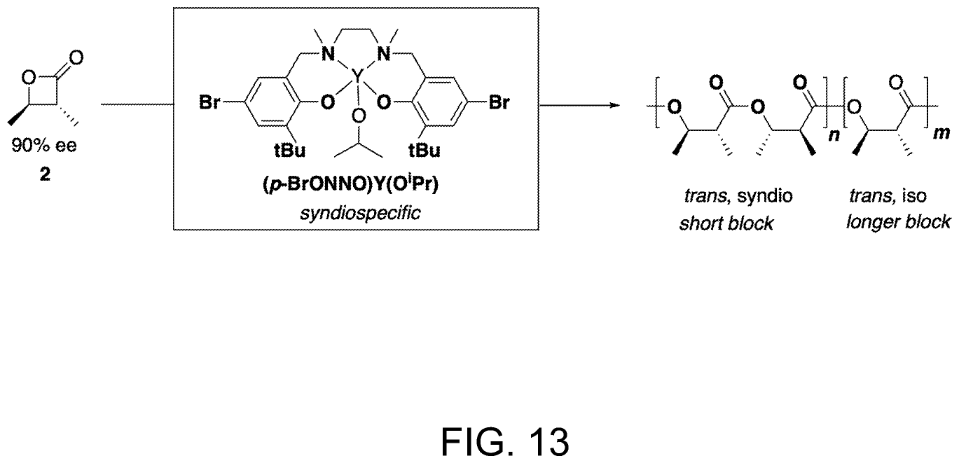

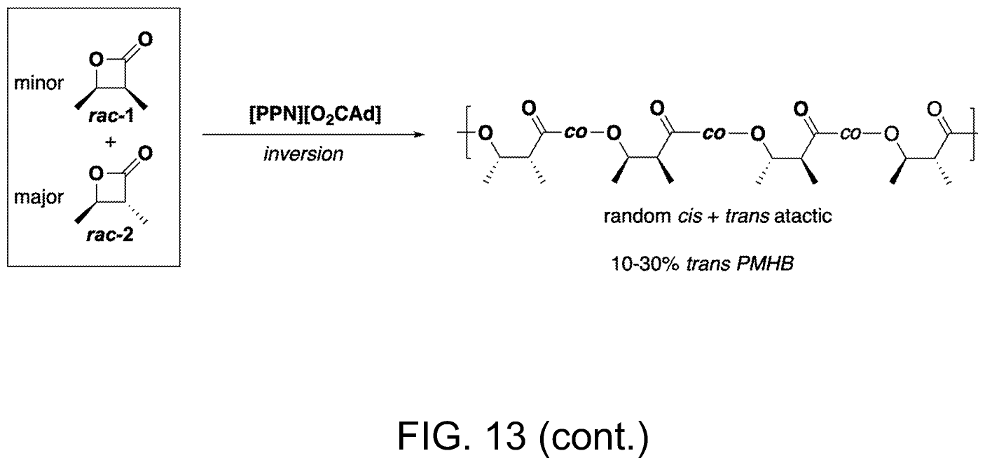

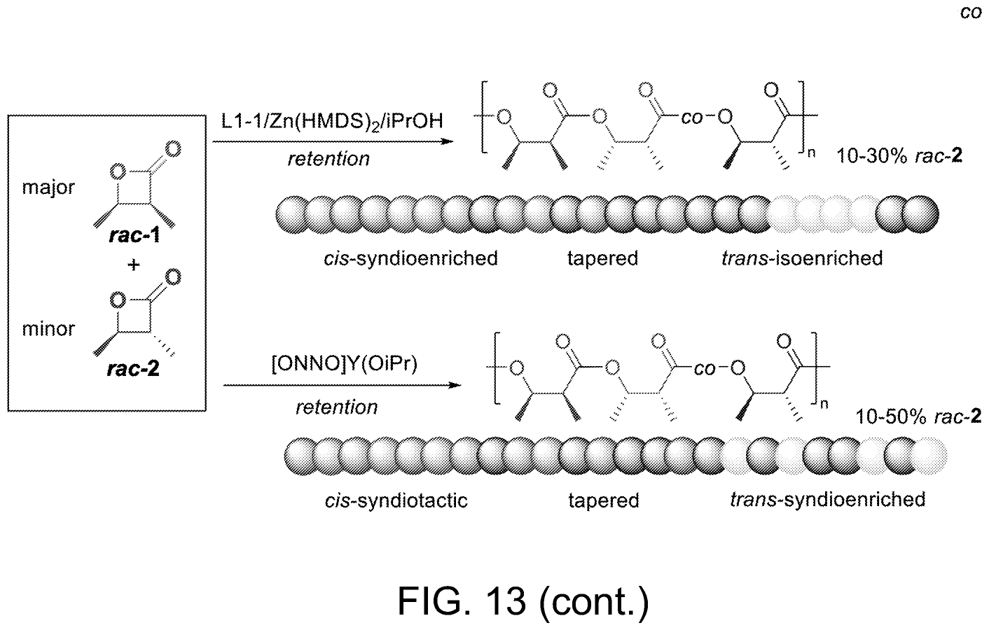

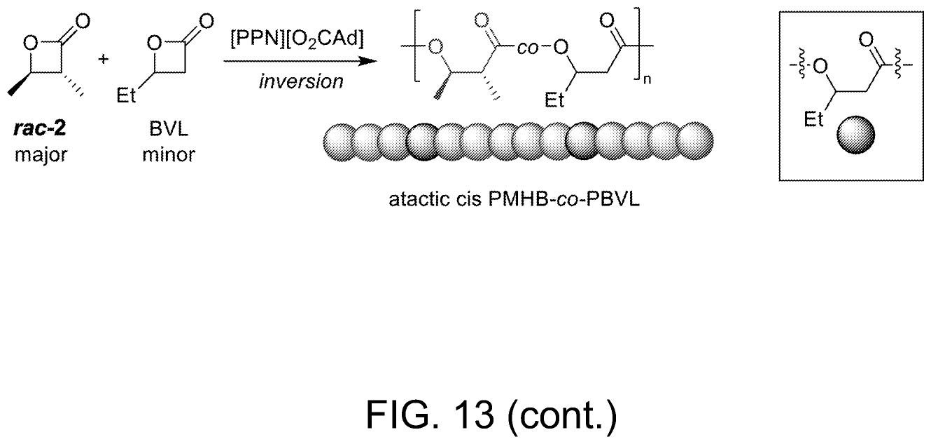

Copolymerization of cis and trans 2,3-dimethyl-β-propiolactone and preparation of other PMHB copolymers. The wide range of observed PMHB microstructures and the ability to access a single isomer (cis vs trans) from either diastereomer of 2,3-dimethyl-β-propiolactone potentially allows the preparation of higher order PMHB architectures such as block, tapered and random copolymers of cis and trans and/or iso-/syndio/atactic PMHB by tuning the composition of the monomer feed or varying the catalyst (FIG. 13). For instance, the polymerization of enantioenriched 2 by the syndiospecific catalyst [p-Br-ONNO]Y(OⁱPr) affords trans, isotactic PMHB with Tₘ=200° C., a 30° C. increase over that produced when 2 is polymerized by (BDI)Zn(OⁱPr). ¹³C NMR analysis of the polymer shows 90% [m] diads, consistent with the ee of the monomer feed, but the Tₘ suggests that the polymer has higher overall isotacticity. Based on the highly syndiospecific polymerization observed with the yttrium phenoxide catalyst, it is proposed that a short block of trans, syndio PMHB is produced at the beginning of the polymerization, depleting the feed of the minor enantiomer, followed by a longer block of trans, iso PMHB with higher tacticity. ('is and trans-PMHB copolymers may also be prepared by using a mixture of the cis and trans lactones.

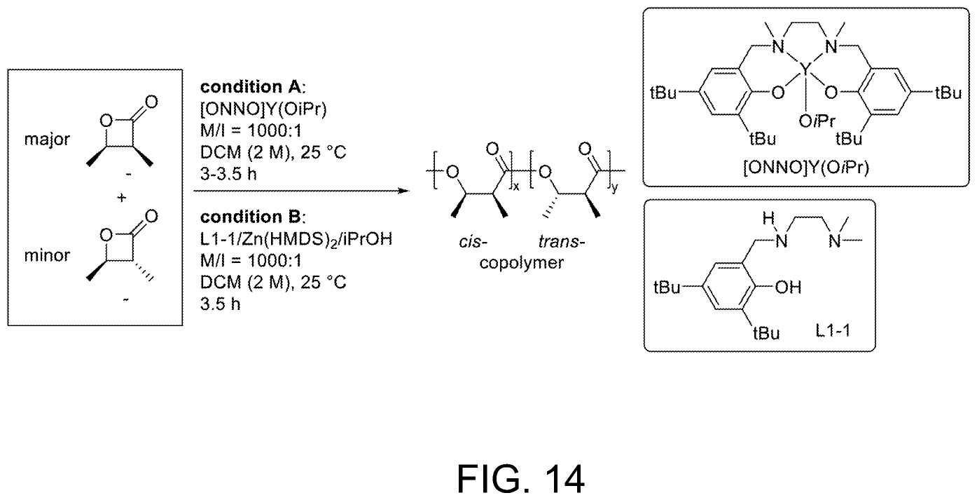

Mixtures of rac-1 and rac-2 were polymerized by [ONNO]Y(OⁱPr) or [PPN][O₂CAd] or [ONN]Zn(OⁱPr) to yield PMHB with mixtures of cis and trans repeat units. A dramatic increase in the solubility and decrease in the Tₘ were observed as the loading of the trans linkages increased in the atactic cis-co-trans copolymer produced by [PPN][O₂CAd]. When the experiment was performed using the syndiospecific catalyst [ONNO)Y(OⁱPr) (See FIG. 14 and Table 4, condition A), the syndioenriched cis-trans polymers showed a less dramatic decrease in Tₘ which was attributed to the higher overall stereoregularity of the polymer. On the other hand, with in situ generated [ONN]Zn(OⁱPr) catalytic system (See Table 4, condition B), high molecular weight copolymer could also be obtained with 10 to 30% ratio of 2 incorporated and lower stereoregularity compared to the [ONNO]Y(OⁱPr) system. Interestingly, the [ONN]Zn(OⁱPr) system led to the iso-enriched trans-copolymer incorporation, as verified by the homo-polymerization of rac-2 with the same catalyst. As expected, a larger drop of Tₘ and T_{c} was observed and no crystallization was observed with 70% cis-/30% trans-PMHB copolymer. In addition, kinetic studies of the homopolymerizations showed that rac-1 is polymerized much faster than rac-2, leading to tapered block copolymers. Other lactones may also be used as comonomers. The addition of up to 20 mol % β-butyrolactone or β-valerolactone monomer to a polymerization of rac-2 by [PPN][O₂CAd] yielded the cis, atactic copolymers with lower Tₘ's and improved solubility.

TABLE 4

| condition | ratio 1/2 | 1 conv. (%) | 2 conv. (%) | Mn (kDa) | Mw/Mn | Tₘ (° C.) | T_{c} (° C.) |
|---|---|---|---|---|---|---|---|
| A | 90/10 | >99 | >99 | 268.9 | 1.16 | 194 | 162 |
| | 80/20 | >99 | >99 | 240.4 | 1.22 | 187 | 146 |
| | 70/30 | >99 | 93 | 264.4 | 1.23 | 177 | 128 |
| | 60/40 | >99 | 85 | 108.6 | 1.08 | 159 | 98 |
| | 50/50 | >99 | 80 | 235.5 | 1.28 | 145 | N/A |
| B | 90/10 | >99 | 93 | 64.8 | 1.07 | 168 | 114 |
| | 80/20 | >99 | 95 | 66.0 | 1.02 | 162 | 99 |
| | 70/30 | >99 | 97 | 57.4 | 1.03 | 130 | N/A |

Preparation of high molecular weight samples. Living polymerization behavior was observed for all catalysts in Table 1 and Table 2. Polymerizations at lower catalyst loadings (0.05-0.5 mol % were performed using [ONNO]Y(OⁱPr), [PPN][O₂CAd] and (BDI)Zn(OⁱPr)This permitted the preparation of high molecular weight samples of trans, syndiotactic and trans, atactic PMHB. Molecular weights above 40,000 were observed as well as narrow dispersities. (Ð<1.3) [ONNO]Y(OⁱPr) and various [ONN]Zn(OⁱPr) catalysts were also employed in the polymerization of rac-1 to prepare high molecular weight samples of cis, syndiotactic and cis, syndioenriched PMHB with >100,000 Da molecular weight and extremely narrow dispersities in the case of [ONN]Zn(OⁱPr) series (in most cases Ð<1.05). The [ONNO]Y(OⁱPr)-catalyzed polymerizations were highly sensitive to chain transfer in the presence of protic impurities, so it is necessary to use extremely pure lactone monomers to achieve high molecular weights.

Tensile properties of PMHB. R-PHB is extremely brittle and requires the use of comonomers to improve its physical properties. In addition, the high Tₘ and low T_{d} result in a narrow processing window and limit its potential applications. The high crystallinity of many microstructures of PMHB and ability to tune the thermal properties by changing the microstructure, architecture or composition are very advantageous and preliminary results suggest that PMHB has high inherent tensile strength.

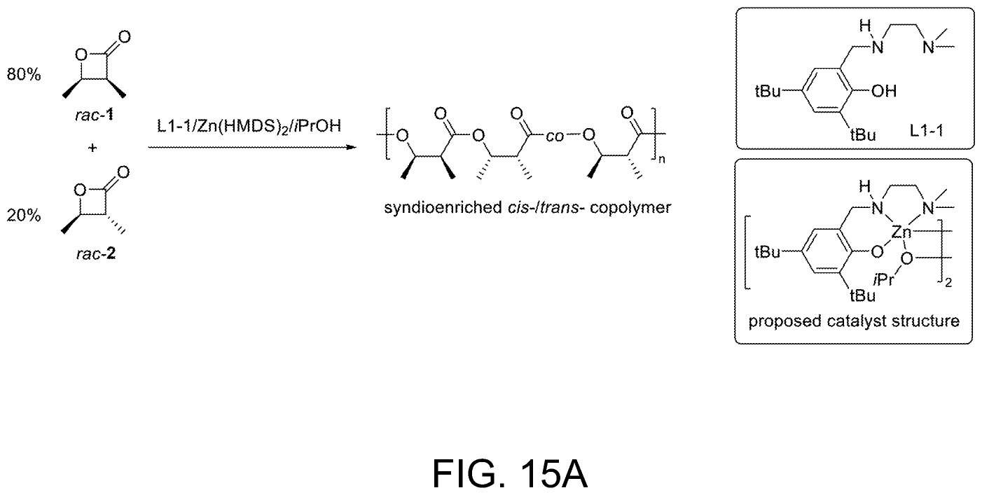

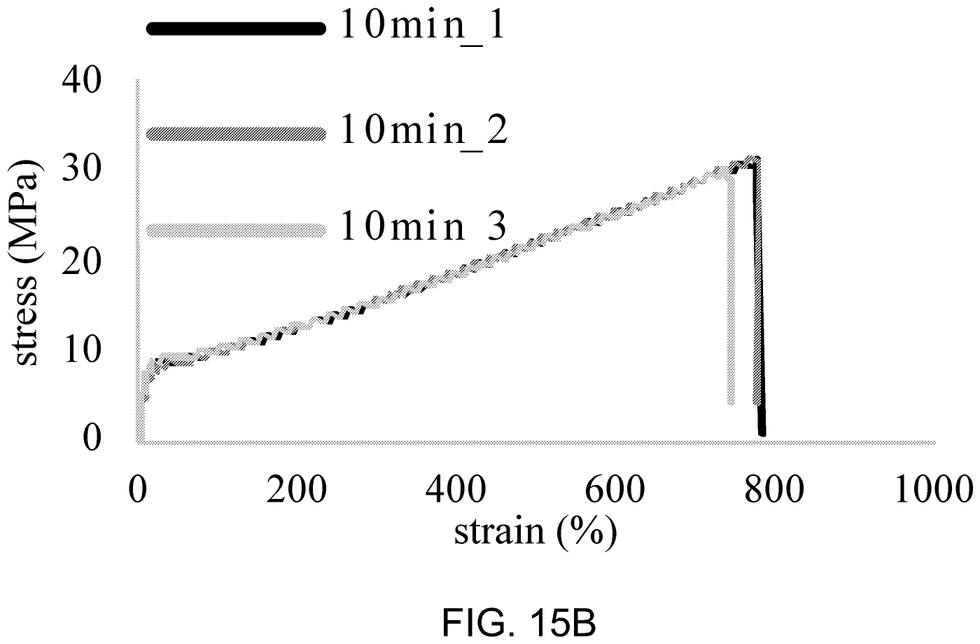

A high molecular weight PMHB sample was selected for tensile testing based on its Tₘ and Mₙ and were melt-pressed into “dogbone” samples. An 80:20 rac-1: rac-2 mixture was polymerized by [ONN]Zn(OⁱPr) using ligand L1-1 ([L1-1]Zn(OⁱPr)) to yield a sample of syndioenriched cis-co-trans PMHB (Mₙ=115 k, Ð=1.03) (FIG. 15A) The sample showed 700-800% elongation at break and high tensile strength (FIG. 15B).

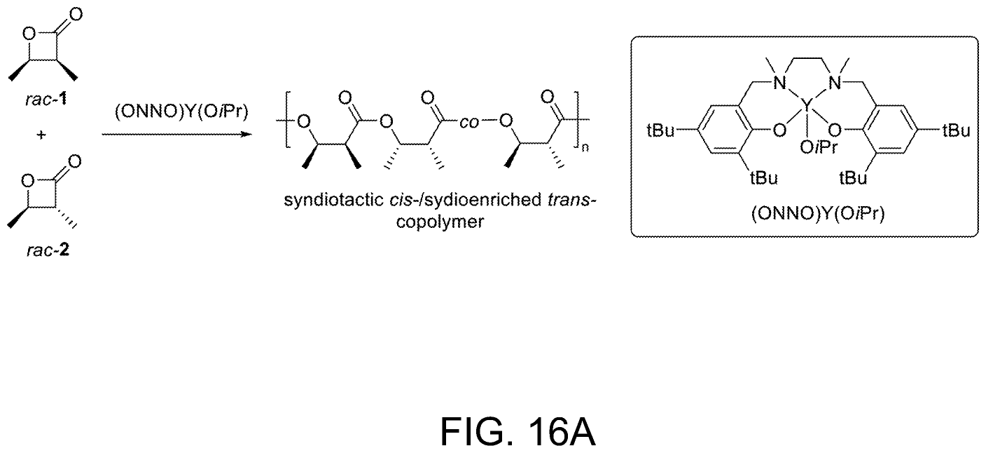

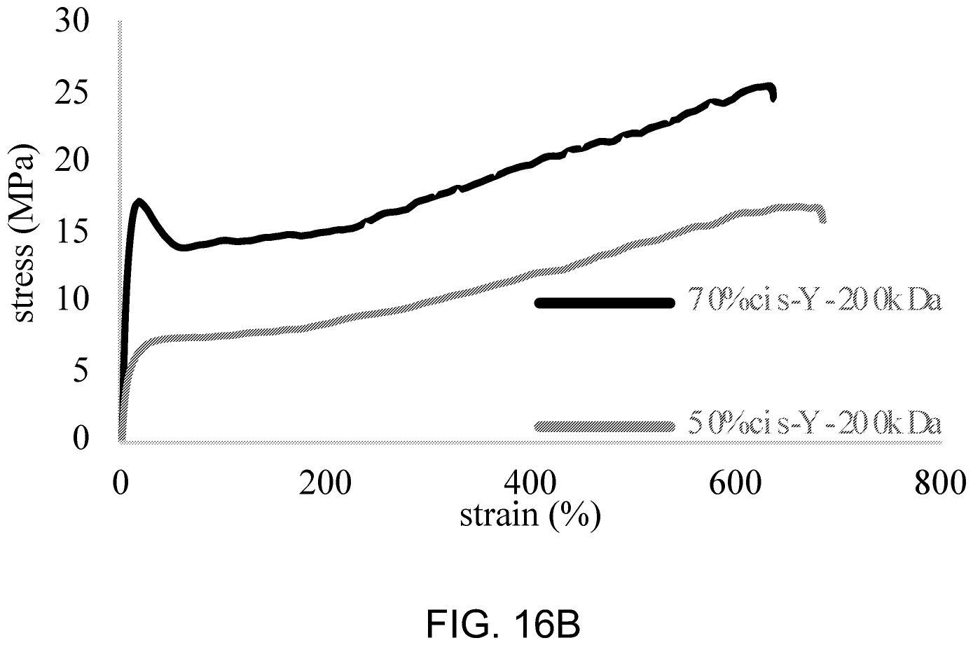

On the other hand, if the syndiotactic catalytic system with [ONNO]Y(OⁱPr) was used (FIG. 16A), promising properties were obtained with 70:30 rac-1: rac-2 copolymer. This sample has a larger Young's modulus than the 80:20 rac-1: rac-2 copolymer sample made from zinc catalysts, potentially due to an increase of tacticity in both cis and trans polymer blocks of the resultant copolymer, which increased its mechanical strength. When further increasing the ratio of trans-monomer 2 to 50:50 rac-1:rac-2, the copolymer sample became softer and exhibited elastomeric properties.

The tensile behavior for some of the samples prepared using [ONN]Zn(OiPr) and [ONN]Y(OiPr) is similar to that of iPP and HDPE, respectively. It is very promising to see such superior tensile properties from the most inexpensive source of monomer precursor. In addition to forming tough, semi-crystalline polyesters, the various microstructures of PMHB may be useful in elastomeric multiblock copolymers, either as block copolymers of the semicrystalline and amorphous microstructures of PMHB, or combined with other polymer blocks (e.g., other polyesters, polyethers, or polycarbonates).

### EXAMPLE 2

The following is an example of PDABL compositions of the present disclosure, methods of making same, and uses same.

A series of methylated polyhydroxyalkanoates: poly(3-hydroxy-2-methylbutyrate)s, is described that are structurally inspired by these naturally occurring polyesters. The introduction of an additional methyl substituent and stereocenter significantly expand the scope of these materials. The cis homopolymers exhibit tacticity-independent crystallinity and superior thermal properties, which allows for the discovery of a series of high-melting, thermally stable, and mechanically tough polymers, and a full range of polyolefin-like properties can be further achieved by tailoring the cis/trans ratio of the repeating units. Moreover, these materials can be synthesized from inexpensive carbon monoxide and 2-butene feedstocks in a scalable manner and they can be chemically recycled or upcycled at their end-of-life. The intrinsic crystallinity, versatile properties, abundant feedstocks, and end-of-life utility of this new family of polyesters will enable a powerful platform for the discovery of sustainable alternatives to polyolefin plastic.

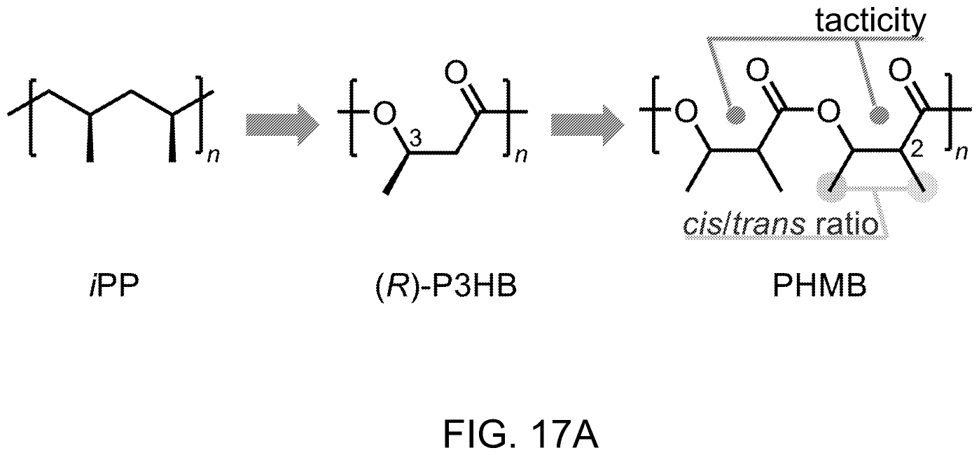

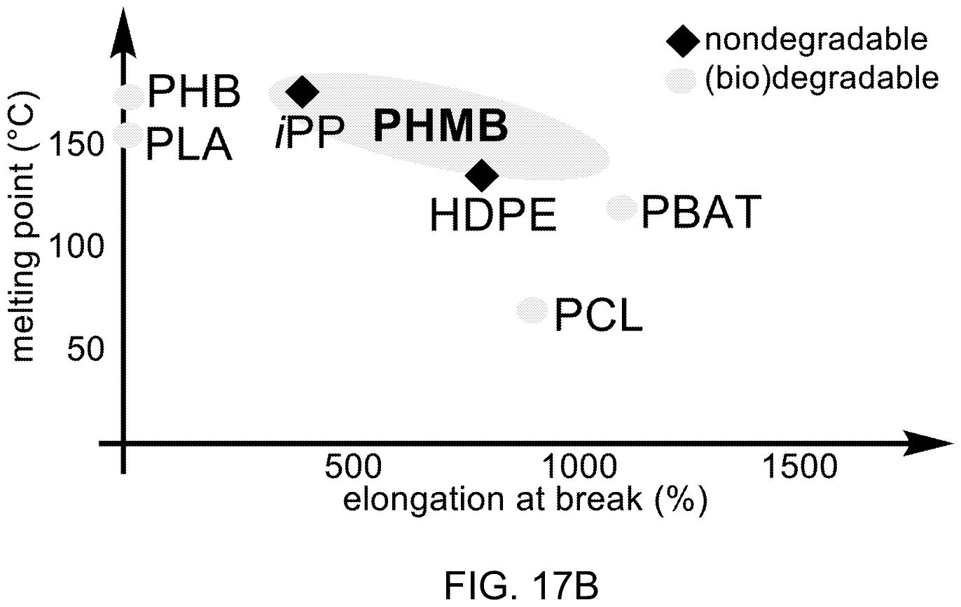

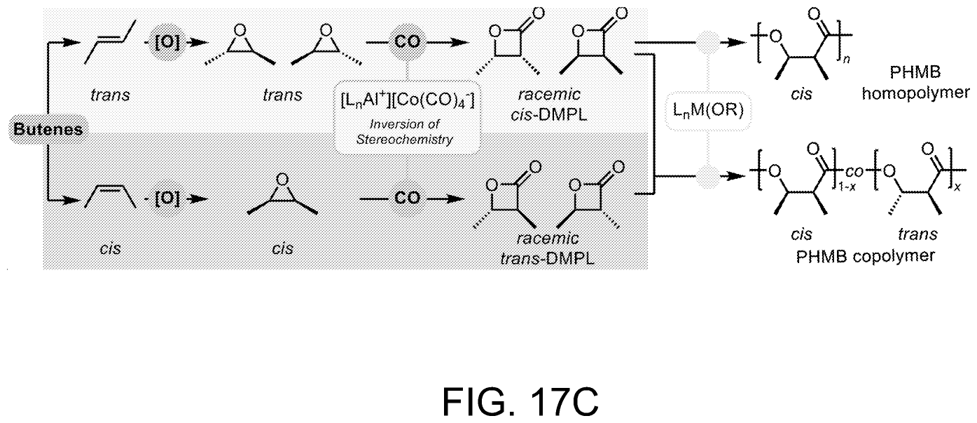

While major efforts were focused on variations at C3 position of repeating units, herein is reported a new class of methylated polyhydroxybutyrates, poly(3-hydroxy-2-methylbutyrate) (PHMB, FIG. 17A), that contain an extra methyl substituent and stereocenter at the C2-position. This structural evolution enables us to establish a versatile platform of PHA polymers with enhanced thermal stability, and their melting points and mechanical strength are close or superior to various commodity plastics (FIG. 17B). Such properties can be further tailored by altering the polymer tacticity and the cis/trans ratio of the monomers.

While the 3-hydroxy-2-methylbutyrate (HMB) repeating unit has been incorporated into P3HB copolymers via ring-opening copolymerization as well as bacterial engineering, PHMB homopolymers have only been reported very recently. While cis- or trans-PHMB homopolymers can be prepared by an efficient one-pot carbonylation/polymerization reaction, the materials were amorphous. The enantiopure (2R, 3R)-trans-PHMB homopolymer can be biosynthesized from gene-modified bacteria and exhibits superior thermal and mechanical properties. However, a systematic evaluation of the full potential of PHMB, especially via chemical synthesis, has not been performed. A strategy was designed that can produce PHMB from 2-butene oxide and CO via carbonylation and polymerization reactions (FIG. 17C). 2-Butene oxide can be industrially produced by the epoxidation of 2-butene, an abundant C4 feedstock that can be derived from both fossil fuel and biorenewable sources. Alternatively, 2-butene oxide can also be prepared via dehydration of microbially produced 2,3-butanediol. Catalytic carbonylation of the epoxides with CO in the presence of [Lewis acid]⁺[Co(CO)₄]⁻ complexes yields 2,3-dimethyl-β-propiolactone (DMPL) with inversion of stereochemistry at the site of ring-opening. With this method, cis- and trans-DMPL can be produced on decagram scale. PHMB can thus be prepared with tunable microstructures from both DMPL isomers in the presence of suitable metal alkoxide catalysts. The homopolymerization of cis-DMPL was initially pursued, as it is derived from the more accessible, thermodynamically more stable trans-2-butene and trans-2-butene oxide feedstocks.

The polymerization of cis-DMPL using complex A was investigated (Table 5). The polymerization was complete within 2 h (h=hour(s)), resulting in high molecular weight (Mₙ>100 kDa) PHMB with excellent syndiotacticity (95% racemo diad, Pᵣ=0.95). The polymer obtained is semicrystalline and exhibits a high Tₘ (>200° C.). While the syndiotactic cis-PHMB's physical properties are promising, the limited availability and high price of yttrium limit the industrial-scale production of such material. Zinc-based catalysts were therefore investigated. It was found that in situ generated complexes B and C both produce cis-PHMB with high molecular weight (Mₙ>100 kDa), narrow dispersity (Ð<1.1), and >99% conversion in 24 hours (h) (Table 6).

TABLE 5

| initiator | time (h) | conv. (%)^{a} | Pᵣ^{b} | Mₙ (kDa)^{c} | Ð^{c} | Tₘ (° C.)^{d, e} | T_{c} (° C.)^{d} |
|---|---|---|---|---|---|---|---|
| A | 2 | >99 | 0.95 | 189 | 1.12 | 204 | 173 |
| B | 24 | >99 | 0.75 | 471 | 1.06 | 191 | 163 |
| C | 16 | >99 | 0.63 | 202 | 1.03 | 186 | 154 |
| PPN[O₂CAd] | 24 | >99 | 0.50 | 21.7 | 1.21 | 171 | 132 |

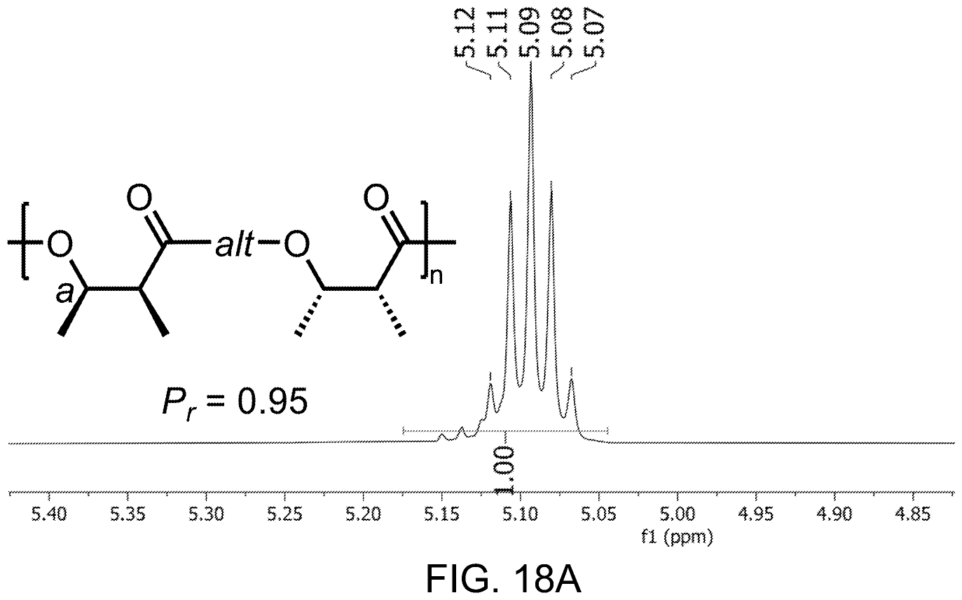

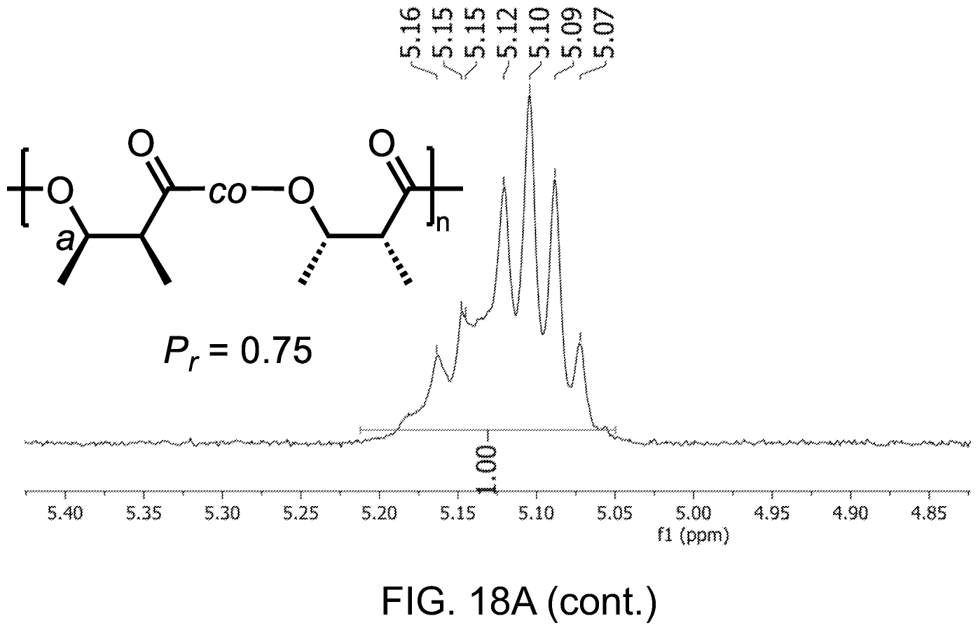

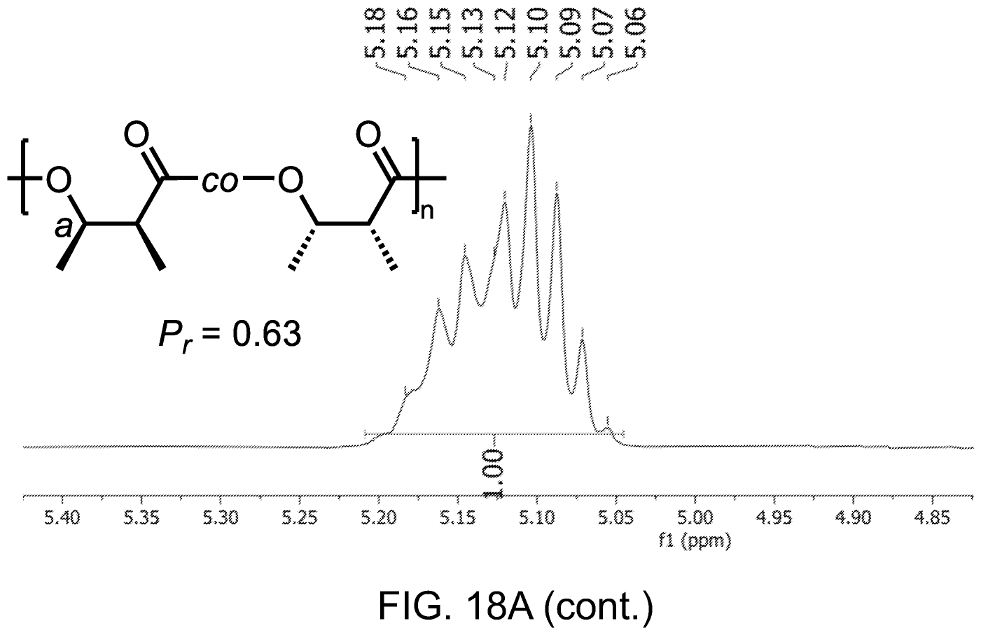

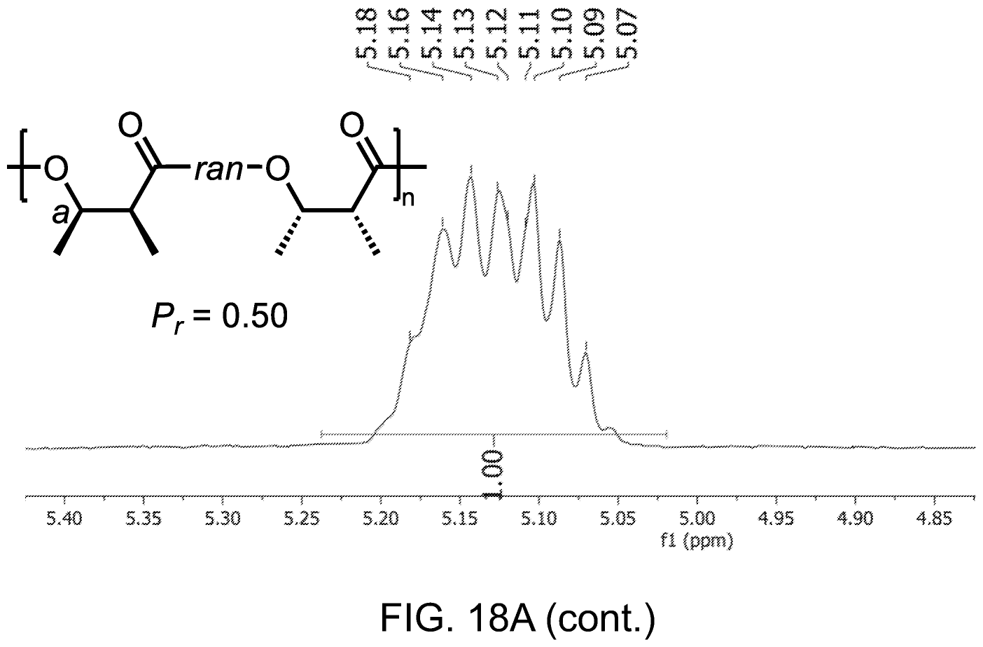

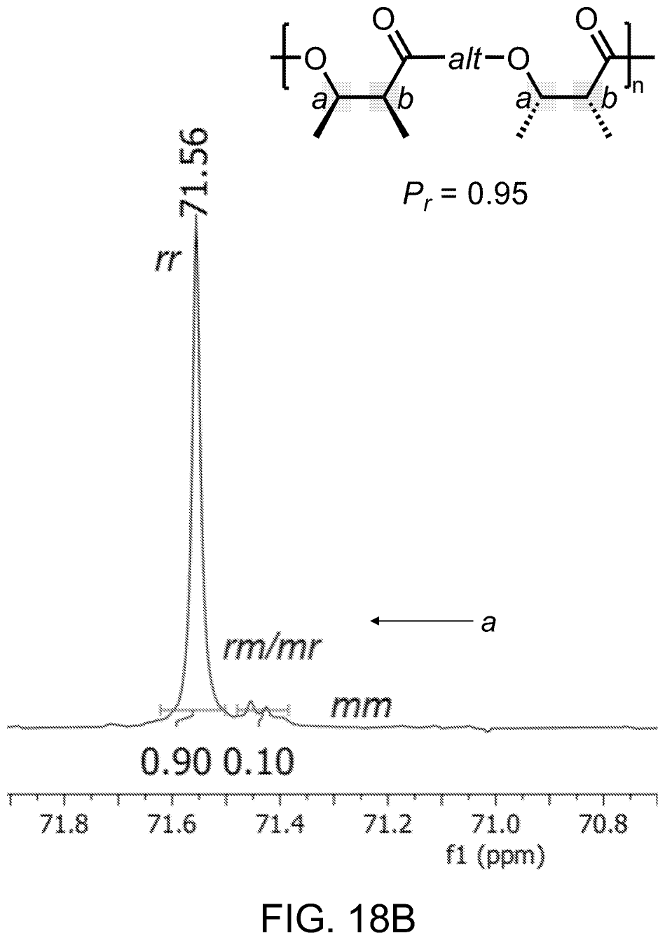

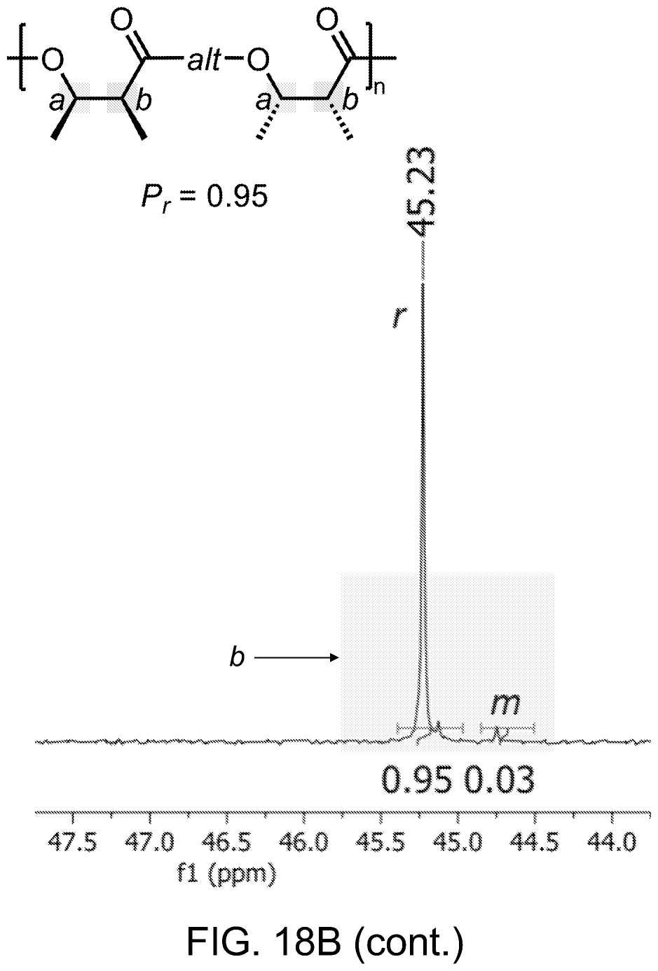

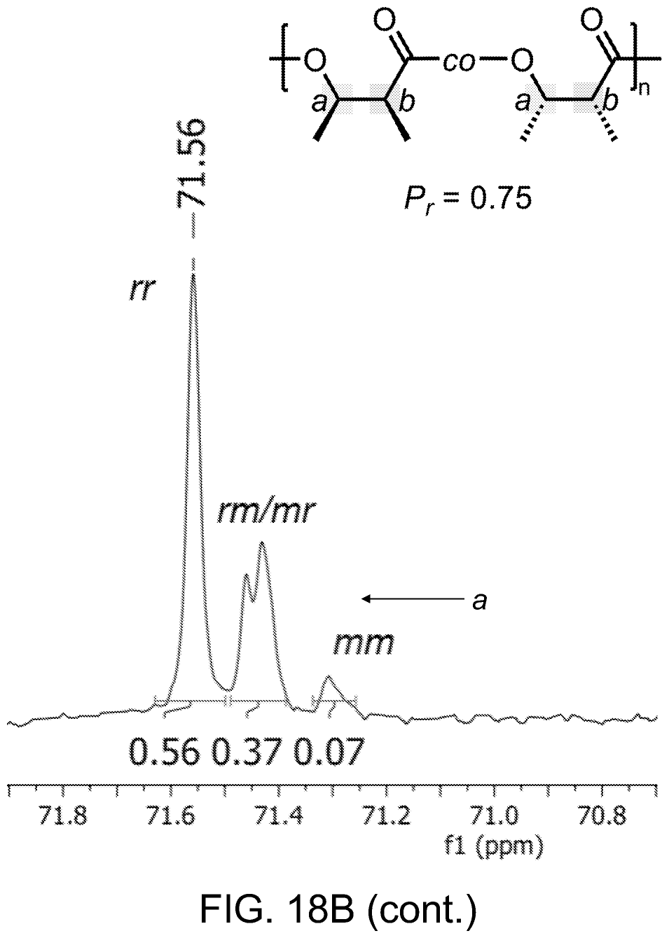

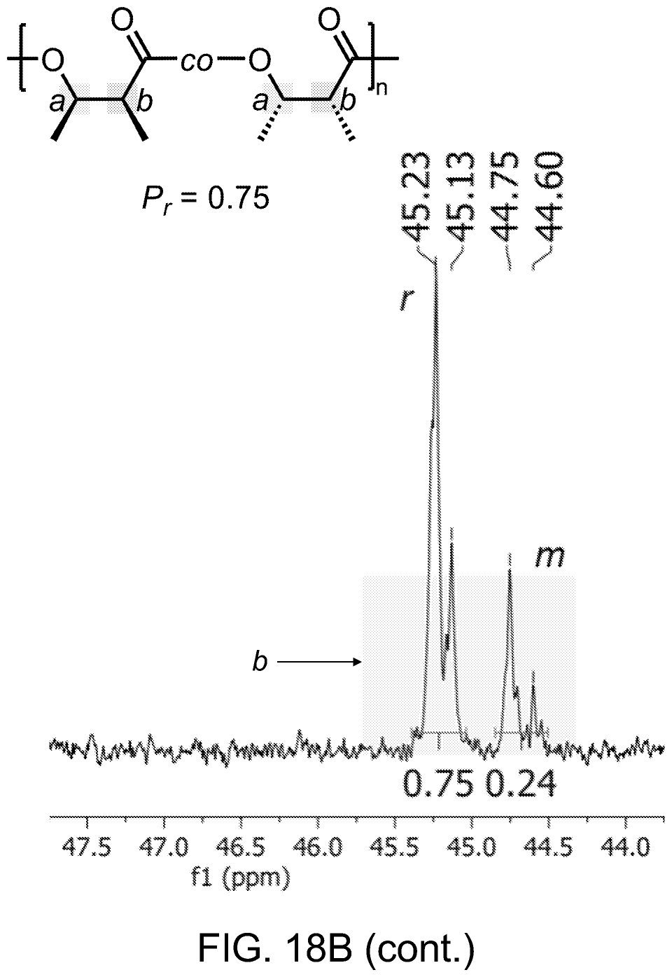

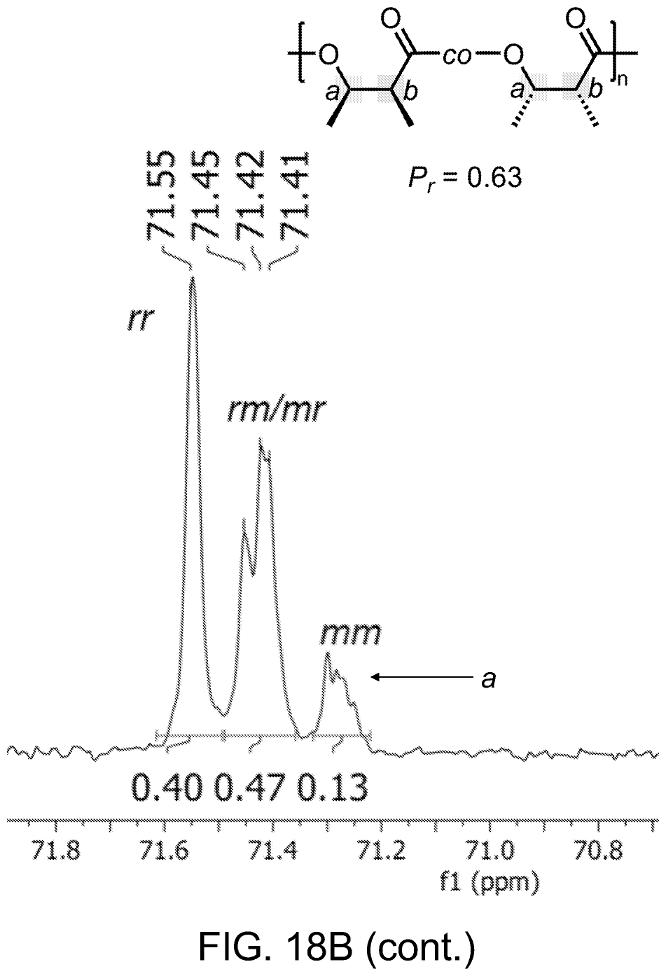

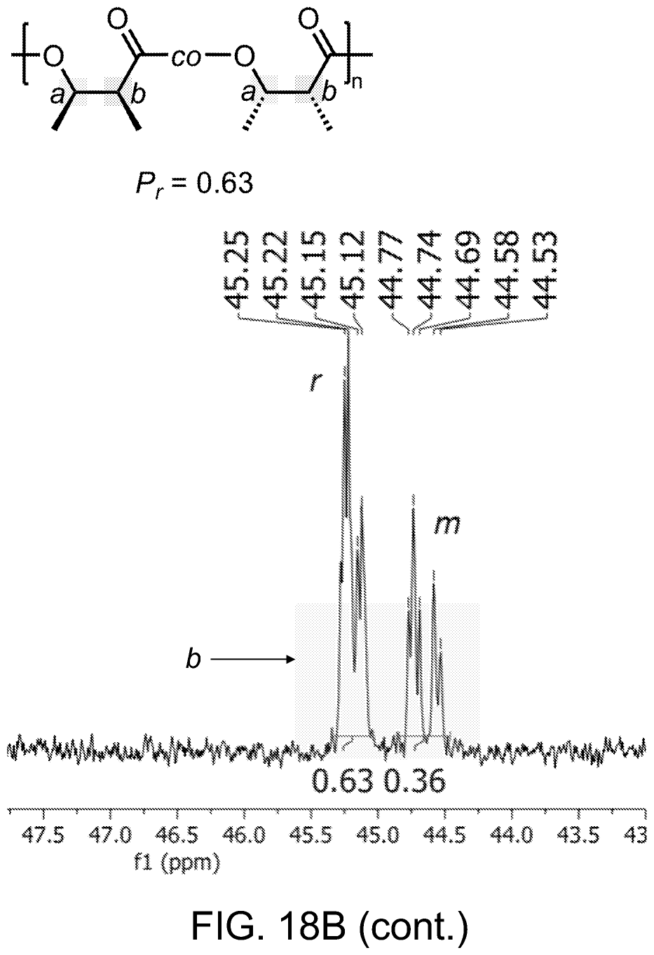

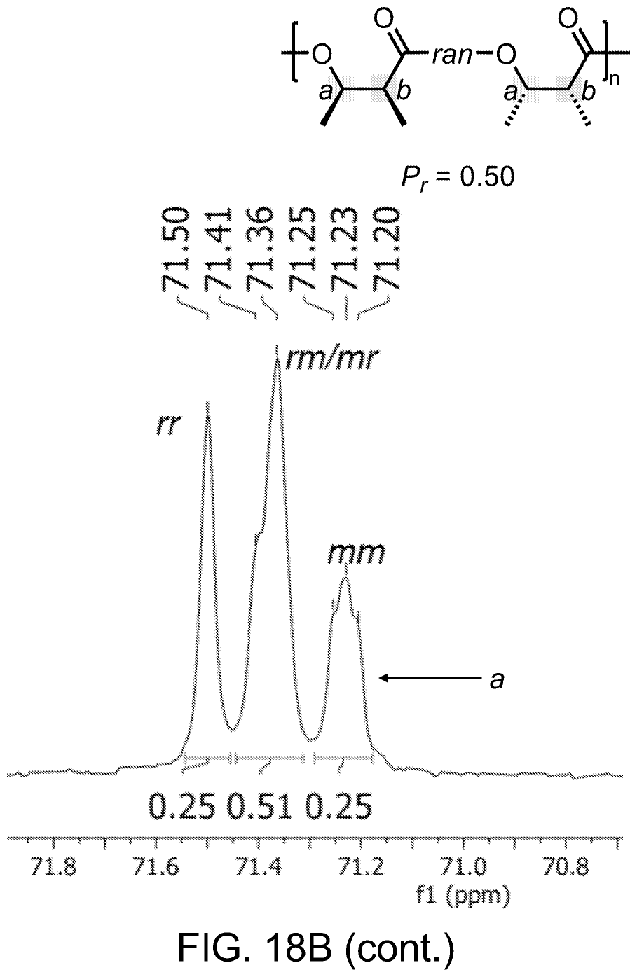

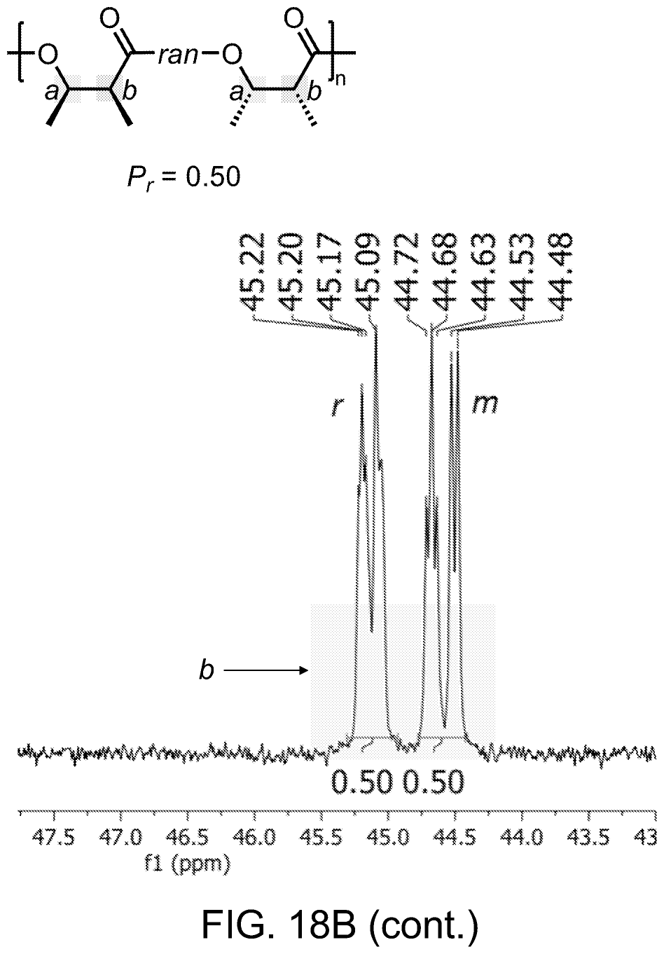

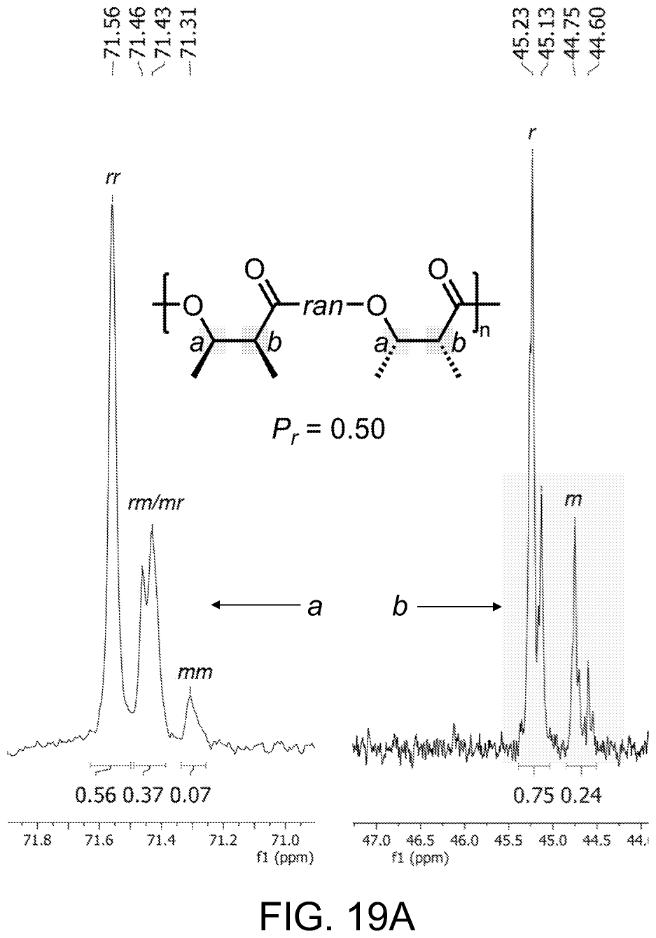

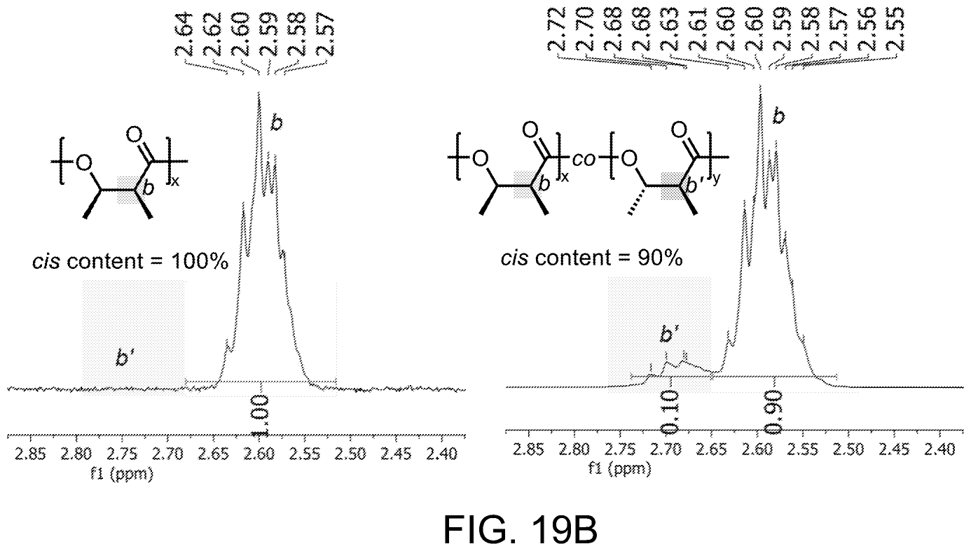

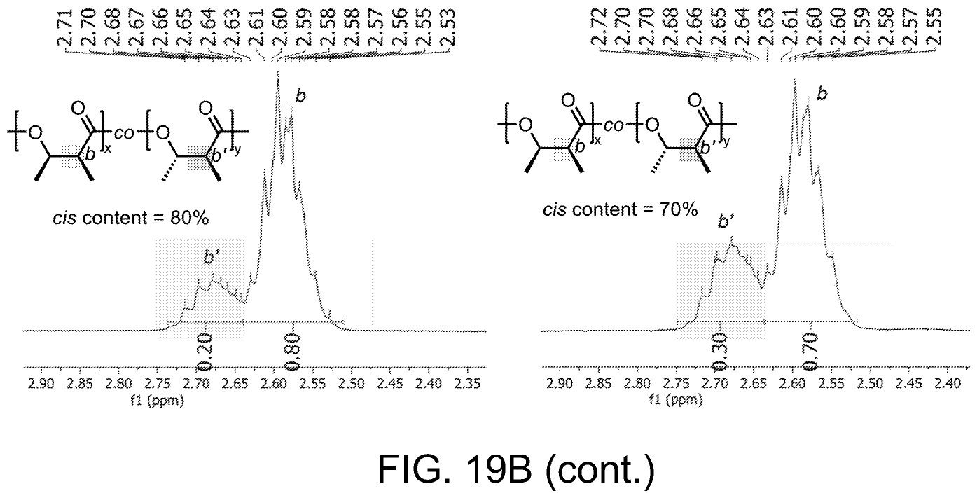

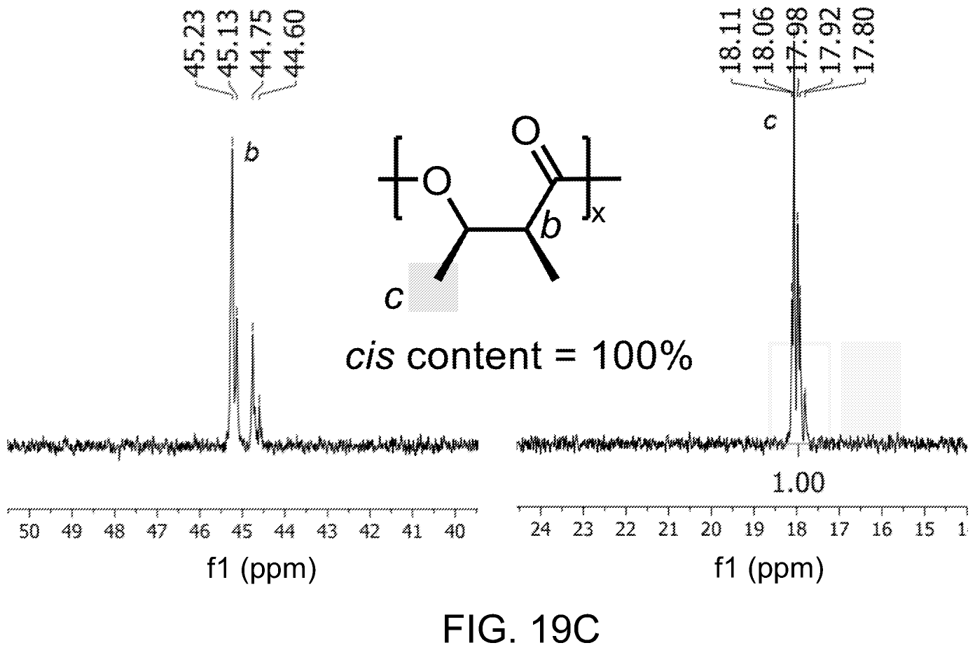

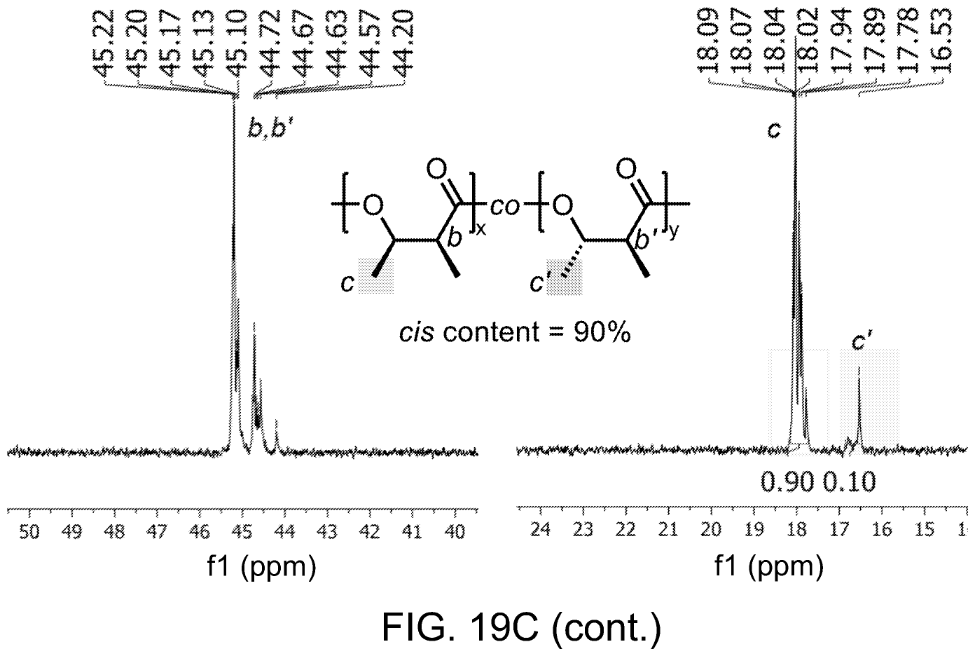

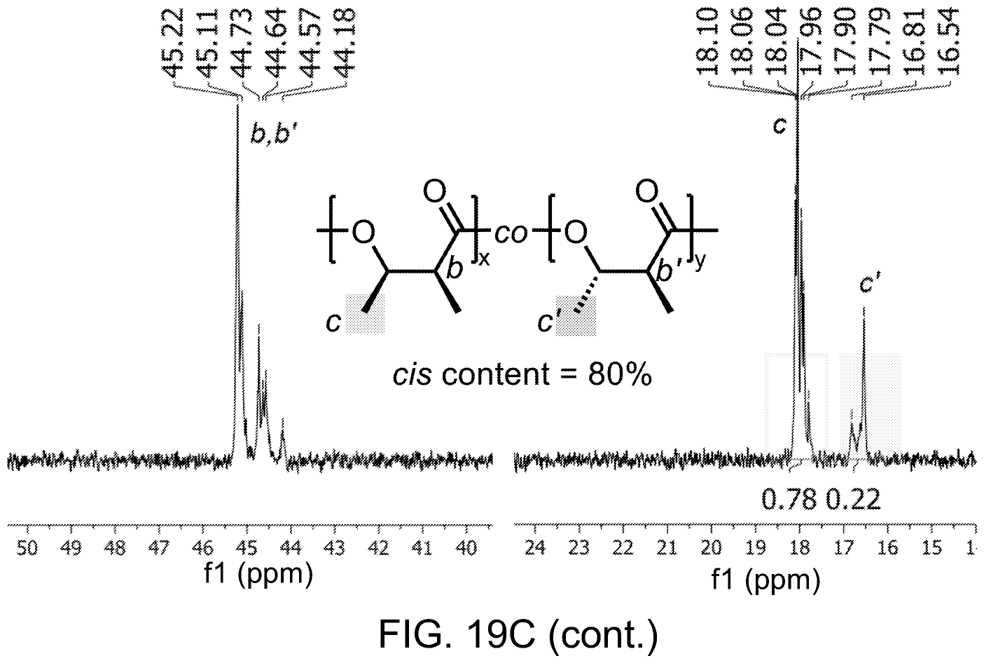

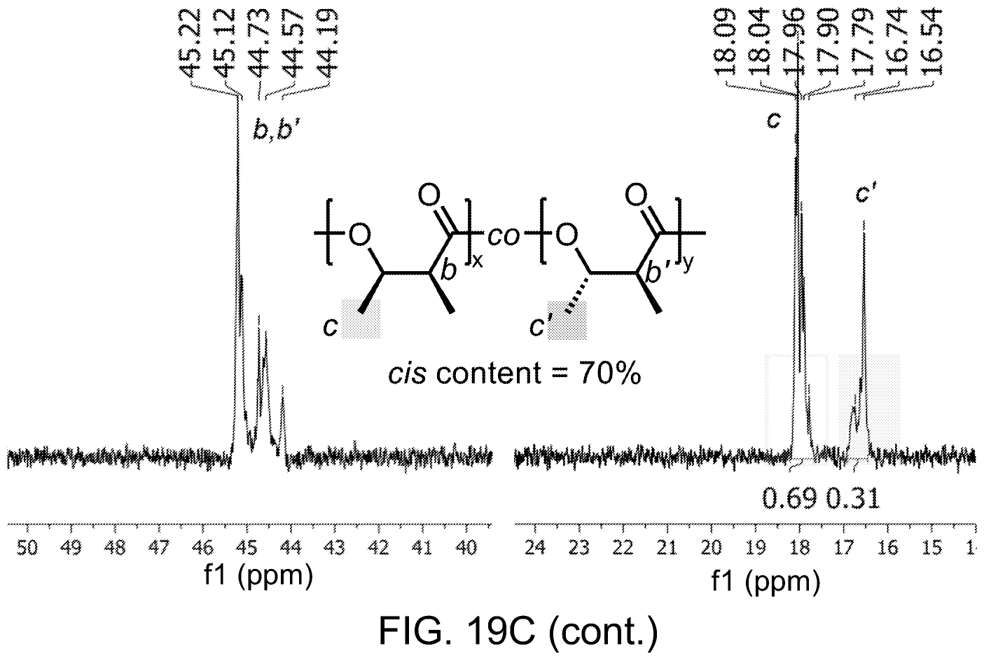

^{a}measured by ¹H NMR of reaction mixture and calculated from the integration of peaks of residual monomer versus the sum of polymer and monomers;
^{b}measured by integrations of ¹³C NMR with peaks around 71 ppm, see FIGS. 18B and 19 for details;
^{c}measured by GPC;
^{d}measured by DSC;
^{e}major melting peak.

TABLE 6

| cis/trans | time | cis conv.^{a} | trans conv.^{a} | Mₙ^{b} | | Tₘ^{c, d} | T_{c}^{c} | T_{g}^{c} |
|---|---|---|---|---|---|---|---|---|
| ratio | (h) | (%) | (%) | (kDa) | Ð^{b} | (° C.) | (° C.) | (° C.) |
| 90/10 | 24 | >99 | 90 | 123 | 1.03 | 176 | 133 | 10 |
| 80/20 | 24 | >99 | 94 | 128 | 1.05 | 160 | 109 | 9 |
| 70/30 | 24 | >99 | 94 | 147 | 1.03 | 139 | 84 | 9 |

^{a}measured by ¹H NMR of reaction mixture and calculated from the integrations of peaks of residual monomer versus the sum of polymer and monomers;
^{b}measured by GPC;
^{c}measured by DSC;
^{d}2^{nd} melting peak.

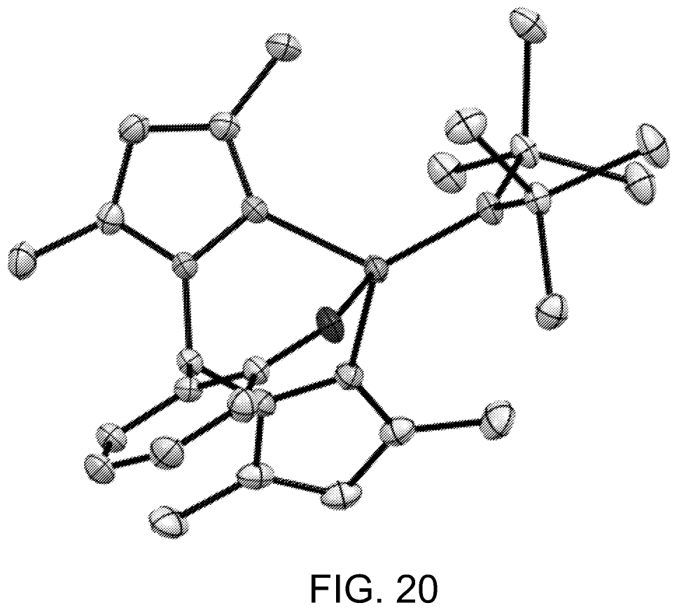

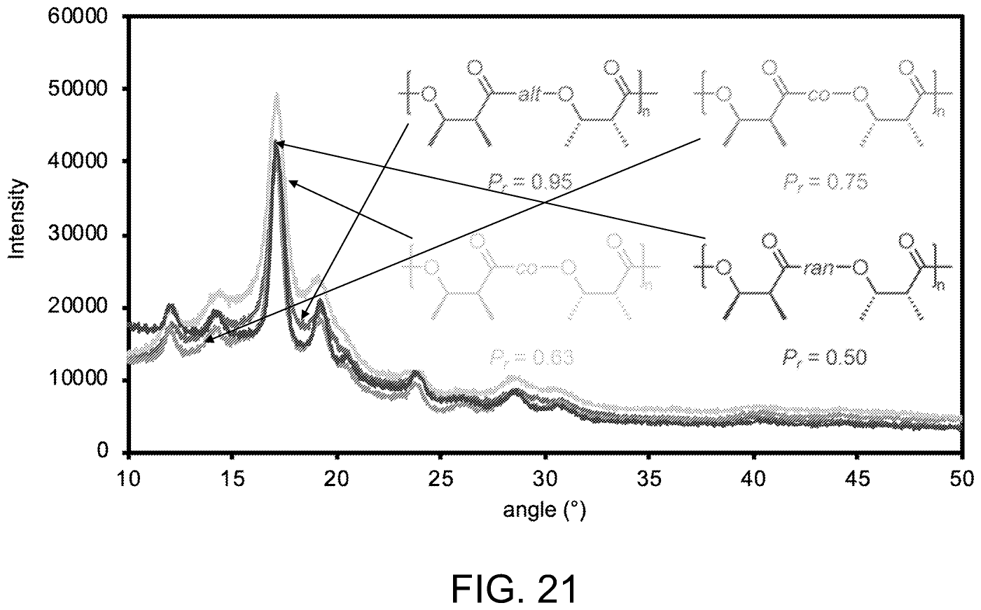

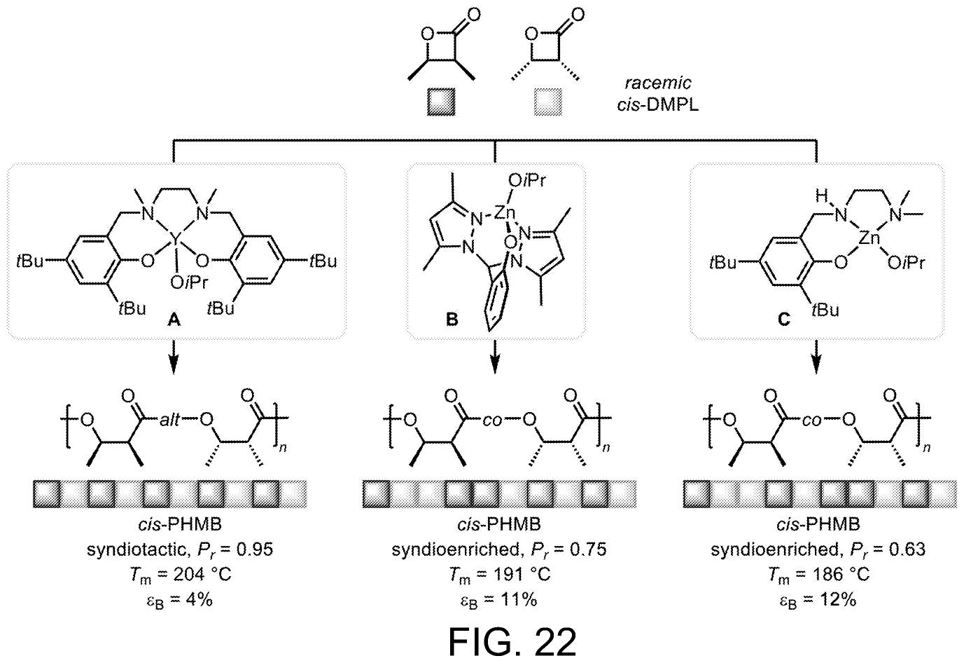

Complex B gives higher syndiotacticity (Pᵣ=0.75), possibly due to the presence of a Cₛ symmetrical bispyrazole-phenoxide ligand backbone (FIG. 20). Complex C induces similar degrees of syndiotacticity (Pᵣ=0.63) in cis-PHMB compared to previous P3HB synthesis⁴⁰ with similar catalysts. Both PHMB polymers, however, are semicrystalline with high Tₘ, and their melting temperatures decrease slightly with deceasing tacticity. Surprisingly, completely atactic cis-PHMB (using PPN[O₂CAd]) is also semicrystalline with a high melting point (FIG. 5). Similar powder X-ray diffraction (PXRD) patterns were observed for all cis-PHMB samples, regardless of stereoregularity (FIG. 21). The tacticity-independent crystallinity and extremely high melting points were unexpected and may permit the use of cis-PHMB as a versatile crystalline component in PHA thermoplastics. However, all these materials are brittle, with strain at break (ε_{B}) not exceeding 20% (FIG. 22). The brittleness across these materials severely limits their further applications and suggests that simply adjusting the tacticity is insufficient to improve their mechanical properties. Thus, other structural modifications were investigated to solve this limitation.

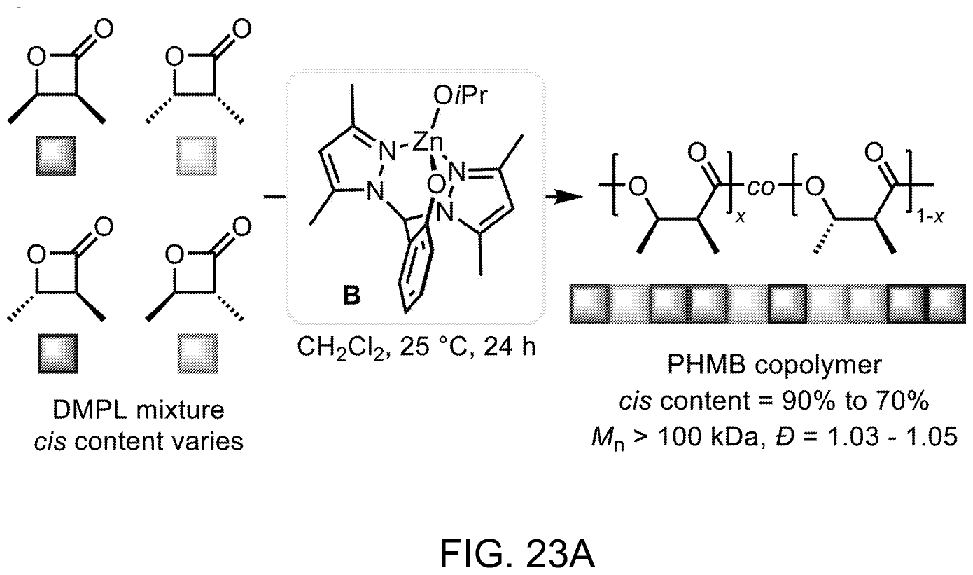

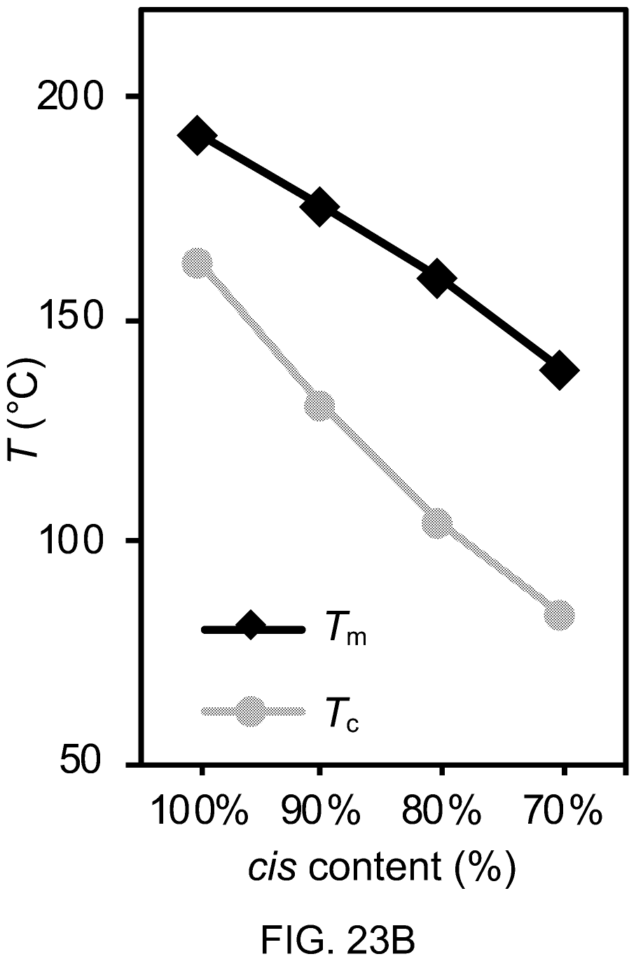

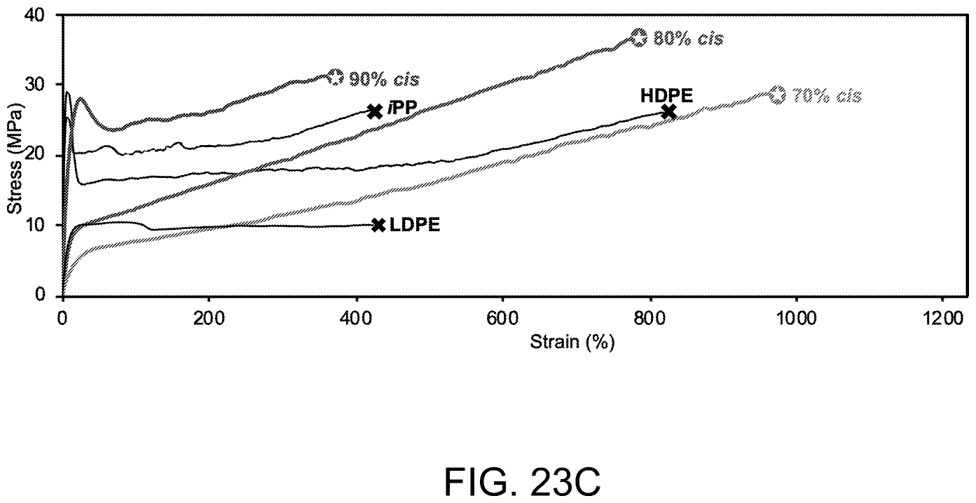

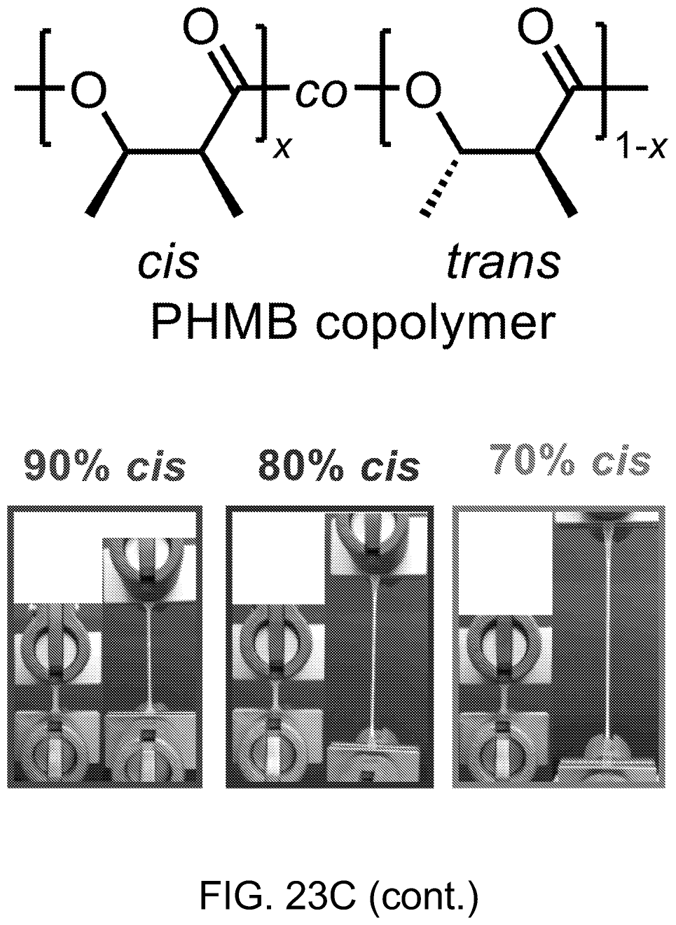

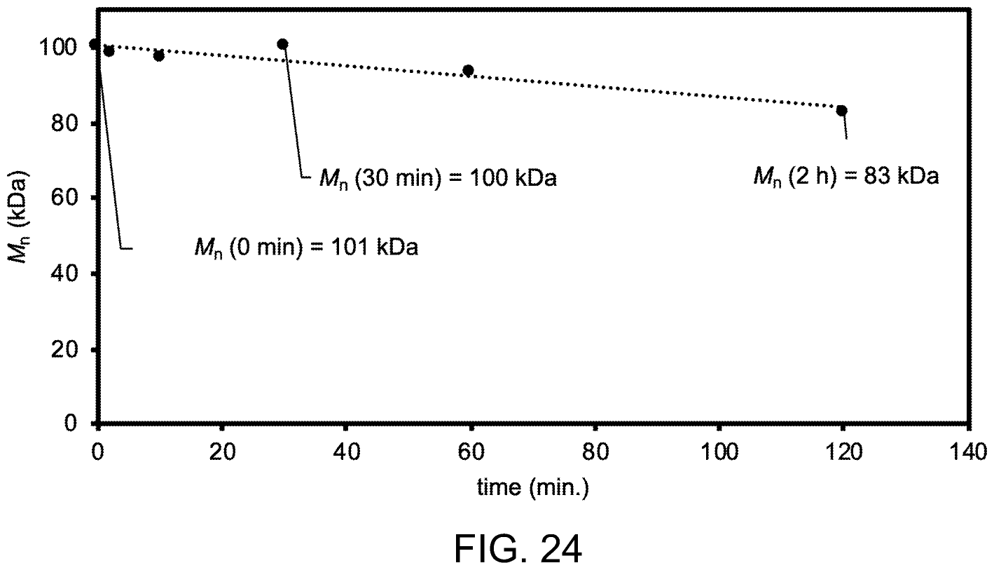

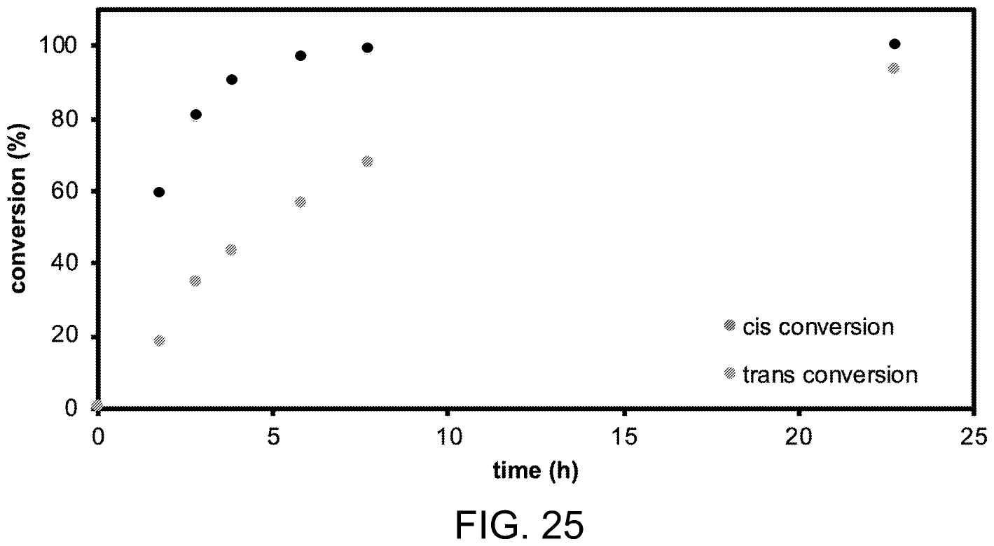

The toughness of R-P3HB can be improved by copolymerization with different comonomers bearing similar chemical structures. With this strategy, trans-DMPL may be an ideal comonomer due to its structural resemblance to the cis isomer. In addition, the comonomer stream could come from a mixture of 2-butene without laborious separation of the two stereoisomers, which would greatly enhance its industrial practicality. Since the copolymer would likely have diminished crystallinity and thermal behaviors, complex B was chosen for further studies, hoping that the higher syndiotacticity it provided in cis-PHMB could offset the decreased Tₘ from stereodefects introduced by the comonomer. When the mixtures of DMPL isomers with varied cis content (90%, 80% and 70%) were polymerized by complex B, high molecular weight copolymers of PHMB were obtained (FIG. 23A and Table 11). All three copolymers exhibit semicrystallinity albeit with moderate tacticity for the major cis composition. Although their Tₘ and T_{c} decrease with lower cis content (FIG. 23B), these copolymers still maintain high melting points (Tₘ>130° C.). Unlike R-P3HB, minimal decrease in molecular weight was observed when the selected PHMB copolymer was heated at 170° C. (FIG. 24). Similar enhancement in thermal stability was also reported for biosynthesized trans-PMHB. Further kinetic experiments showed that the cis-DMPL was consumed faster than the trans isomer (FIG. 25) in the copolymerization, indicating that the copolymers are tapered rather than completely random. Given the lower melting points and attenuated crystallinity of the copolymers, it was expected that the copolymerization strategy might improve the ductility and toughness of the materials.

The tensile properties of the three copolymers (FIG. 23C) were also measured. Encouragingly, all copolymers are tough materials with ε_{B} greater than 300%. At higher cis-DMPL content (90%), the copolymer behaves very similar to iPP and high-density polyethylene (HDPE), with a high yield stress (σ_{Y}=26.1±1.3 MPa) and ductility (ε_{B}=373±50%). Although it is less stiff than the hard polyolefins, with Young's modulus (E)=0.34±0.01 GPa, it possesses a higher stress at break (σ_{B}=30.7±1.3 MPa). Upon lowering the cis content to 80%, the copolymer becomes softer and resembles low-density polyethylene (LDPE) with similar yield stress (σ_{Y}=10.3±0.2 MPa) and Young's modulus (E=0.106±0.003 GPa). Moreover, it is more ductile (ε_{B}=783±7%) and tougher, exhibiting a much higher stress at break (σ_{B}=36.6±0.1 MPa) than LDPE. If the cis-DMPL percentage is further decreased to 70%, the copolymer is even softer and can be stretched to about 10 times of its original length (ε_{B}=1020±55%). The ductility of these copolymers is further illustrated by the photos taken before and after being stretched (FIG. 23C). These results demonstrate that a full spectrum of different polyolefin-like materials, from hard to soft, can be easily obtained by simply adjusting the cis-DMPL content in the comonomer mixture, which can be precisely controlled by the trans/cis ratio of starting 2-butene and 2-butene oxide.

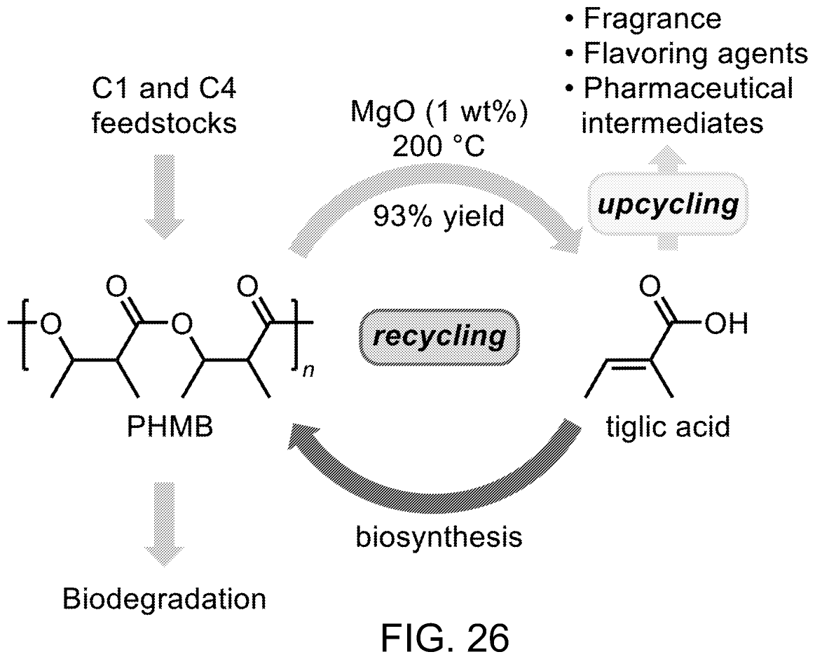

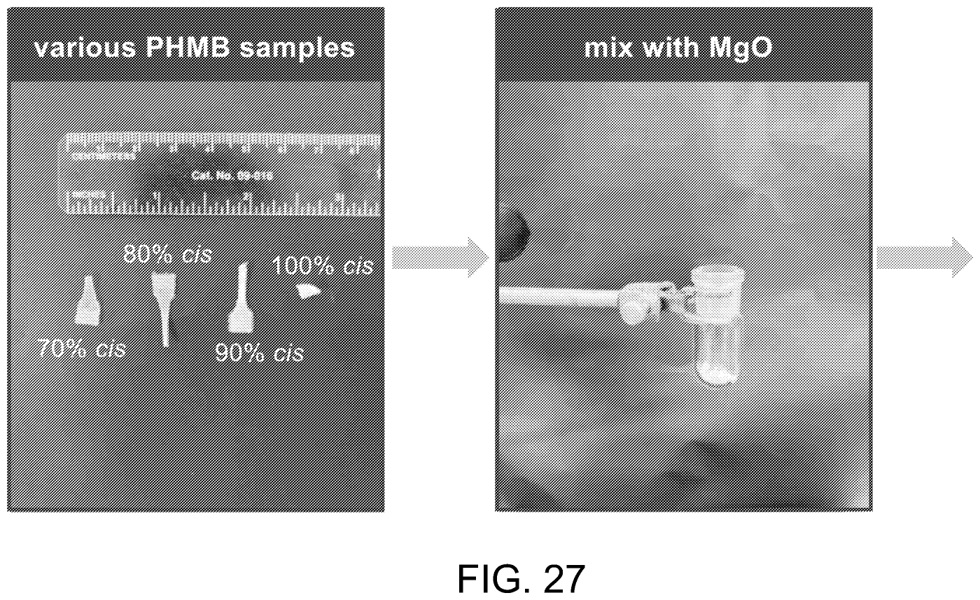

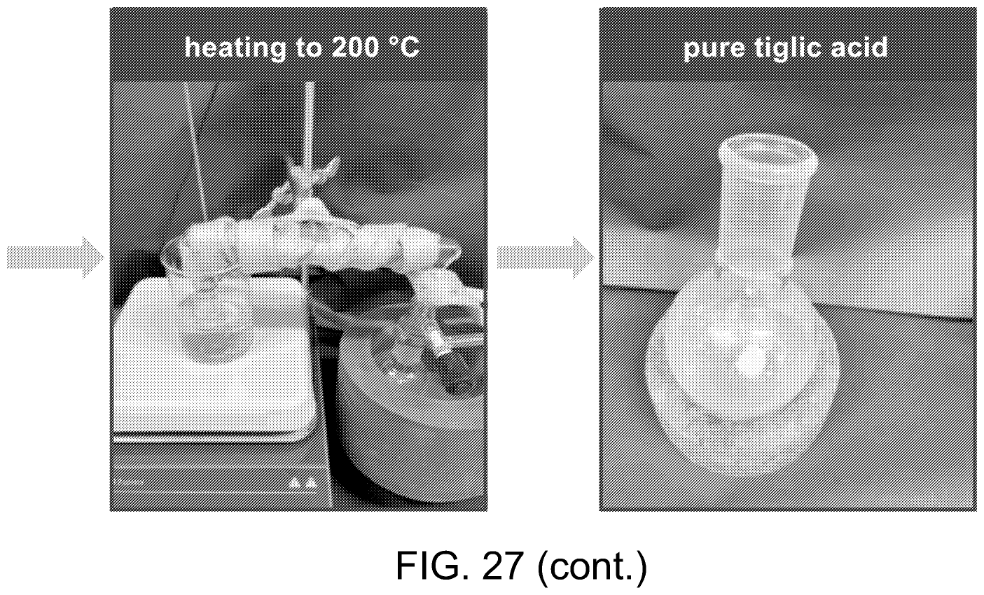

Finally, a preliminary depolymerization study of PHMB to tiglic acid was conducted (FIG. 26). In the presence of MgO (1 wt %), PHMB can be depolymerized cleanly at 200° C. to give pure tiglic acid with 93% yield (FIG. 27). Since tiglic acid was used as a feedstock for the biosynthesis of trans-PMHB, this process offers potential chemical recycling of PHMB. In addition, tiglic acid and its esters have been commonly used in fragrance and food additives, suggesting that PHMB may be also upcycled to value-added products in a green, atom-economic manner. The facile post-consumer transformations of PHMB will permit additional avenues for their waste management.

In conclusion, a new class of PHA polymer, PHMB, was discovered that can be easily synthesized from industrial C4 feedstocks 2-butene and carbon monoxide in a practical and scalable manner, and may biodegrade, be recycled, or upcycled The cis-PHMB homopolymers exhibit tacticity-independent crystallinity and high melting points, and by copolymerizing both cis- and trans-DMPL, a full range of tough and high-melting copolymer materials could be obtained that mimic commodity polyolefin plastics in a well-controlled and predictable manner. These materials could provide a promising platform for sustainable alternatives to polyolefin plastics, and their facile synthesis, renewable sourcing, and potential biodegradability may provide a new way to mitigate plastic pollution.

Materials and Methods. General Considerations. All manipulations of air and water sensitive compounds were carried out under nitrogen in glovebox or by using standard Schlenk line techniques. Solvents used for air-sensitive reactions were purified either over columns of alumina and copper (Q5) (toluene, hexane and THF) or over columns of molecular sieves and alumina [methylene chloride (CH₂Cl₂)], dispensed into an oven-dried Straus flask, degassed via three freeze-pump-thaw cycles, and stored under nitrogen over activated 3 Å molecular sieves in a glovebox. Otherwise, solvents (Et2O, hexanes, MeOH) were used as received. All other chemicals and reagents, except for polymerization materials, were purchased from commercial sources (Aldrich, Oakwood Chemical, Strem, TCI Chemicals, Alfa Aesar, Acros, and Fisher) and used without further purification.

Materials. The cis- and trans-2-butene oxide (97%, Synquest) were dried over CaH₂ for three days and vacuum transferred, degassed via three freeze-pump-thaw cycles, and stored at room temperature under nitrogen. Carbon monoxide (CO) cylinder was purchased from Matheson. iPrOH (99.5%, Sigma-Aldrich, Sure/Seal™) was stored under nitrogen over activated 3 Å molecular sieves. NaCo(CO)₄, TPPAlCl, [(Salph)AlCl(THF)₂][Co(CO)₄], Zn(HMDS)₂, ligand S1 (also referred to herein as ligand L5-3 of Example 1), ligand S2 (also referred to herein as ligand L1-1 of Example 1), complex A-N(DMS)₂ and bis(triphenylphosphine)iminium adamantate (PPN[O₂CAd]) were prepared by literature methods.

Characterization Methods. ¹H and ¹³C NMR—Spectra were recorded on a Bruker AV III HD (1H, 500 MHz) spectrometer with a broad band Prodigy cryoprobe or Varian INOVA 400 (1H, 400 MHZ) spectrometer. Chemical shifts (δ) for ¹H and ¹³C NMR spectra were referenced to protons of the residual solvent (for ¹H) and deuterated solvent itself (for ¹³C). [CDCl₃: 7.26 ppm (¹H), 77.16 ppm (¹³C). CD₂Cl₂: 5.32 ppm (¹H), 53.84 ppm (¹³C)]. Elemental Analysis—Elemental analysis was performed at the University of Rochester using a PerkinElmer Model AD6000 Autobalance and a PerkinElmer 2400 Series II Analyzer. Air-sensitive samples were handled in a VAC Atmospheres glovebox. The sample was transferred under argon and was combusted in a tin capsule that was crimp-sealed with a die apparatus. Gel Permeation Chromatography (GPC)—Analyses were carried out using an Agilent 1260 Infinity GPC System equipped with an Agilent 1260 Infinity autosampler and a refractive index (RI) detector. The Agilent GPC system was equipped with two Agilent PolyPore columns (5 μm, 4.6 mm ID), which were eluted at a rate of 0.3 mL/min with THE containing 0.02 wt. % di-tert-butylhydroxytoluene (BHT) at 30° C. and calibrated using monodisperse polystyrene standards. Power X-ray Diffraction (PXRD)—Power X-ray diffraction experiments of polymers were performed on a Rigaku Ultima IV diffractometer equipped with a Cu Kα source (λ=1.54 Å). X-ray Diffraction—Low—temperature X-ray diffraction data of metal complex were collected on a Rigaku XtaLAB Synergy diffractometer coupled to a Rigaku Hypix detector with Cu Kα radiation (λ=1.54184 Å), from a PhotonJet micro-focus X-ray source at 100 K. The diffraction images were processed and scaled using the CrysAlisPro software. The structures were solved through intrinsic phasing using SHELXT and refined against F² on all data by full-matrix least squares with SHELXL following established refinement strategies. All non-hydrogen atoms were refined anisotropically. All hydrogen atoms bound to carbon were included in the model at geometrically calculated positions and refined using a riding model. The isotropic displacement parameters of all hydrogen atoms were fixed to 1.2 times the Ueq value of the atoms they are linked to (1.5 times for methyl groups). Details of the data quality and a summary of the residual values of the refinements are listed in Tables 7-11. Differential Scanning Calorimetry (DSC)—Differential Scanning Calorimetry analysis was performed on a Mettler Toledo Polymer DSC instrument under a flow of N₂. Polymer samples containing 2-5 mg in crimped aluminum pans were prepared for each run. Unless noted elsewhere, the samples were heated to 210° C. at 10° C./min, then cooled to −70° C. at 10° C./min, and the process was repeated for the second time. The T_{c} value was taken from the first cooling curve and the T_{g} and Tₘ values were taken from the second heating curve, all using the maximum value of the derivative of heat flow with respect to temperature using the STARe software. Compression molding—Compression molding was carried out using a 4120 Hydraulic Unit Carver press and stainless-steel die molds. Stainless steel sheet and Mylar protective sheets were obtained from Carver. All polymer processing was carried out on pristine materials (i.e., no BHT, other antioxidants or additives were added). See below for more details of sample preparation. Uniaxial Tensile Elongation Tests—Uniaxial extension experiments were performed on a Shimadzu Autograph AGS-X series instrument with pneumatic grips and a 500 N load cell using dogbone-shaped samples [ca. 0.5 mm (T)×3 mm (W)×10 mm (L)]. Metal grips were used for tensile testing with a grip pressure of 20 psi and a ramp speed of 10 mm/min. Extension to break tests were performed with three replicates per material to report average values and standard deviations for each set. TrapeziumX version 1.5.1 software was used to analyze the resulting data. The Young's modulus was calculated using the slope of the stress-strain curve from 0 to 2% strain. The reference isotactic polypropylene (iPP, Dow H314-02Z) and high-density polyethylene (HDPE, Dow DMDA8904) samples were obtained from Dow and their tensile data were taken from a previous report¹³. The reference low-density polyethylene (LDPE, Dow 955I) was obtained from Dow and its tensile data was taken from a previous report. Thermogravimetric Analysis (TGA)—Thermal gravimetric analysis was performed on a TA Instruments Q500 Thermogravimetric Analyzer. Analysis was performed on ˜10 mg of a given sample at a heating rate of 10° C./min from 22 to 600° C. under nitrogen gas.

### Synthetic Procedures. Synthesis of cis-DMPL Monomer

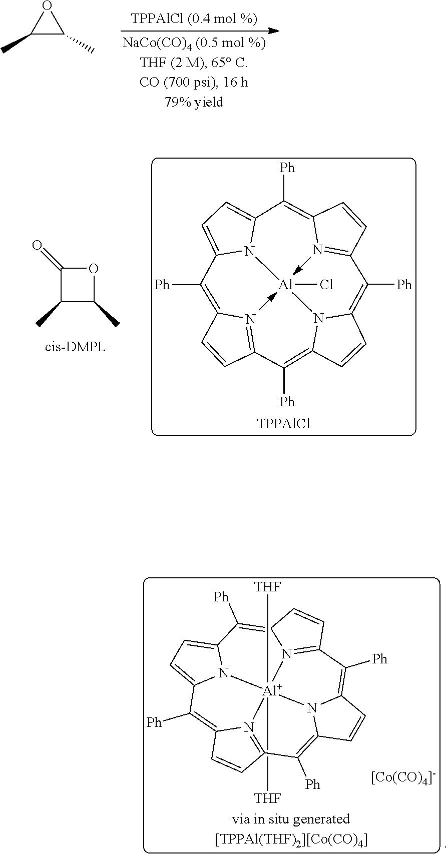

In a nitrogen-filled glovebox, to a 20 mL vial was added meso-tetraphenylporphyrino aluminum chloride (TPPAlCl, 0.521 g, 1.60 mmol 0.004 equiv), NaCo(CO)₄ (0.400 g, 2.00 mmol, 0.005 equiv) and THF (75 mL). The vial was shaken until all the solids dissolved in solution and then the vial was moved into the freezer (−35° C.). To a pre-chilled (−35° C.) Parr reactor was added a stir bar, the above-mentioned chilled solution of catalyst, THF (100 mL) and pre-cooled (−35° C.) trans-2-butene oxide (28.7 g, 399 mmol, 1 equiv). The Parr reactor was then quickly sealed, transferred out from the glovebox, and pressurized and vented with CO for three times. It was then pressurized to 700 psi and moved into a pre-heated oil bath with bath temperature set to 65° C. The reaction was stirred at such temperature for 16 h. After the reaction was finished, the Parr reactor was removed from the oil bath, cooled down to 25° C. and vented. The reaction mixture was then transferred into a round-bottom flask and the residuals in the Parr reactor was rinsed with CH₂Cl₂ and combined. The solvents were removed under reduced pressure on a rotary evaporator with water bath temperature set at 15° C. The crude mixture was then decolorized with activated carbon, dried over CaH₂, and distilled under reduced pressure to give cis-DMPL as a colorless liquid (31.4 g, 79% yield). The spectroscopic data matched with previous reports. The density of cis-DMPL was measured to be d=1.0 g/ml. The freshly distilled cis-DMPL was transferred into the glovebox and was kept in −35° C. freezer, dried over Al₂O₃ beads (BASF F200) for at least one day prior to use for polymerization.

### Synthesis of trans-DMPL Monomer

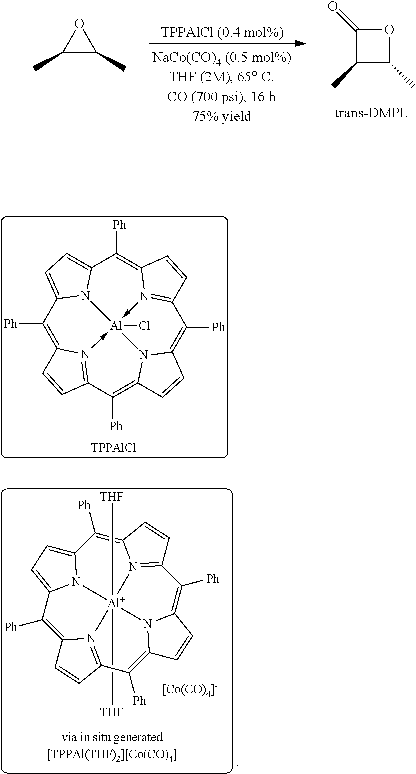

Method A: The synthesis of trans-DMPL is similar to the synthesis of cis-DMPL, except for starting from cis-2-butene oxide (14.6 g, 202 mmol). The crude mixture was then decolorized with activated carbon, dried over CaH₂ and distilled under reduced pressure to give trans-DMPL as a colorless liquid (15.2 g, 75% yield). The spectroscopic data matched with previous reports. The density of trans-DMPL was measured to be d=1.0 g/ml. The freshly distilled trans-DMPL was transferred into the glovebox and was kept in −35° C. freezer, dried over Al₂O₃ beads (BASF F200) for at least one day prior to use for polymerization.

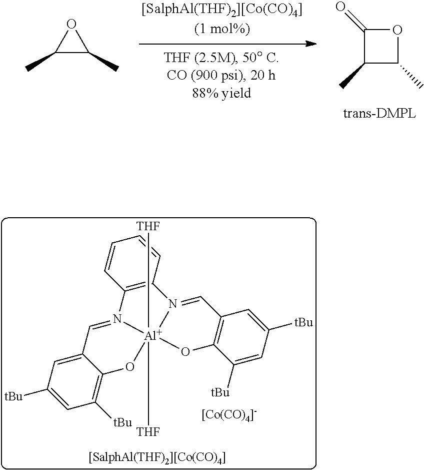

Method B: In a glovebox, [(Salph)Al(THF)₂][Co(CO)₄] (1.39 g, 1.57 mmol, 0.01 equiv) was dissolved in THF (16 mL) and cooled to −35° C. THF (32 mL) and cis-2-butene oxide (16.0 mL, 160 mmol, 1 equiv) were combined and cooled to −35° C. The solutions of catalyst and epoxide were combined in a pre-chilled (−35° C.) Parr reactor and the reactor vessel was sealed and brought out of the glovebox. The reactor was pressurized with CO (900 psi) and heated to 50° C. for 20 h. The reactor was cooled with dry ice and then vented carefully and opened. After removal of THF from the reaction mixture, the lactone was distilled away from catalyst residue under vacuum. A yellow liquid was obtained which was decolorized with activated carbon, dried with CaH₂ and vacuum transferred to give trans-DMPL as a colorless liquid (14.1 g, 88% yield).

### Synthesis of Complex B-HMDS

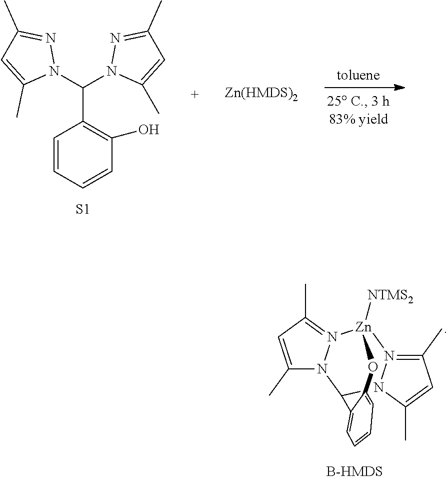

Synthesized according to literature precedence of a related Zn complex. To a stirred suspension of ligand S1 (58.0 mg, 0.200 mmol, 1 equiv) in toluene (4 mL) was added Zn(HMDS)₂ (79.0 μL, 0.200 mmol, 1.00 equiv) dropwise at 25° C. The mixture turned clear, and the stirring was continued for another 2 h. The solvent was then removed to give complex B-HMDS as light-yellow solid (84.6 mg, 83% yield). Recrystallization from toluene at −35° C. gave colorless crystals that were suitable for X-ray diffraction analysis. B-HMDS: ¹H NMR (500 MHZ, CD₂Cl₂) δ 7.05 (ddd, J=8.6, 7.1, 1.9 Hz, 1 H), 6.96 (dd, J=7.6, 1.9 Hz, 1 H), 6.86 (s, 1 H), 6.62 (dd, J=8.4, 1.3 Hz, 1 H), 6.33 (td, J=7.3, 1.3 Hz, 1 H), 6.00 (s, 2 H), 2.47 (s, 6 H), 2.47 (s, 6 H), 0.09 (s, 12 H). ¹³C NMR (126 MHZ, CD₂Cl₂) δ 166.78, 150.96, 141.37, 132.28, 130.86, 124.84, 120.80, 112.61, 107.19, 73.33, 53.84 (p, CD₂Cl₂), 14.11, 11.81, 5.25. Elemental Analysis: calculated (found) for C₂₃H₃₇N₅OSi₂Zn: C, 53.01 (52.56); H, 7.16 (6.95); N, 13.44 (13.24).

### Homopolymerization of cis-DMPL

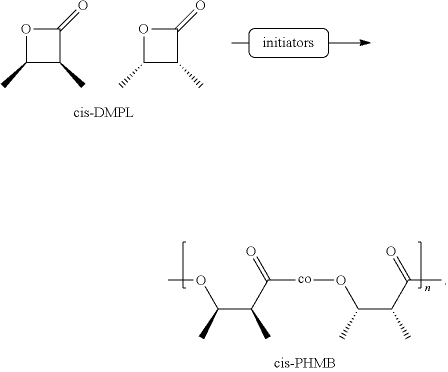

### Synthesis of cis-PHMB Homopolymer With Complex A (Pᵣ=0.95)

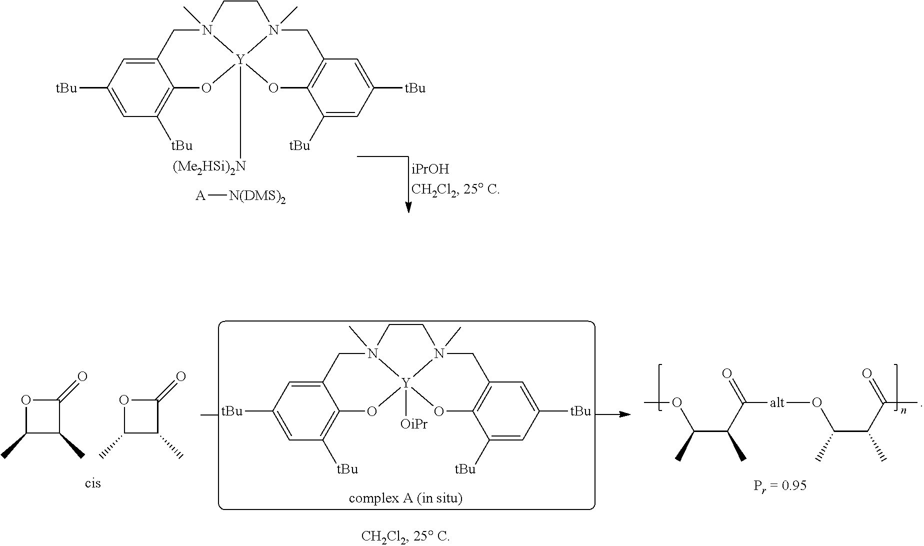

To a stirred solution of complex A-N(DMS)₂ (0.7 mg, 0.001 mmol, 1 equiv) in CH₂Cl₂ (0.39 mL) was added a solution of iPrOH (10 μL, 0.1 M in CH₂Cl₂, 0.001 mmol, 1.0 equiv) at 25° C. to generate complex A in situ. cis-DMPL (0.100 mL, 1.00 mmol, 1000 equiv) was then added to the solution of complex A at 25° C., and the resulting mixture was set at 25° C. for 2 h. The reaction mixture was then removed from glovebox, precipitated from hexane/diethyl ether (2:1 v/v, 300 mL) and dried under vacuum to give the polymer as white foam (0.088 g, 88% yield). GPC Mₙ=189 kDa, Ð=1.12 (the sample had poor solubility in THF and needed to be dissolved in CH₂Cl₂ first, dried briefly and boiled in THF for re-dissolution. The THF solution was measured immediately after filtration to prevent re-precipitation of the polymer). ¹H NMR (500 MHZ, CDCl₃) δ 5.09 (p, J=6.4 Hz, 1 H), 2.59 (p, J=7.0 Hz, 1 H), 1.23 (d, J=6.3 Hz, 3 H), 1.17 (d, J=7.0 Hz, 3 H). ¹³C NMR (126 MHZ, CDCl₃) δ 172.74, 71.56, 45.23, 18.06, 13.13. DSC Tₘ=192° C., 204° C., T_{c}=173° C. (DSC cycle between −70° C. to 220° C. instead of 210° C. due to the higher melting point of the sample). TGA T_{d, 5%}=275° C.

### Synthesis of cis-PHMB Homopolymer With Complex B (Pᵣ=0.75)

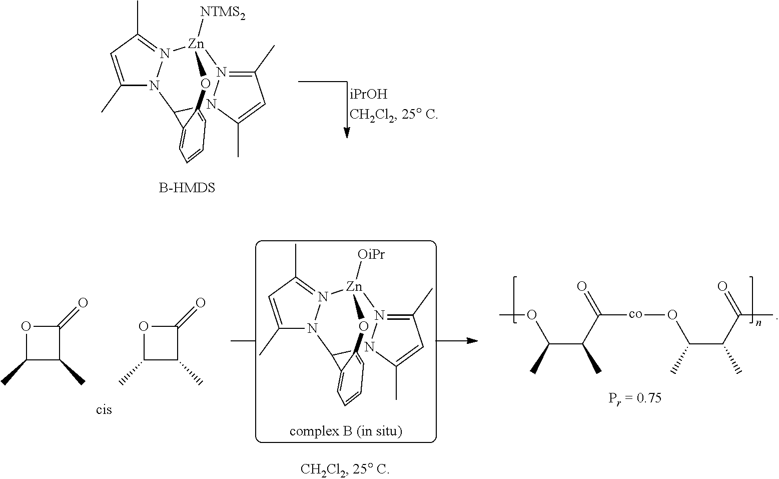

To a stirred solution of complex B-HMDS (5.8 mg, 0.011 mmol, 1 equiv) in CH₂Cl₂ (1.00 mL) was added a solution of iPrOH (0.110 mL, 0.1 M in CH₂Cl₂, 0.0110 mmol, 1.00 equiv) at 25° C. to generate complex B in situ (also referred to herein as [L1-1]Zn(OⁱPr) in Example 1). An aliquot of complex B solution (0.160 mL, 0.01 M in CH₂Cl₂, 1.60 μmol, 1.00 equiv) was added to a solution of cis-DMPL (0.400 mL, 4.00 mmol, 2500 equiv) in CH₂Cl₂ (1.44 mL) at 25° C., and the resulting mixture was set at 25° C. for 24 h. The reaction mixture was then removed from glovebox, precipitated from hexane/diethyl ether (2:1 v/v, 300 mL) and dried under vacuum to give the polymer as white foam (0.370 g, 92% yield). GPC Mₙ=471 kDa, Ð=1.06 (the sample had poor solubility in THF and needed to be dissolved in CH₂Cl₂ first, dried briefly and boiled in THF for re-dissolution. The THF solution was measured immediately after filtration to prevent re-precipitation of the polymer). ¹H NMR (400 MHZ, CDCl₃) δ 5.16-5.07 (m, 1 H), 2.64-2.57 (m, 1 H), 1.25-1.23 (m, 3 H), 1.19-1.17 (m, 3 H). ¹³C NMR (126 MHZ, CDCl₃) δ 172.81, 172.75, 172.73, 172.70, 71.56, 71.46, 71.43, 71.31, 45.23, 45.13, 44.75, 44.60, 18.11, 18.06, 17.98, 17.92, 13.24, 13.12, 12.93, 12.82, 12.76, 12.63, 12.33. DSC Tₘ=183° C., 191° C., T_{c}=163° C. TGA T_{d, 5%}=273° C.

### Synthesis of cis-PHMB Homopolymer With Complex C (Pᵣ=0.63)

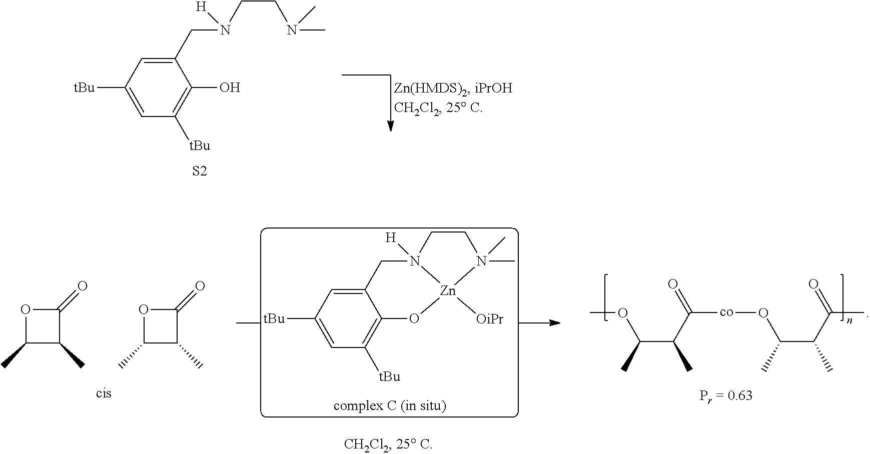

To an 8 mL vial equipped with a stir bar was added ligand S2 (also referred to herein as ligand L1-1 of Example 1) (0.6 mg, 0.002 mmol, 1 equiv) and CH₂Cl₂ (1.93 mL). Upon full dissolution of S2, a solution of Zn(HMDS)₂ (20 μL, 0.1 M in toluene, 0.0020 mmol, 1.0 equiv) was added at 25° C. After stirring for 10 min, a solution of iPrOH (20 μL, 0.1 M in CH₂Cl₂, 0.0020 mmol, 1.0 equiv) was added at 25° C. to generate complex C in situ. To this solution was added cis-DMPL (0.530 mL, 5.30 mmol, 2650 equiv) at 25° C., and the resulting mixture was set at 25° C. for 24 h. The reaction mixture was then removed from glovebox, precipitated from hexane/diethyl ether (2:1 v/v, 300 mL) and dried under vacuum to give the polymer as white foam (0.477 g, 90% yield). GPC Mₙ=202 kDa, Ð=1.03. ¹H NMR (400 MHZ, CDCl₃) δ 5.18-5.06 (m, 1 H), 2.63-2.55 (m, 1 H), 1.24-1.23 (m, 3 H), 1.19-1.16 (m, 3 H). ¹³C NMR (126 MHz, CDCl₃) δ 172.81, 172.75, 172.73, 172.70, 172.67, 71.55, 71.45, 71.42, 71.41, 71.30, 45.25, 45.22, 45.15, 45.12, 44.77, 44.74, 44.69, 44.58, 44.53, 18.12, 18.10, 18.07, 18.05, 17.97, 17.92, 17.89, 17.79, 13.24, 13.12, 13.10, 12.93, 12.86, 12.82, 12.80, 12.75, 12.62, 12.32. DSC Tₘ=176° C., 186° C., T_{c}=154° C. TGA T_{d, 5%}=269° C.

### Synthesis of cis-PHMB Homopolymer From trans-DMPL (Pᵣ=0.50)

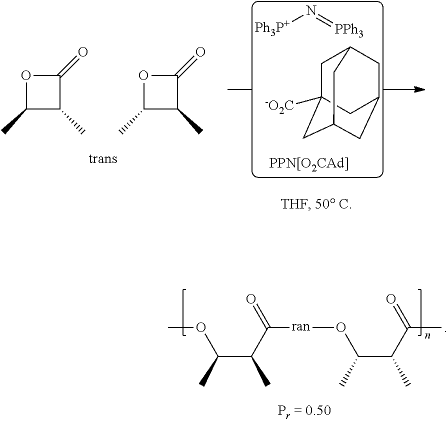

To an 8 mL via equipped with a stir bar was added PPN[O₂CAd] (6.7 mg, 0.0097 mmol, 1 equiv) and trans-DMPL (2 M in THF, 0.500 mL, 1.00 mmol, 103 equiv), and the resulting mixture was heated to 50° C. and stirred for 24 h. The reaction mixture was then removed from glovebox and washed with MeOH (8 mL×3) and Et₂O (8 mL×3) to give the polymer as white powder (0.082 g, 82%). GPC Mₙ=9.2 k, Ð=1.22. ¹H NMR (400 MHz, CDCl₃) δ 5.18-5.07 (m, 1 H), 2.62-2.56 (m, 1 H), 1.24-1.22 (m, 3 H), 1.18-1.16 (m, 3 H). ¹³C NMR (126 MHz, CDCl₃) δ 172.76, 172.71, 172.69, 172.65, 172.62, 71.50, 71.41, 71.36, 71.25, 71.23, 71.20, 45.22, 45.20, 45.17, 45.09, 44.72, 44.68, 44.63, 44.53, 44.48, 18.07, 18.05, 18.01, 17.92, 17.90, 17.86, 17.83, 17.73, 13.18, 13.07, 13.04, 12.87, 12.80, 12.77, 12.59, 12.56, 12.30, 12.26, 12.23, 12.19. DSC Tₘ=149° C., 161° C., 171° C. (3 broad peaks observed), T_{c}=132° C. (DSC cycle between −70° C. to 200° C. instead of 210° C. due to the lower melting point of the sample).

### Copolymerization of cis- and trans-DMPL

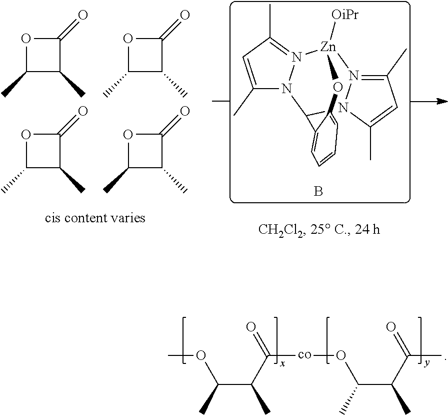

### Synthesis of Copolymer (cis Content=90%) With Complex B (Pᵣ=0.75 for cis-DMPL)

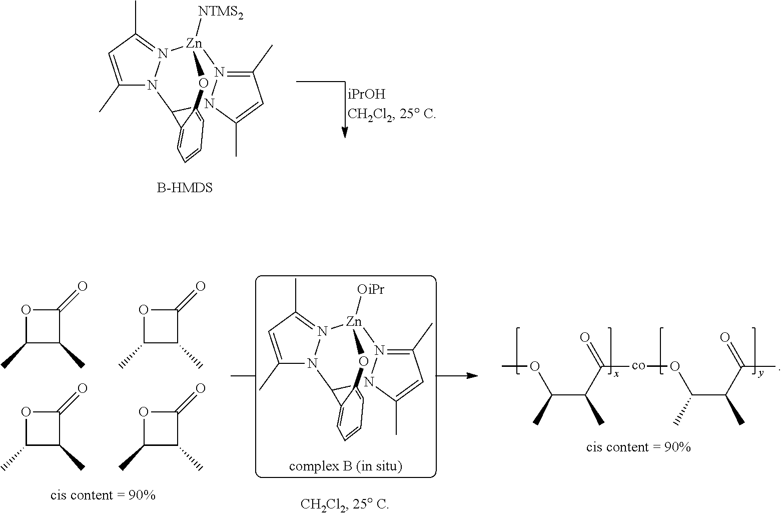

To a solution of cis monomer (0.900 mL, 9.00 mmol, 1260 equiv) and trans monomer (0.100 mL, 1.00 mmol, 140 equiv) in CH₂Cl₂ (0.80 mL) was added a solution of complex B (prepared as described above, 0.178 mL, 0.04 M in CH₂Cl₂, 7.12 μmol, 1 equiv) at 25° C., and the resulting mixture was set at 25° C. for 24 h. The reaction mixture was then removed from glovebox, precipitated from hexane/diethyl ether (2:1 v/v, 500 mL) and dried under vacuum to give the polymer as white foam (0.804 g, 80% yield). GPC Mₙ=123 kDa, Ð=1.03. ¹H NMR (400 MHZ, CDCl₃) δ 5.17-5.05 (m, 1 H), 2.72-2.55 (m, 1 H), 1.24-1.12 (m, 6 H). ¹³C NMR (126 MHz, CDCl₃) δ 172.78, 172.72, 172.70, 172.67, 172.64, 172.58, 71.53, 71.46, 71.43, 71.40, 71.39, 45.22, 45.20, 45.17, 45.13, 45.10, 44.72, 18.09, 18.07, 18.04, 18.03, 17.94, 17.89, 16.53, 13.20, 13.09, 13.07, 12.89, 12.79, 12.76, 12.60, 12.30. DSC Tₘ=161° C., 176° C., T_{c}=133° C., T_{g}=10° C. TGA T_{d, 5%}=289° C.

### Synthesis of Copolymer (cis Content=80%) With Complex B (Pᵣ=0.75 for cis-DMPL)

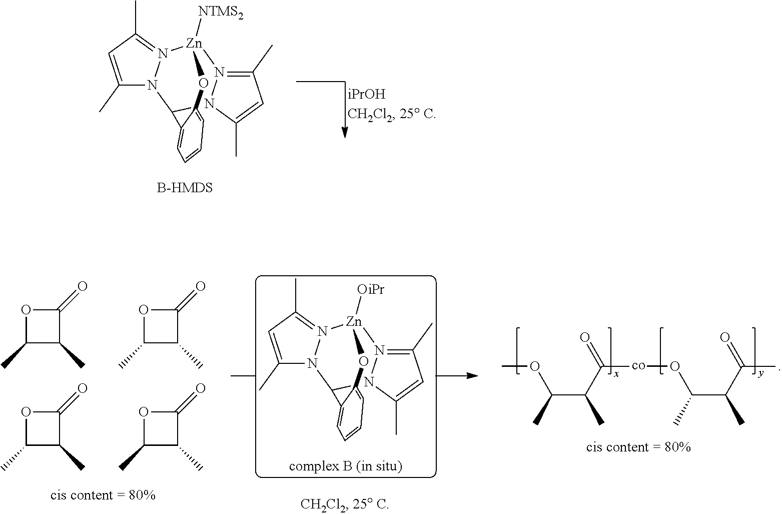

To a solution of cis monomer (0.400 mL, 4.00 mmol, 2000 equiv) and trans monomer (0.100 mL, 1.00 mmol, 500 equiv) in CH₂Cl₂ (0.52 mL) was added a solution of complex B (prepared as described above, 0.200 mL, 0.01 M in CH₂Cl₂, 2.00 μmol, 1 equiv) at 25° C., and the resulting mixture was set at 25° C. for 24 h. The reaction mixture was then removed from glovebox, precipitated from hexane/diethyl ether (2:1 v/v, 300 mL) and dried under vacuum to give the polymer as white foam (0.432 g, 86% yield). GPC Mₙ=128 kDa, Ð=1.05. ¹H NMR (400 MHZ, CDCl₃) δ 5.17-5.07 (m, 1 H), 2.71-2.53 (m, 1 H), 1.24-1.12 (m, 6 H). ¹³C NMR (126 MHz, CDCl₃) δ 172.80, 172.74, 172.72, 172.69, 172.66, 172.60, 71.64, 71.54, 71.44, 71.41, 71.29, 45.22, 45.11, 44.73, 44.64, 44.57, 18.09, 18.06, 18.04, 17.96, 17.90, 16.54, 13.23, 13.11, 12.81, 12.61, 12.36. DSC Tₘ=144° C., 160° C., T_{c}=109° C., T_{g}=9° C. TGA T_{d, 5%}=284° C.

### Synthesis of Copolymer (cis Content=70%) With Complex B (Pᵣ=0.75 for cis-DMPL)

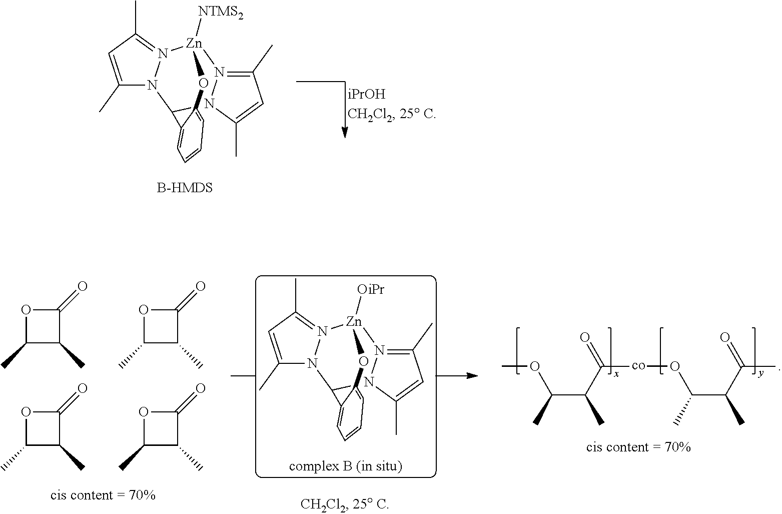

To a solution of cis monomer (0.350 mL, 3.50 mmol, 1500 equiv) and trans monomer (0.150 mL, 1.50 mmol, 1000 equiv) in CH₂Cl₂ (0.55 mL) was added a solution of complex B (prepared as described above, 0.200 mL, 0.01 M in CH₂Cl₂, 2.00 μmol, 1 equiv) at 25° C., and the resulting mixture was set at 25° C. for 24 h. The reaction mixture was then removed from glovebox, precipitated from hexane/diethyl ether (2:1 v/v, 300 mL) and dried under vacuum to give the polymer as white foam (0.396 g, 79% yield). GPC Mₙ=147 kDa, Ð=1.03. ¹H NMR (400 MHZ, CDCl₃) δ 5.17-5.07 (m, 1 H), 2.72-2.55 (m, 1 H), 1.24-1.12 (m, 6 H). ¹³C NMR (126 MHz, CDCl₃) δ 172.80, 172.74, 172.72, 172.68, 172.59, 172.46, 172.35, 71.64, 71.54, 71.41, 71.39, 71.28, 45.22, 45.12, 44.73, 44.57, 44.19, 18.09, 18.04, 17.96, 17.90, 17.79, 16.73, 16.54, 13.22, 13.10, 12.91, 12.78, 12.61, 12.36. DSC Tₘ=125° C., 139° C., T_{c}=78° C., T_{g}=9° C. TGA T_{d, 5%}=276° C.

### Depolymerization of PHMB Copolymers

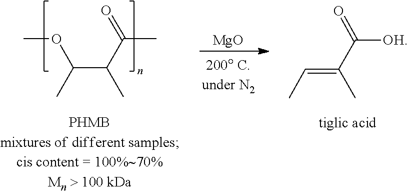

To a flask equipped with a stir bar was added powdered, flame-dried MgO (1.1 mg, 1.0 wt. %) and mixtures of various PHMB samples (0.110 g, cis content=100%˜70%, Mₙ>100 kDa). The flask was connected to a vacuum transfer tube wrapped with heating tape, and a receiving flask at the end (see FIG. 27). The flask was merged into a heated oil bath and the solid mixture was stirred at 200° C. under N₂ for 1 h. After that, the entire setup was connected to vacuum and the product, tiglic acid, was distilled into the receiving flask cooled with liquid N₂ bath. When the distillation was finished, the remaining tiglic acid in the vacuum transfer tube was further washed into the receiving flask with DCM, and the solvent was removed on a rotary evaporator at 30° C. The resulting solid was further dried under vacuum to give tiglic acid as colorless crystals (0.104 g, 93% yield). ¹H NMR (400 MHZ, CDCl₃) δ 12.39 (s, 1 H), 6.93 (q, J=6.6 Hz, 1 H), 1.76-1.73 (m, 6 H). ¹³C NMR (100 MHZ, CDCl₃) δ 173.89, 140.10, 128.21, 14.73, 11.76.

Calculating syndiotacticity of cis-PHMB from quant. ¹³C NMR. Taking cis-PHMB, Pᵣ=0.75 (FIG. 19) as an example: The three sets of peaks around 71 ppm in ¹³C NMR were corresponding to the rr-triad, rm- (and mr-) triad and mm-triad of C^{a} in PHMB. For chain end control: Pᵣᵣ=Pᵣ²=0.56, Pₘₘ=(1−Pᵣ)²=0.07, Pᵣₘ=Pₘᵣ=Pᵣ×(1−Pᵣ), Pᵣₘ+Pₘᵣ=0.37. So, Pᵣ=0.75 is obtained. By applying Bernoulli test, B=4×Pᵣᵣ×Pₘₘ/(Pᵣₘ+Pₘᵣ)²=1.14 which is close to 1, confirming the chain-end control in the polymerization mechanism. The calculated Pᵣ value also matched with the integration of r diad and m diad around 45 ppm, which corresponds to C^{b} in PHMB.

Crystallographic data for B-HMDS. Table 7 shows crystal data and structure refinement for B-HMDS. Table 8 shows atomic coordinates (×10⁴) and equivalent isotropic displacement parameters (Å²×10³). Table 9 shows bond lengths [Å] (and angles [°]) for B-HMDS. Table 10 shows anisotropic displacement parameters (Å²×10³) for B-HMDS. The anisotropic displacement factor exponent takes the form: −2π²[h² a*²U¹¹+ . . . +2 h k a*b*U¹²]. Table 11 shows hydrogen coordinates (×10⁴) and isotropic displacement parameters (Å²×10³) for B-HMDS.

TABLE 7

| Identification code | B-HMDS | | | |
|---|---|---|---|---|
| Empirical formula | C₂₃H₃₇N₅OSi₂Zn | | | |
| Formula weight | 521.12 | | | |
| Temperature | | 99.9(7) | K | |
| Wavelength | | 1.54184 | Å | |
| Crystal system | Orthorhombic | | | |
| Space group | P b c a | | | |
| Unit cell dimensions | a = | 15.55670(10) Å | α = | 90° |
| | b = | 13.86080(10) Å | β = | 90° |
| | c = | 24.8305(2) Å | γ = | 90° |
| Volume | | 5354.16(7) | Å³ | |
| Z | 8 | | | |
| Density (calculated) | | 1.293 | Mg/m³ | |
| Absorption coefficient | | 2.315 | mm⁻¹ | |

| | |
|---|---|
| F(000) | 2208 |
| Crystal size | 0.09 × 0.05 × 0.03 mm³ |
| Theta range for data collection | 3.560 to 78.074° |
| Index ranges | −19 <= h <= 19, −17 <= k <= 17, |
| | −31 <= l <= 31 |
| Reflections collected | 35658 |
| Independent reflections | 5678 [R(int) = 0.0340] |
| Completeness to theta = 67.684° | 100.0% |
| Absorption correction | Gaussian |
| Max. and min. transmission | 1.000 and 0.891 |
| Refinement method | Full-matrix least-squares on F² |
| Data/restraints/parameters | 5678/0/299 |
| Goodness-of-fit on F² | 1.042 |
| Final R indices [I > 2sigma(I)] | R1 = 0.0307, wR2 = 0.0755 |
| R indices (all data) | R1 = 0.0350, wR2 = 0.0777 |
| Extinction coefficient | n/a |
| Largest diff. peak and hole | 0.292 and −0.339e · Å⁻³ |

TABLE 8

| | x | y | z | U(eq) |
|---|---|---|---|---|
| Zn | 5607(1) | 2982(1) | 3692(1) | 17(1) |
| Si(1) | 5483(1) | 2028(1) | 4832(1) | 21(1) |
| Si(2) | 6536(1) | 3836(1) | 4666(1) | 19(1) |
| O | 6081(1) | 3996(1) | 3242(1) | 23(1) |
| N(1) | 5533(1) | 1860(1) | 2674(1) | 19(1) |
| N(2) | 5917(1) | 1862(1) | 3169(1) | 20(1) |
| N(3) | 4290(1) | 2845(1) | 2835(1) | 20(1) |
| N(4) | 4382(1) | 3027(1) | 3372(1) | 21(1) |
| N(5) | 5821(1) | 2978(1) | 4451(1) | 20(1) |
| C(1) | 6009(1) | 4157(1) | 2723(1) | 20(1) |
| C(2) | 6426(1) | 4984(1) | 2514(1) | 25(1) |
| C(3) | 6412(1) | 5221(1) | 1973(1) | 29(1) |
| C(4) | 5978(1) | 4640(1) | 1610(1) | 29(1) |
| C(5) | 5549(1) | 3836(1) | 1802(1) | 25(1) |
| C(6) | 5549(1) | 3580(1) | 2350(1) | 20(1) |
| C(7) | 5031(1) | 2683(1) | 2485(1) | 20(1) |
| C(8) | 5781(1) | 1082(1) | 2380(1) | 21(1) |
| C(9) | 6332(1) | 562(1) | 2700(1) | 25(1) |
| C(10) | 6401(1) | 1070(1) | 3186(1) | 24(1) |
| C(11) | 6911(1) | 829(2) | 3676(1) | 36(1) |
| C(12) | 5492(1) | 902(1) | 1815(1) | 28(1) |
| C(13) | 3453(1) | 2944(1) | 2681(1) | 24(1) |
| C(14) | 3000(1) | 3187(1) | 3135(1) | 27(1) |
| C(15) | 3599(1) | 3228(1) | 3557(1) | 24(1) |
| C(16) | 3464(1) | 3461(2) | 4139(1) | 34(1) |
| C(17) | 3166(1) | 2826(2) | 2112(1) | 33(1) |
| C(18) | 4823(1) | 2392(2) | 5429(1) | 31(1) |
| C(19) | 4791(1) | 1202(1) | 4421(1) | 30(1) |
| C(20) | 6403(1) | 1287(1) | 5107(1) | 29(1) |
| C(21) | 6683(1) | 3868(2) | 5419(1) | 33(1) |
| C(22) | 7623(1) | 3590(1) | 4372(1) | 31(1) |
| C(23) | 6178(1) | 5074(1) | 4465(1) | 26(1) |

TABLE 9

| | |
|---|---|
| Zn—O | 1.9402(12) |
| Zn—N(2) | 2.0793(14) |
| Zn—N(4) | 2.0658(13) |
| Zn—N(5) | 1.9149(13) |
| Si(1)—N(5) | 1.7039(14) |
| Si(1)—C(18) | 1.8726(19) |
| Si(1)—C(19) | 1.8741(18) |
| Si(1)—C(20) | 1.8889(19) |
| Si(2)—N(5) | 1.7135(14) |
| Si(2)—C(21) | 1.8851(18) |
| Si(2)—C(22) | 1.8719(18) |
| Si(2)—C(23) | 1.8724(18) |
| O—C(1) | 1.3123(18) |
| N(1)—N(2) | 1.3674(18) |
| N(1)—C(7) | 1.459(2) |
| N(1)—C(8) | 1.358(2) |
| N(2)—C(10) | 1.333(2) |
| N(3)—N(4) | 1.3646(19) |
| N(3)—C(7) | 1.461(2) |
| N(3)—C(13) | 1.363(2) |
| N(4)—C(15) | 1.331(2) |
| C(1)—C(2) | 1.416(2) |
| C(1)—C(6) | 1.417(2) |
| C(2)—H(2) | 0.9500 |
| C(2)—C(3) | 1.383(2) |
| C(3)—H(3) | 0.9500 |
| C(3)—C(4) | 1.385(3) |
| C(4)—H(4) | 0.9500 |
| C(4)—C(5) | 1.384(3) |
| C(5)—H(5) | 0.9500 |
| C(5)—C(6) | 1.406(2) |
| C(6)—C(7) | 1.519(2) |
| C(7)—H(7) | 1.0000 |
| C(8)—C(9) | 1.372(2) |
| C(8)—C(12) | 1.494(2) |
| C(9)—H(9) | 0.9500 |
| C(9)—C(10) | 1.401(2) |
| C(10)—C(11) | 1.492(2) |
| C(11)—H(11A) | 0.9800 |
| C(11)—H(11B) | 0.9800 |
| C(11)—H(11C) | 0.9800 |
| C(12)—H(12A) | 0.9800 |
| C(12)—H(12B) | 0.9800 |
| C(12)—H(12C) | 0.9800 |
| C(13)—C(14) | 1.371(3) |
| C(13)—C(17) | 1.493(2) |
| C(14)—H(14) | 0.9500 |
| C(14)—C(15) | 1.403(3) |
| C(15)—C(16) | 1.495(2) |
| C(16)—H(16A) | 0.9800 |
| C(16)—H(16B) | 0.9800 |
| C(16)—H(16C) | 0.9800 |
| C(17)—H(17A) | 0.9800 |
| C(17)—H(17B) | 0.9800 |
| C(17)—H(17C) | 0.9800 |
| C(18)—H(18A) | 0.9800 |
| C(18)—H(18B) | 0.9800 |
| C(18)—H(18C) | 0.9800 |
| C(19)—H(19A) | 0.9800 |
| C(19)—H(19B) | 0.9800 |
| C(19)—H(19C) | 0.9800 |
| C(20)—H(20A) | 0.9800 |
| C(20)—H(20B) | 0.9800 |
| C(20)—H(20C) | 0.9800 |
| C(21)—H(21A) | 0.9800 |
| C(21)—H(21B) | 0.9800 |
| C(21)—H(21C) | 0.9800 |
| C(22)—H(22A) | 0.9800 |
| C(22)—H(22B) | 0.9800 |
| C(22)—H(22C) | 0.9800 |
| C(23)—H(23A) | 0.9800 |
| C(23)—H(23B) | 0.9800 |
| C(23)—H(23C) | 0.9800 |
| O—Zn—N(2) | 95.35(5) |
| O—Zn—N(4) | 96.19(5) |
| N(4)—Zn—N(2) | 89.81(5) |
| N(5)—Zn—O | 120.19(5) |
| N(5)—Zn—N(2) | 124.89(6) |
| N(5)—Zn—N(4) | 122.59(6) |
| N(5)—Si(1)—C(18) | 113.55(8) |
| N(5)—Si(1)—C(19) | 110.35(7) |
| N(5)—Si(1)—C(20) | 112.79(8) |
| C(18)—Si(1)—C(19) | 106.28(9) |
| C(18)—Si(1)—C(20) | 105.99(9) |
| C(19)—Si(1)—C(20) | 107.45(9) |
| N(5)—Si(2)—C(21) | 113.77(8) |
| N(5)—Si(2)—C(22) | 109.84(8) |
| N(5)—Si(2)—C(23) | 111.10(8) |
| C(22)—Si(2)—C(21) | 106.36(9) |
| C(22)—Si(2)—C(23) | 109.43(8) |
| C(23)—Si(2)—C(21) | 106.14(9) |
| C(1)—O—Zn | 130.96(11) |
| N(2)—N(1)—C(7) | 121.43(13) |
| C(8)—N(1)—N(2) | 111.10(13) |
| C(8)—N(1)—C(7) | 126.89(13) |
| N(1)—N(2)—Zn | 117.47(10) |
| C(10)—N(2)—Zn | 136.64(11) |
| C(10)—N(2)—N(1) | 105.86(13) |
| N(4)—N(3)—C(7) | 121.78(13) |
| C(13)—N(3)—N(4) | 110.83(14) |
| C(13)—N(3)—C(7) | 127.03(14) |
| N(3)—N(4)—Zn | 117.82(10) |
| C(15)—N(4)—Zn | 135.88(12) |
| C(15)—N(4)—N(3) | 106.25(13) |
| Si(1)—N(5)—Zn | 119.67(8) |
| Si(1)—N(5)—Si(2) | 124.36(8) |
| Si(2)—N(5)—Zn | 114.61(7) |
| O—C(1)—C(2) | 117.26(15) |
| O—C(1)—C(6) | 126.07(15) |
| C(2)—C(1)—C(6) | 116.67(14) |
| C(1)—C(2)—H(2) | 118.6 |
| C(3)—C(2)—C(1) | 122.76(17) |
| C(3)—C(2)—H(2) | 118.6 |
| C(2)—C(3)—H(3) | 119.9 |
| C(2)—C(3)—C(4) | 120.15(17) |
| C(4)—C(3)—H(3) | 119.9 |
| C(3)—C(4)—H(4) | 120.7 |
| C(5)—C(4)—C(3) | 118.55(15) |
| C(5)—C(4)—H(4) | 120.7 |
| C(4)—C(5)—H(5) | 118.7 |
| C(4)—C(5)—C(6) | 122.54(16) |
| C(6)—C(5)—H(5) | 118.7 |
| C(1)—C(6)—C(7) | 125.83(14) |
| C(5)—C(6)—C(1) | 119.32(15) |
| C(5)—C(6)—C(7) | 114.85(15) |
| N(1)—C(7)—N(3) | 110.59(13) |
| N(1)—C(7)—C(6) | 115.22(13) |
| N(1)—C(7)—H(7) | 104.9 |
| N(3)—C(7)—C(6) | 115.09(13) |
| N(3)—C(7)—H(7) | 104.9 |
| C(6)—C(7)—H(7) | 104.9 |
| N(1)—C(8)—C(9) | 106.51(14) |
| N(1)—C(8)—C(12) | 123.45(15) |
| C(9)—C(8)—C(12) | 130.02(15) |
| C(8)—C(9)—H(9) | 126.8 |
| C(8)—C(9)—C(10) | 106.41(15) |
| C(10)—C(9)—H(9) | 126.8 |
| N(2)—C(10)—C(9) | 110.11(14) |
| N(2)—C(10)—C(11) | 120.71(16) |
| C(9)—C(10)—C(11) | 129.17(16) |
| C(10)—C(11)—H(11A) | 109.5 |
| C(10)—C(11)—H(11B) | 109.5 |
| C(10)—C(11)—H(11C) | 109.5 |
| H(11A)—C(11)—H(11B) | 109.5 |
| H(11A)—C(11)—H(11C) | 109.5 |
| H(11B)—C(11)—H(11C) | 109.5 |
| C(8)—C(12)—H(12A) | 109.5 |
| C(8)—C(12)—H(12B) | 109.5 |
| C(8)—C(12)—H(12C) | 109.5 |
| H(12A)—C(12)—H(12B) | 109.5 |
| H(12A)—C(12)—H(12C) | 109.5 |
| H(12B)—C(12)—H(12C) | 109.5 |
| N(3)—C(13)—C(14) | 106.56(15) |
| N(3)—C(13)—C(17) | 122.71(16) |
| C(14)—C(13)—C(17) | 130.70(16) |
| C(13)—C(14)—H(14) | 126.8 |
| C(13)—C(14)—C(15) | 106.41(15) |
| C(15)—C(14)—H(14) | 126.8 |
| N(4)—C(15)—C(14) | 109.95(15) |
| N(4)—C(15)—C(16) | 120.51(16) |
| C(14)—C(15)—C(16) | 129.53(16) |
| C(15)—C(16)—H(16A) | 109.5 |
| C(15)—C(16)—H(16B) | 109.5 |
| C(15)—C(16)—H(16C) | 109.5 |
| H(16A)—C(16)—H(16B) | 109.5 |
| H(16A)—C(16)—H(16C) | 109.5 |
| H(16B)—C(16)—H(16C) | 109.5 |
| C(13)—C(17)—H(17A) | 109.5 |
| C(13)—C(17)—H(17B) | 109.5 |
| C(13)—C(17)—H(17C) | 109.5 |
| H(17A)—C(17)—H(17B) | 109.5 |
| H(17A)—C(17)—H(17C) | 109.5 |
| H(17B)—C(17)—H(17C) | 109.5 |
| Si(1)—C(18)—H(18A) | 109.5 |
| Si(1)—C(18)—H(18B) | 109.5 |
| Si(1)—C(18)—H(18C) | 109.5 |
| H(18A)—C(18)—H(18B) | 109.5 |
| H(18A)—C(18)—H(18C) | 109.5 |
| H(18B)—C(18)—H(18C) | 109.5 |
| Si(1)—C(19)—H(19A) | 109.5 |
| Si(1)—C(19)—H(19B) | 109.5 |
| Si(1)—C(19)—H(19C) | 109.5 |
| H(19A)—C(19)—H(19B) | 109.5 |
| H(19A)—C(19)—H(19C) | 109.5 |
| H(19B)—C(19)—H(19C) | 109.5 |
| Si(1)—C(20)—H(20A) | 109.5 |
| Si(1)—C(20)—H(20B) | 109.5 |
| Si(1)—C(20)—H(20C) | 109.5 |
| H(20A)—C(20)—H(20B) | 109.5 |
| H(20A)—C(20)—H(20C) | 109.5 |
| H(20B)—C(20)—H(20C) | 109.5 |
| Si(2)—C(21)—H(21A) | 109.5 |
| Si(2)—C(21)—H(21B) | 109.5 |
| Si(2)—C(21)—H(21C) | 109.5 |
| H(21A)—C(21)—H(21B) | 109.5 |
| H(21A)—C(21)—H(21C) | 109.5 |
| H(21B)—C(21)—H(21C) | 109.5 |
| Si(2)—C(22)—H(22A) | 109.5 |
| Si(2)—C(22)—H(22B) | 109.5 |
| Si(2)—C(22)—H(22C) | 109.5 |
| H(22A)—C(22)—H(22B) | 109.5 |
| H(22A)—C(22)—H(22C) | 109.5 |
| H(22B)—C(22)—H(22C) | 109.5 |
| Si(2)—C(23)—H(23A) | 109.5 |
| Si(2)—C(23)—H(23B) | 109.5 |
| Si(2)—C(23)—H(23C) | 109.5 |
| H(23A)—C(23)—H(23B) | 109.5 |
| H(23A)—C(23)—H(23C) | 109.5 |
| H(23B)—C(23)—H(23C) | 109.5 |

TABLE 10

| | U¹¹ | U²² | U³³ | U²³ | U¹³ | U¹² |
|---|---|---|---|---|---|---|
| Zn | 18(1) | 21(1) | 12(1) | 0(1) | −1(1) | 0(1) |
| Si(1) | 23(1) | 24(1) | 15(1) | 3(1) | −1(1) | −7(1) |
| Si(2) | 21(1) | 21(1) | 15(1) | −1(1) | 0(1) | −4(1) |
| O | 28(1) | 29(1) | 13(1) | 2(1) | −1(1) | −5(1) |
| N(1) | 23(1) | 19(1) | 14(1) | −1(1) | −3(1) | 3(1) |
| N(2) | 25(1) | 22(1) | 14(1) | 0(1) | −3(1) | 6(1) |
| N(3) | 19(1) | 23(1) | 18(1) | −2(1) | −5(1) | 2(1) |
| N(4) | 18(1) | 25(1) | 19(1) | −2(1) | −1(1) | 1(1) |
| N(5) | 22(1) | 25(1) | 13(1) | 1(1) | −1(1) | −3(1) |
| C(1) | 21(1) | 22(1) | 16(1) | 2(1) | 1(1) | 3(1) |
| C(2) | 29(1) | 24(1) | 22(1) | 1(1) | 3(1) | −1(1) |
| C(3) | 37(1) | 25(1) | 26(1) | 8(1) | 7(1) | 3(1) |
| C(4) | 40(1) | 31(1) | 17(1) | 8(1) | 2(1) | 10(1) |
| C(5) | 32(1) | 26(1) | 17(1) | 1(1) | −4(1) | 7(1) |
| C(6) | 21(1) | 21(1) | 17(1) | 1(1) | −1(1) | 6(1) |
| C(7) | 22(1) | 22(1) | 15(1) | −2(1) | −4(1) | 4(1) |
| C(8) | 24(1) | 18(1) | 20(1) | −2(1) | 3(1) | −1(1) |
| C(9) | 31(1) | 20(1) | 25(1) | 1(1) | 4(1) | 6(1) |
| C(10) | 28(1) | 23(1) | 21(1) | 4(1) | 2(1) | 6(1) |
| C(11) | 44(1) | 40(1) | 24(1) | 6(1) | −3(1) | 21(1) |
| C(12) | 38(1) | 26(1) | 22(1) | −6(1) | 0(1) | 1(1) |
| C(13) | 21(1) | 19(1) | 32(1) | 1(1) | −8(1) | 1(1) |
| C(14) | 17(1) | 25(1) | 40(1) | 2(1) | −2(1) | 2(1) |
| C(15) | 20(1) | 23(1) | 30(1) | 2(1) | 3(1) | 2(1) |
| C(16) | 27(1) | 42(1) | 32(1) | −3(1) | 9(1) | 4(1) |
| C(17) | 28(1) | 35(1) | 36(1) | −4(1) | −16(1) | 3(1) |
| C(18) | 32(1) | 41(1) | 22(1) | 3(1) | 4(1) | −9(1) |
| C(19) | 33(1) | 30(1) | 27(1) | 4(1) | −5(1) | −12(1) |
| C(20) | 35(1) | 29(1) | 25(1) | 8(1) | −6(1) | −4(1) |
| C(21) | 44(1) | 36(1) | 20(1) | −2(1) | −7(1) | −12(1) |
| C(22) | 22(1) | 30(1) | 42(1) | −1(1) | 3(1) | −3(1) |
| C(23) | 29(1) | 23(1) | 28(1) | −2(1) | 2(1) | −2(1) |

TABLE 11

| | x | y | z | U(eq) |
|---|---|---|---|---|
| H(2) | 6729 | 5393 | 2756 | 30 |
| H(3) | 6701 | 5784 | 1851 | 35 |
| H(4) | 5974 | 4791 | 1237 | 35 |
| H(5) | 5243 | 3442 | 1555 | 30 |
| H(7) | 4780 | 2469 | 2134 | 24 |
| H(9) | 6611 | −25 | 2609 | 30 |
| H(11A) | 7063 | 1424 | 3867 | 54 |
| H(11B) | 7437 | 489 | 3570 | 54 |
| H(11C) | 6570 | 415 | 3914 | 54 |
| H(12A) | 4863 | 876 | 1804 | 42 |
| H(12B) | 5728 | 287 | 1689 | 42 |
| H(12C) | 5695 | 1425 | 1582 | 42 |
| H(14) | 2400 | 3303 | 3159 | 33 |
| H(16A) | 3215 | 2901 | 4322 | 51 |
| H(16B) | 3071 | 4011 | 4171 | 51 |
| H(16C) | 4016 | 3625 | 4305 | 51 |
| H(17A) | 3476 | 3284 | 1882 | 49 |
| H(17B) | 2547 | 2952 | 2087 | 49 |
| H(17C) | 3286 | 2167 | 1991 | 49 |
| H(18A) | 5177 | 2775 | 5676 | 47 |
| H(18B) | 4615 | 1814 | 5615 | 47 |
| H(18C) | 4332 | 2779 | 5308 | 47 |
| H(19A) | 4266 | 1542 | 4314 | 45 |
| H(19B) | 4639 | 634 | 4636 | 45 |
| H(19C) | 5106 | 997 | 4099 | 45 |
| H(20A) | 6920 | 1405 | 4891 | 44 |
| H(20B) | 6254 | 601 | 5091 | 44 |
| H(20C) | 6512 | 1471 | 5482 | 44 |
| H(21A) | 6159 | 4119 | 5588 | 50 |
| H(21B) | 7169 | 4288 | 5509 | 50 |
| H(21C) | 6796 | 3215 | 5552 | 50 |
| H(22A) | 7810 | 2940 | 4476 | 47 |
| H(22B) | 8035 | 4065 | 4508 | 47 |
| H(22C) | 7593 | 3634 | 3979 | 47 |
| H(23A) | 6105 | 5101 | 4073 | 40 |
| H(23B) | 6612 | 5547 | 4576 | 40 |
| H(23C) | 5630 | 5223 | 4641 | 40 |

Tensile Test. The polymer samples were melt-pressed with a dog-bone mold (thickness=0.6 mm, width=3 mm, gauge length=10 mm) with the conditions shown below in Table 12.

TABLE 12

| | Melt-press | Melt-press | Melt-press |
|---|---|---|---|
| Sample name | temperature (° C.) | pressure | time (min) |
| 100% cis, Pᵣ = 0.95 | 200 | 5 metric tons | 10 + 1 + 1 + 1 |
| 100% cis, Pᵣ = 0.75 | 180 | 5 metric tons | 10 + 1 + 1 + 1 |
| 100% cis, Pᵣ = 0.63 | 180 | 5 metric tons | 10 + 1 + 1 + 1 |
| 90% cis, Pᵣ = 0.75 | 170 | 5 metric tons | 10 + 1 + 1 + 1 |
| 80% cis, Pᵣ = 0.75 | 160 | 5 metric tons | 10 + 1 + 1 + 1 |
| 70% cis, Pᵣ = 0.75 | 150 | 5 metric tons | 10 + 1 + 1 + 1 |

The samples were loaded with the mold and put into the preheated Carver press between two stainless steel plates, pressed for 10 min, then quickly released from pressure, and pressurized again for 1 min, then release and repeat the 1 min cycle for two more times. The samples were then slowly cooled down (ca. 1˜2° C./min) to lower than 100° C. under the same pressure before they were removed from Carver press and let further cooled down to room temperature. The samples were measured at least one day after being melt-pressed for consistent results.

The samples were too brittle for adequate measurements, as they constantly broke during removal from the dog-bone mold. Tensile data is shown below: cis-PHMB, Pᵣ=0.95 (Table 13); cis-PHMB, Pᵣ=0.75 (Table 14); cis-PHMB, Pᵣ=0.63 (Table 15); 90% cis PHMB copolymer, Pᵣ=0.75 for cis-DMPL (Table 16); 80% cis PHMB copolymer, Pᵣ=0.75 for cis-DMPL (Table 17); 70% cis PHMB copolymer, Pᵣ=0.75 for cis-DMPL (Table 18).

TABLE 13*

| | Young's Modulus | Stress at Break | Strain at Break |
|---|---|---|---|
| entry | (GPa) | (MPa) | (%) |
| 1 | 0.82 | 24.7 | 4 |

*single data set.

TABLE 14

| | Young's Modulus | Stress at Break | Strain at Break |
|---|---|---|---|
| entry | (GPa) | (MPa) | (%) |
| 1 | 0.76 | 41.8 | 13 |
| 2 | 0.68 | 38.7 | 14 |
| 3 | 0.68 | 31.6 | 8 |
| average | 0.71 | 37.4 | 11 |
| standard | 0.04 | 3.9 | 2 |
| deviation | | | |

TABLE 15

| | Young's Modulus | Stress at Break | Strain at Break |
|---|---|---|---|
| entry | (GPa) | (MPa) | (%) |
| 1 | 0.70 | 32.7 | 14 |
| 2 | 0.62 | 23.1 | 8 |
| 3 | 0.57 | 37.6 | 16 |
| average | 0.63 | 31.1 | 12 |
| standard | 0.04 | 5.3 | 3 |
| deviation | | | |

TABLE 16

| | Young's | Yield | Stress at | Strain at |
|---|---|---|---|---|
| | Modulus | Stress | Break | Break |
| entry | (GPa) | (MPa) | (MPa) | (%) |
| 1 | 0.32 | 24.7 | 28.7 | 314 |
| 2 | 0.35 | 25.5 | 32.2 | 447 |
| 3 | 0.36 | 28.1 | 31.3 | 357 |
| average | 0.34 | 26.1 | 30.7 | 373 |
| standard | 0.01 | 1.3 | 1.3 | 50 |
| deviation | | | | |

TABLE 17

| | Young's | Yield | Stress at | Strain at |
|---|---|---|---|---|
| | Modulus | Stress | Break | Break |
| entry | (GPa) | (MPa) | (MPa) | (%) |
| 1 | 0.102 | 10.1 | 36.6 | 775 |
| 2 | 0.109 | 10.5 | 36.7 | 780 |
| 3 | 0.107 | 10.5 | 36.4 | 793 |
| average | 0.106 | 10.3 | 36.6 | 783 |
| standard | 0.003 | 0.2 | 0.1 | 7 |
| deviation | | | | |

TABLE 18

| | Young's | Yield | Stress at | Strain at |
|---|---|---|---|---|
| | Modulus | Stress | Break | Break |
| entry | (GPa) | (MPa) | (MPa) | (%) |
| 1 | 0.064 | 5.7 | 24.0 | 1043 |
| 2 | 0.058 | 5.5 | 21.8 | 938 |
| 3 | 0.063 | 5.7 | 25.6 | 1079 |
| average | 0.062 | 5.6 | 23.8 | 1020 |
| standard | 0.002 | 0.1 | 1.3 | 55 |
| deviation | | | | |

### Thermal Stability Assessment of 90% cis PHMB Copolymer, Pᵣ=0.75 for cis-DMPL

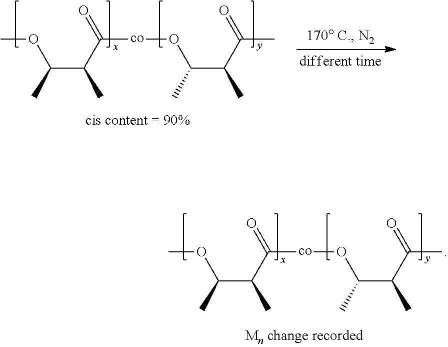

The polymer samples (initial Mₙ=101 kDa, Ð=1.06) were placed in DSC aluminum pan (˜1 mg each) and put in DSC, heated to 170° C. under N₂ for different time (1 min to 2 h). They were then removed from the pan, dissolved in THF, filtered and their molecular weights were measure via GPC analysis. In contrast, R-P3HB was reported to depolymerize rapidly at the same condition (170° C., under N₂) and its Mₙ decreased by ca. 50% within 30 min.

### Kinetic Plot for Copolymerization With Complex B

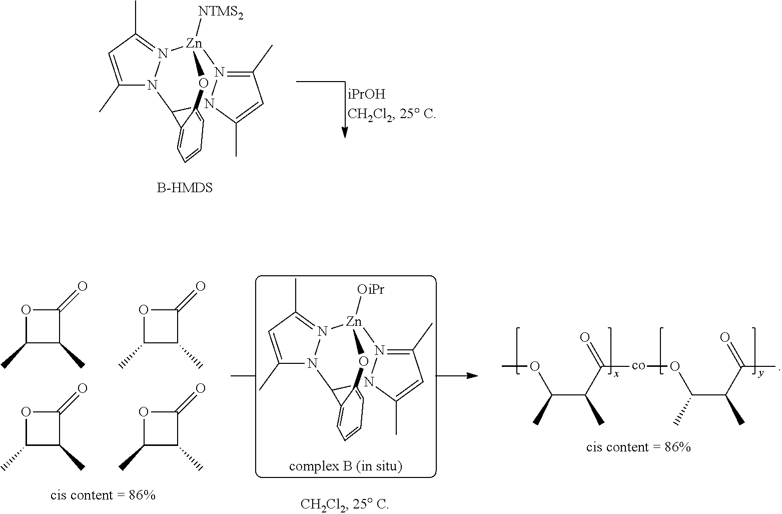

Reaction conditions: mixture of DMPL isomers with cis content=86%, in the presence of complex B, M/I=1400:1, CH₂Cl₂, 2 M, 25° C. Conversions were measured by ¹H NMR of reaction mixture and were calculated from the integrations of peaks of residual monomer versus the sum of polymer and monomers.

### EXAMPLE 3

The following is an example of PDABL compositions of the present disclosure, methods of making same, and uses same.

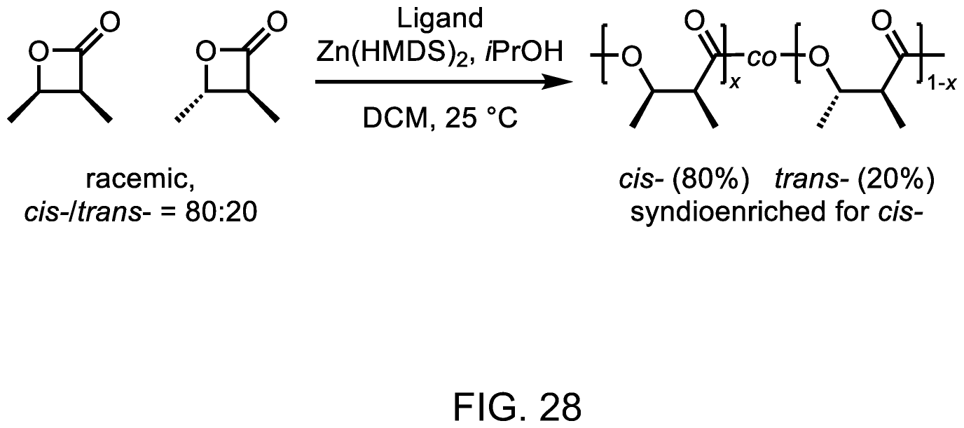

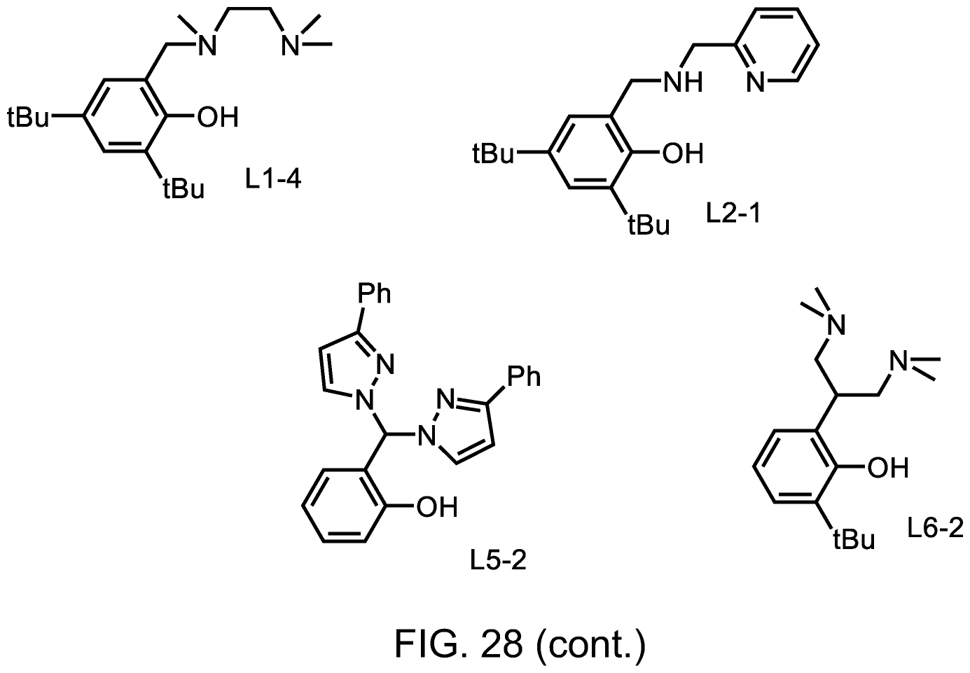

Additional copolymers were made by the methods described in EXAMPLES 1 and 2 using selected catalysts (FIG. 28) and their properties were explored. Selection was mainly based on the fact that these ligands/complexes were expected to give higher syndioselectivities than [(L1-1)ZnOiPr], thus providing more semicrystalline regions in the copolymers. The new copolymers made using an 80% cis (rac-1)/20% trans (rac-2) DMPL feed using the selected ligands shown in FIG. 28 and Table 19 were all semicrystalline with Tm>100° C.

TABLE 19

| | | | | cis | trans | | | | |
|---|---|---|---|---|---|---|---|---|---|
| | cis stereo- | cis/trans | time | conv. | conv. | Mₙ | | Tₘ | T_{c} |
| Ligand | regularity | ratio | (h) | (%) | (%) | (kDa) | M_{w}/Mₙ | (° C.) | (° C.) |
| L1-4^{a} | Pᵣ = 0.67 | 80/20 | 22 | >99 | >99 | 103 | 1.03 | 153 | 82 |
| L2-1^{b} | Pᵣ = 0.68 | 80/20 | 25 | 99 | 68 | 73.1^{c} | 1.05 | 142 | 76 |
| L6-2^{b} | Pᵣ = 0.69 | 80/20 | 24 | 99 | 89 | 115 | 1.05 | 129 | 69 |
| L5-2 | Pₘ = 0.85 | 80/20 | 147 | 95 | 73 | 46.9^{c} | 1.05 | 104 | 75^{d} |

^{a}M/I = 1400, monomer conc. = 4M;
^{b}M/I = 2500, monomer conc. = 4M;
^{c}contains water peak on GPC;
^{d}extremely slow crystallization.

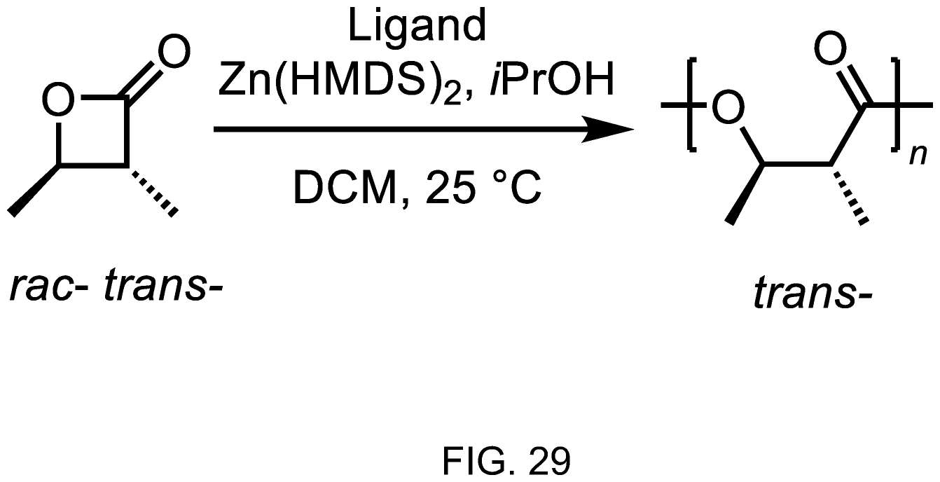

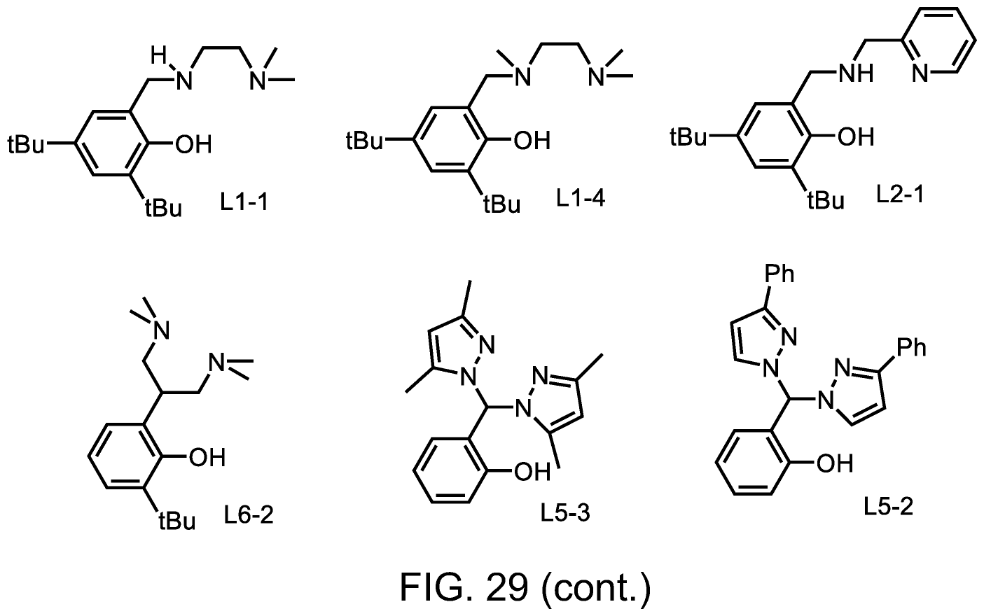

The polymerization of racemic trans-DMPL was also performed with similar zinc complexes bearing selected ligands (FIG. 29). Interestingly, all the catalysts that were previously syndioselective for cis-monomer gave iso-enriched trans-PHMB homopolymers. L5-2 which is isoselective for cis-monomer, on the other hand, gave syndioenriched trans-PHMB (Table 20). Semicrystallinities were observed for some trans-PHMBs possessing high degree of syndio- or isotacticity (with L5-2 or L6-2, respectively).

TABLE 20

| | M/I | time | conv. | Mₙ | | trans stereo- | Tₘ |
|---|---|---|---|---|---|---|---|
| Ligand | ratio | (h) | (%) | (kDa) | M_{w}/Mₙ | regularity | (° C.) |
| L1-1^{a} | 1000 | 3.5 | 96 | 57.3 | 1.03 | Pₘ = 0.72 | amorphous |
| L1-4^{b} | 1400 | 25 | >99 | 153 | 1.24 | Pₘ = 0.67 | amorphous |
| L2-1^{b} | 1400 | 25 | 83 | 128 | 1.15 | Pₘ = 0.62 | amorphous |
| L6-2^{b} | 1400 | 25 | 31 | 67.3 | 1.45 | Pₘ = 0.85 | 161 |
| L5-3^{b} | 2500 | 24 | 85 | 167 | 1.21 | Pₘ = 0.72 | amorphous |
| L5-2^{c} | 1400 | 99 | 30 | 14.8^{d} | 1.66 | Pᵣ = 0.86 | 163 |

^{a}Monomer concentration = 2M;
^{b}monomer concentration = 4M;
^{c}monomer concentration = 5.8M;
^{d}contains water peak on GPC.

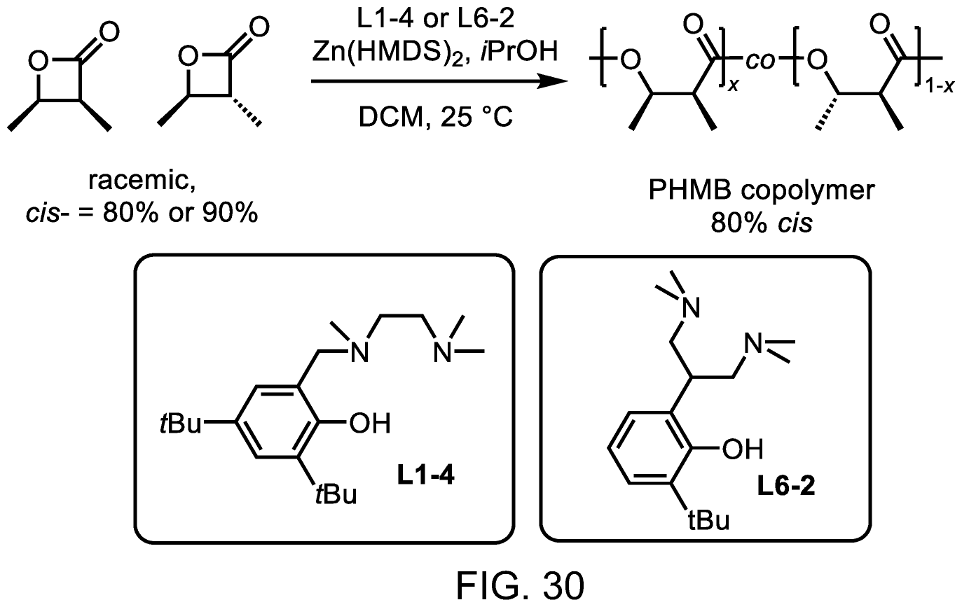

The tensile properties were further measured for selected copolymers (FIG. 30). All the copolymers tested here have >100 kDa molecular weight. Impressively, the 80% copolymer made with [(L1-4)ZnOiPr] is significantly harder (Table 21) than with [(L6-2)ZnOiPr] and two other previously tested ligands (L1-1 and L5-3), although L1-4 doesn't provide the highest tacticities in either cis- or trans-PHMB homopolymers. The 90% cis copolymer made with [(L1-4)ZnOiPr] is also a tough material, with its Young's modulus being even higher than the copolymer with same composition and [(L5-3)ZnOiPr] as catalyst (which gave higher tacticity in both cis- and trans-PHMB homopolymers).

TABLE 21

| | Young's | Yield | Stress at | Strain at |
|---|---|---|---|---|
| | Modulus | Stress | Break | Break |
| sample | (GPa) | (MPa) | (MPa) | (%) |
| 80% cis, with L1-4 | 0.23 | 16 | 29 | 658 |
| 80% cis, with L6-2 | 0.11 | 10 | 32 | 650 |
| 90% cis, with L1-4 | 0.38 | 32 | 38 | 122 |

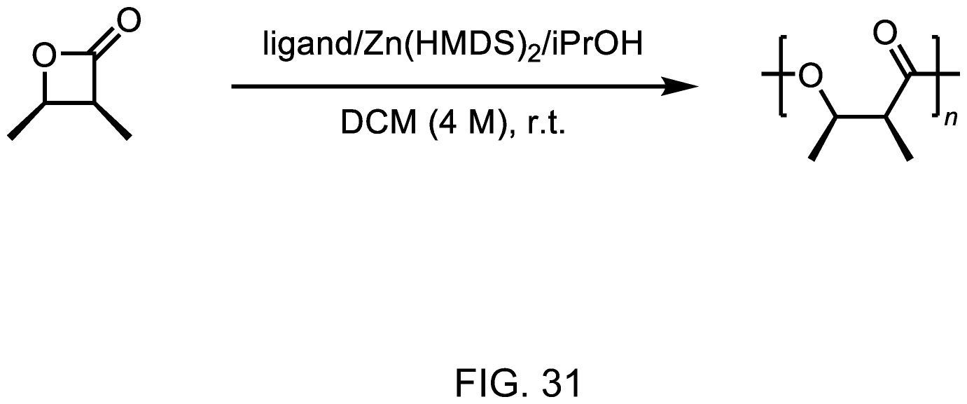

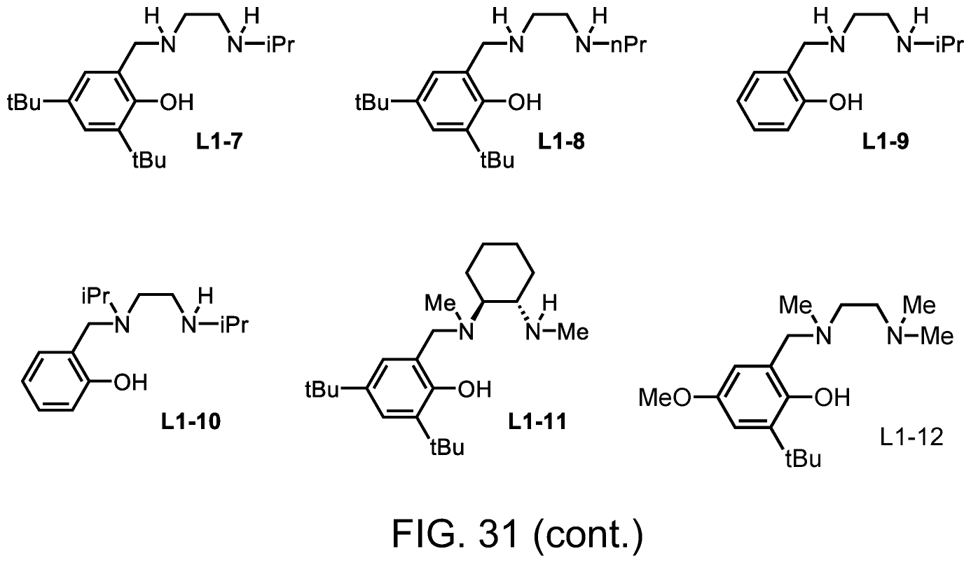

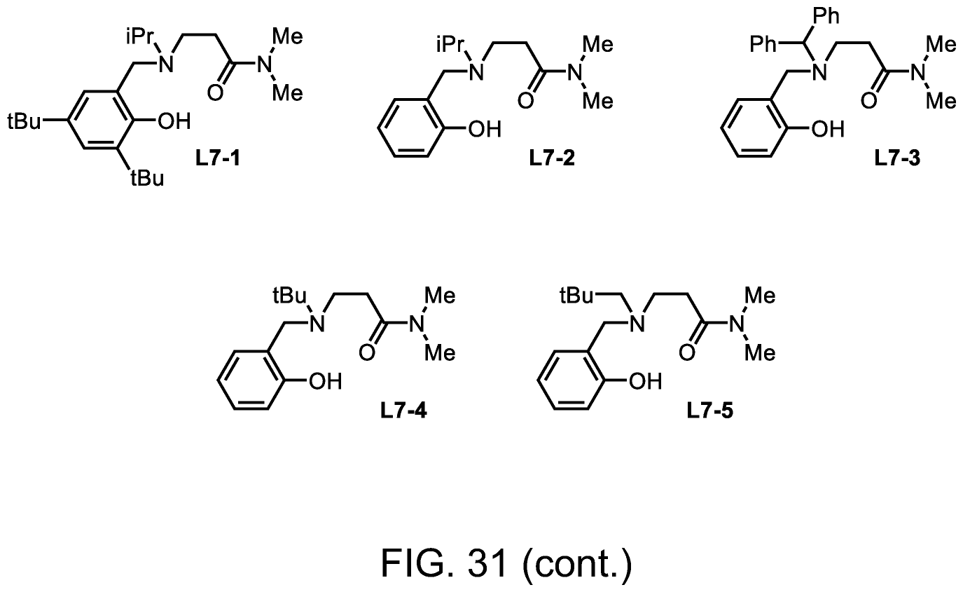

As a continuing goal of catalyst explorations, the ligand library (FIG. 31 and Table 22) was expanded for enhanced reactivity, selectivity and possible hints on the structure-activity relationship. A new class of amide-aminophenol ligand, L7, was found to be moderately active with higher syndioselectivity. In the absence of ligands, polymerization was also observed to proceeded with syndioselectivity, albeit with much diminished reaction rates.

TABLE 22

| | | | | | conv. | | | | |
|---|---|---|---|---|---|---|---|---|---|
| | | | eq | time | (NMR, | Mₙ | M_{w}/ | Micro- | stereo- |
| Lactone | ligand | solvent | lactone^{a} | (h) | %) | (kDa) | Mₙ | structure | regularity |
| rac-1 | L1-7 | DCM | 2500 | 10 | >99 | 143 | 1.05 | cis | Pᵣ = 0.56 |
| | | | | | | | | syndioenriched | |
| rac-1 | L1-8 | DCM | 2500 | 10 | >99 | 131 | 1.04 | cis | Pᵣ = 0.60 |
| | | | | | | | | syndioenriched | |
| rac-1 | L1-9 | DCM | 2500 | 28 | 84 | 127 | 1.02 | cis | Pᵣ = 0.65 |
| | | | | | | | | syndioenriched | |
| rac-1 | L1-10 | DCM | 2500 | 25 | >99 | 136 | 1.08 | cis | Pᵣ = 0.68 |
| | | | | | | | | syndioenriched | |
| rac-1 | L1-11 | DCM | 2500 | 5.5 | 60 | 237 | 1.04 | cis | Pᵣ = 0.68 |
| | | | | | | | | syndioenriched | |
| rac-1 | L1-12 | DCM | 1400 | 24 | >99 | 269 | 1.31 | cis | Pᵣ = 0.66 |
| | | | | | | | | syndioenriched | |
| rac-1 | L7-1 | DCM | 2500 | 25 | >99 | 119 | 1.03 | cis | Pᵣ = 0.65 |
| | | | | | | | | syndioenriched | |
| rac-1 | L7-2 | DCM | 2500 | 18 | >99 | 119 | 1.06 | cis | Pᵣ = 0.69 |
| | | | | | | | | syndioenriched | |
| rac-1 | L7-3 | DCM | 1400 | 26 | 36 | 29.8 | 1.04 | ND | ND |
| rac-1 | L7-4 | DCM | 1400 | 48 | 25 | 28.9 | 1.13 | ND | ND |
| rac-1 | L7-5 | DCM | 1400 | 24 | 47 | 40.2 | 1.22 | cis | Pᵣ = 0.72 |
| | | | | | | | | syndioenriched | |
| rac-1 | none^{b} | DCM | 700^{c} | 24 | 42 | 25.1 | 1.05 | cis | Pᵣ = 0.83 |
| | | | | | | | | syndioenriched | |
| rac-1 | none^{d} | DCM | 700^{c} | 24 | 76 | 56.7 | 1.04 | cis | Pᵣ = 0.74 |
| | | | | | | | | syndioenriched | |

^{a}monomer concentration = 2M unless otherwise noted;
^{b}no ligand, benzhydrol (BH) and MgⁿBu₂ as initiator (2:1 ratio);
^{c}Monomer (lactone) to BH ratio = 700;
^{d}no ligand, benzhydrol (BH) and Zn(HMDS) as initiator (2:1 ratio).

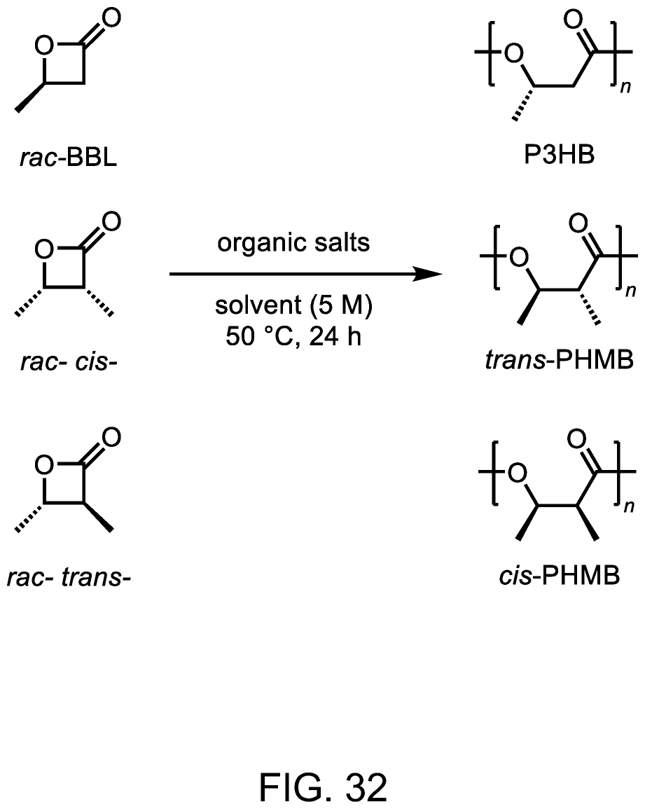

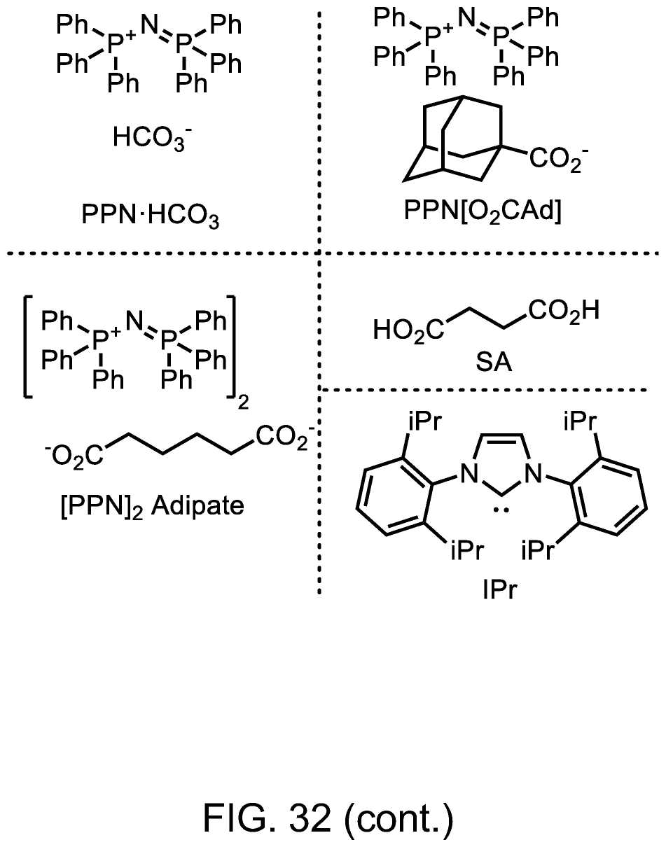

Besides metal complexes as initiators/catalysts, the performance of organocatalysts with inversion of stereochemistry was optimized. This approach makes the most of the trans-enriched DMPL stream, which could come from the remainder of industrial DMPL mixtures after separating cis-enriched monomers. Although PPN salts and N-heterocyclic carbenes (NHC) exhibited appreciable activities for the polymerization of trans- and cis-DMPL in the previous studies, only molecular weight of around 10 kDa were targeted. Here β-butyrolactone (BBL) was also used to probe the activities of the new catalysts before testing on DMPL monomers (FIG. 32). High MW copolymers (close to 100 kDa, Table 23) were obtained with the use of bifunctional initiator and γ-valerolactone (GVL), a polar and green solvent that has similar properties as DMSO and NMP.

TABLE 23

| | | | M/I/C | | | | M_{n,} ₜₕₑₒ | Mₙ | |
|---|---|---|---|---|---|---|---|---|---|
| monomer | initiator | catalyst | ratio | solvent | conv. (%) | product | (kDa) | (kDa) | M_{w}/Mₙ |
| BBL^{a} | PhCO₂H | PPN[HCO₃] | 200/1/0.5 | THF | 77 | P3HB | 13.4 | 12.0 | 1.12 |
| cis- | PhCO₂H | IPr | 500/1/1 | THF | >99 | trans- | 50.1 | 41.8 | 1.09 |
| trans- | PhCO₂H | IPr | 500/1/1 | THF | 64 | cis- | 32.1 | 20.0 | 1.15 |
| BBL | PhCO₂H | IPr | 250/1/0.5 | THF | 53 | P3HB | 11.5 | 13.9 | 1.10 |
| cis- | PhCO₂H | IPr | 250/1/0.5 | THF | 95 | trans- | 23.9 | 25.0 | 1.05 |
| trans- | PhCO₂H | IPr | 250/1/0.5 | THF | 33 | cis- | 8.4 | 9.2 | 1.20 |

| | | | | | | | | |
|---|---|---|---|---|---|---|---|---|
| trans- | PPN[O₂CAd] | 1000/1/— | GVL^{b} | >99 | cis- | 100 | 20.6 | 1.10 |

| | | | | | | | | |
|---|---|---|---|---|---|---|---|---|
| cis- | SA | PPN[HCO₃] | 1000/1/1 | GVL^{b} | >99 | trans- | 100 | 156 (1.04); |
| | | | | | | | | 34.4 (1.56)^{c} |
| trans- | SA | PPN[HCO₃] | 1000/1/1 | GVL^{b} | 93 | cis- | 93 | 80.4 (1.03); |
| | | | | | | | | 17.3 (1.39)^{c} |
| 20% cis- | SA | PPN[HCO₃] | 1000/1/1 | GVL^{b} | >99 (cis-); | 80% cis- | 92 | 110 (1.02); |
| | | | | | 91 (trans-) | | | 16.0 (1.88)^{c} |
| 20% cis- | SA | PPN[HCO₃] | 833/1/2 | GVL^{b, d} | >99 (cis-); | 80% cis- | 81.7 | 70.4 (1.04); |
| | | | | | 98 (trans-) | | | 19.4 (1.42)^{c} |
| 20% cis- | SA | PPN[HCO₃] | 833/1/2 | GVL | >99 (cis-); | 80% cis- | 71.9 | 77.0 (1.04); |
| | | | | | 83 (trans-) | | | 34.9 (1.06) |

| | | | | | | | |
|---|---|---|---|---|---|---|---|
| 20% cis- | [PPN]₂ Adipate | 1250/1/— | GVL | >99 (cis-); | 80% cis- | 76.4 | 94 (1.03) |

52 (trans-)

All the polymers obtained with organocatalysts are atactic.
^{a}monomer concentration = 4M;
^{b}reaction performed at 100° C.;
^{c}the high molecular weight peak is the minor (shoulder) peak;
^{d}reaction performed for 10 hour (h).

Although the present disclosure has been described with respect to one or more particular examples, it will be understood that other examples of the present disclosure may be made without departing from the scope of the present disclosure.

1. A poly(α,β-dialkyl β-lactone) (PDABL) composition comprising one or more polymeric chain(s) and/or one or more oligomeric chain(s) comprising one or more α,β-dialkyl β-lactone (DABL) repeat unit(s) having the following structure: 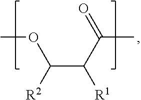
wherein R¹ and R² are each a methyl group, and wherein the polymeric and/or the oligomeric chain(s) is/are not completely trans isotactic.

2. The composition of claim 1, wherein the polymeric and/or the oligomeric chain(s) has/have a weight average molecular weight (Mw) or a number average molecular weight (Mn) of at least about 25 kilodalton (kD) to about 500 kD.

3. The composition of claim 1, wherein the polymeric and/or the oligomeric chain(s) has/have a weight average molecular weight (Mw) or a number average molecular weight (Mn) of at least about 75 kilodalton (kD) to about 300 kD.

4. The composition of claim 1, wherein the polymeric and/or the oligomeric chain(s) has/have a polydispersity index (Mw/Mn) of about 1 to about 10.

5. The composition of claim 1, wherein the polymeric and/or the oligomeric chain(s) has/have a polydispersity index (Mw/Mn) of about 1 to about 1.5.

6. The composition of claim 1, wherein at least a portion of or all of the polymeric and/or the oligomeric chain(s), independently, at least partially or completely, comprise(s) cis and/or trans DABL repeat units. 7 The composition of claim 1, wherein at least a portion of or all of the polymeric and/or the oligomeric chain(s), independently, at least partially or completely, comprise(s) a molar ratio of cis to trans DABL repeat units of about 1:99 to about 99:1.

8. The composition of claim 1, wherein at least a portion of or all of the polymeric and/or the oligomeric chain(s), independently, at least partially or completely, comprise(s) a molar ratio of cis to trans DABL repeat units of about 70:1 to about 99:1.

9. The composition of claim 1, wherein at least a portion of or all of the polymeric and/or the oligomeric chain(s), independently, at least partially or completely, comprise(s) randomly oriented and/or stereoregular DABL diads.

10. The composition of claim 7, wherein the stereoregular DABL diads comprise meso ([m]) and/or racemo ([r]) DABL diads.

11. The composition of claim 1, wherein at least a portion of or all of the polymeric and/or the oligomeric chain(s), independently, at least partially or completely, comprise(s) trans random, cis random, trans meso ([m]), cis meso ([m]), trans racemo ([r]), or cis racemo ([r]) DABL diads, or any combination thereof.

12. The composition of claim 1, wherein at least a portion of the polymeric and/or the oligomeric chain(s), independently, comprise(s) 95 mol % or less of any one of the following: trans random, cis random, trans meso ([m]), cis meso ([m]), trans racemo ([r]), or cis racemo ([r]) DABL diads.

13. The composition of claim 1, wherein at least a portion of or all of the polymeric and/or the oligomeric chain(s), independently, at least partially or completely, is/are atactic, isotactic, isoenriched, syndiotactic, syndioenriched, or any combination thereof.

14. The composition of claim 1, wherein at least a portion of or all of the polymeric and/or the oligomeric chain(s), independently, at least partially or completely, is/are cis atactic, cis isotactic, cis isoenriched, cis syndiotactic, cis syndioenriched, trans atactic, trans isotactic, trans isoenriched, trans syndiotactic, or trans syndioenriched, or any combination thereof.

15. The composition of claim 1, wherein at least a portion of or all of the polymeric and/or the oligomeric chain(s), at least partially or completely, comprise(s) crystalline and/or amorphous domains.

16. The composition of claim 1, wherein at least a portion of or all of the polymeric and/or the oligomeric chain(s), independently, comprise(s) one or more end group(s) independently at each occurrence chosen from hydrogen group (—H), hydroxyl group (—OH), carboxylic acid group (—CO₂H), chloride group (—Cl), azide group (—N₃), acyloxy group (—O₂CR, wherein R is a C₁ to C₂₀ alkyl group or a C₁ to C₂₀ aryl group), and alkoxyl group (—OR, wherein R is a C₁ to C₂₀ alkyl group or a C₁ to C₂₀ aryl group).

17. The composition of claim 1, wherein the PDABL composition comprises one or more homopolymer(s) comprising the DABL repeat unit(s), one or more copolymer(s) comprising the DABL repeat unit(s) and one or more non-DABL repeat unit(s), or any combination thereof.

18. The composition of claim 17, wherein the PDABL composition comprise(s) from about 1 mol % to about 50 mol % of the non-DABL repeat units.

19. The composition of claim 17, wherein the non-DABL repeat unit(s) is/are, independently at each occurrence, chosen from substituted and unsubstituted β-, γ-, δ-, and ω-lactone repeat units, ether group repeat units, carbonate group repeat units, amide group repeat units, and carbamate group repeat units.

20. The composition of claim 17, wherein the non-DABL repeat unit(s) comprise(s) the following structure: 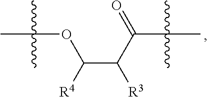
wherein R³ and R⁴ are, independently at each occurrence, a hydrogen group or a C₁, C₂, C₃, C₄, C₅, C₆, C₇, or a C₈ alkyl group, with the proviso that, for each non-DABL repeat unit, at least one of R³ and R⁴ is a hydrogen group.

21. The composition of claim 1, wherein the composition is in the form of a monolith, a film, a fiber, a flake, a pellet, a powder, a granule, a particle, a bead, a bar, a liquid, a solution, an emulsion, or any combination thereof.

22. The composition of claim 1, wherein the composition exhibits or has one or more or all of the following:
a melting temperature (Tm) of about 100° C. to about 250° C.;
an enthalpy of crystallization (ΔHc) of about 10 J/g to about 60 J/g;
a decomposition temperature (Td) of about 240° C. to about 350° C.;
a crystallization temperature (Tc) of about 10° C. to about 200° C.;
a glass transition temperature (Tg) of about −20° C. to about 20° C.;
an elongation at break of about 100% to about 1200%; or
a tensile strength of about 5 MPa to about 50 MPa.

23. The composition of claim 1, wherein at least a portion of or all of the PDABL composition is at least partially or completely biodegradable.

24. A method of forming a poly(α,β-dialkyl β-lactone) (PDABL) composition of claim 1, the method comprising forming a reaction mixture comprising:
dimethyl-β-propiolactone; and
one or more ring opening polymerization (ROP) initiator(s) and one or more ROP catalyst(s), one or more catalyst-initiator(s), one or more precursor(s) thereof, or any combination thereof, wherein the PDABL composition is formed.

25. The method of claim 24, wherein the ROP catalyst-initiator(s) is/are chosen from organic salt(s), carbene(s), aromatic alcohol(s), metal alkoxide(s) and/or aryloxide(s), (multidentate ligand) metal alkoxide complexes, (multidentate ligand) metal aryloxide complex(es), and any combination thereof, and wherein the metal is, independently at each occurrence, chosen from main group metals, transition metals and rare-earth metals.

26. The method of claim 25, wherein:
the organic salt(s) is/are chosen from imidazolium salt(s), aminophosphonium salt(s), diphosphazenium salt(s), ammonium salt(s), and any combination thereof; and/or
the alkoxide(s) and/or aryloxide(s) is/are chosen from C₁-C₂₀ alkoxide(s) and C₁-C₂₀ aryloxide(s).

27. The method of claim 25, wherein the ROP catalyst-initiator(s) is/are chosen from: yttrium (III) tris(isopropoxide) (Y(OⁱPr)₃); benzyl alcohol (BnOH); magnesium (II) benzhydrol (Mg(BH)₂); zinc (II) benzhydrol (Zn(BH)₂); zinc (II) phenoxide isopropoxide ([L]Zn(OⁱPr) or a dimer thereof), wherein L is a phenoxide ligand, independently at each occurrence, chosen from: 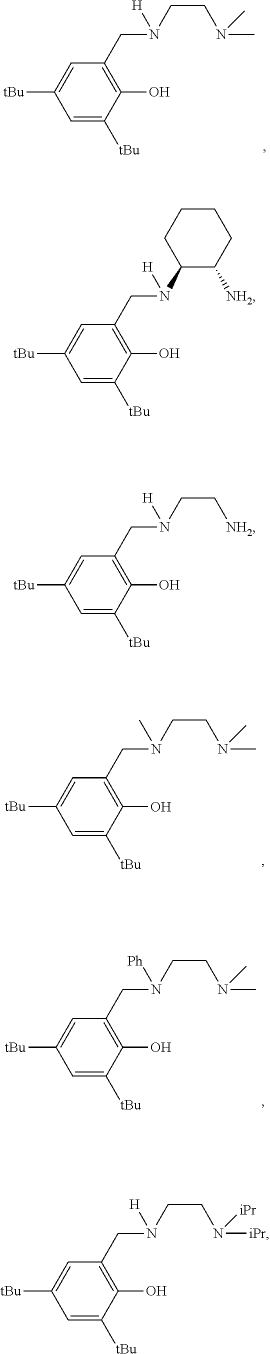 [SMILES: CN(C)CCNCc1cc(C(C)(C)C)cc(C(C)(C)C)c1O ; CC(C)(C)c1cc(CN[C@H]2CCCC[C@@H]2N)c(O)c(C(C)(C)C)c1 ; CC(C)(C)c1cc(CNCCN)c(O)c(C(C)(C)C)c1 ; CN(C)CCN(C)Cc1cc(C(C)(C)C)cc(C(C)(C)C)c1O ; CN(C)CCN(Cc1cc(C(C)(C)C)cc(C(C)(C)C)c1O)c1ccccc1 ; CC(C)N(CCNCc1cc(C(C)(C)C)cc(C(C)(C)C)c1O)C(C)C] 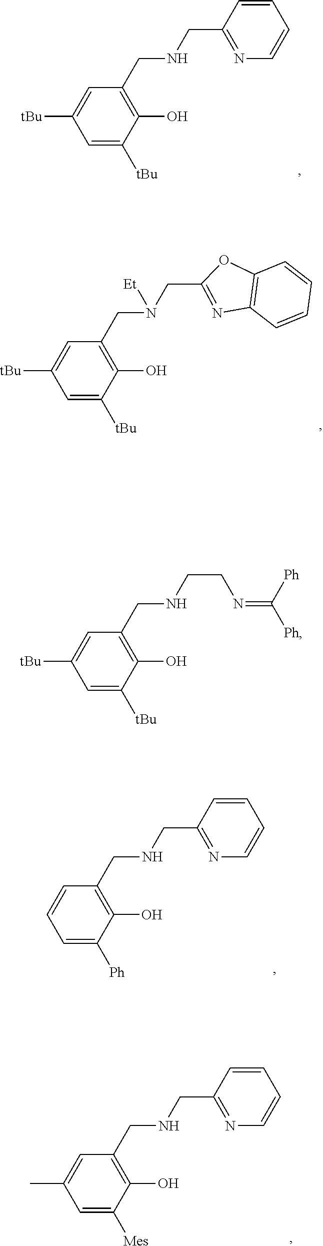 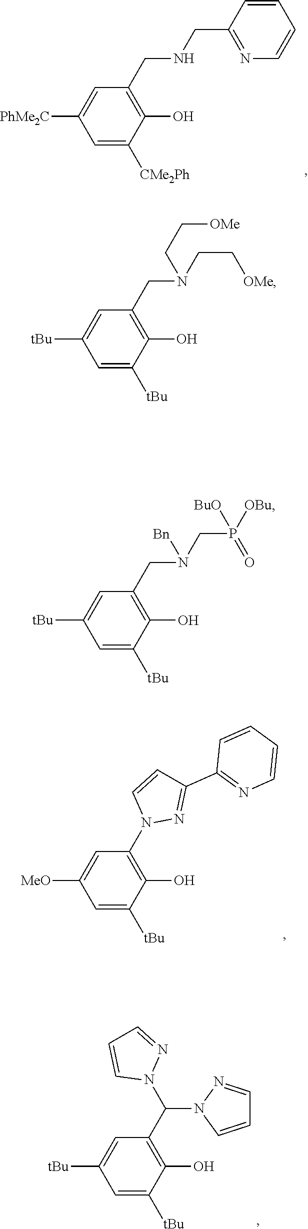 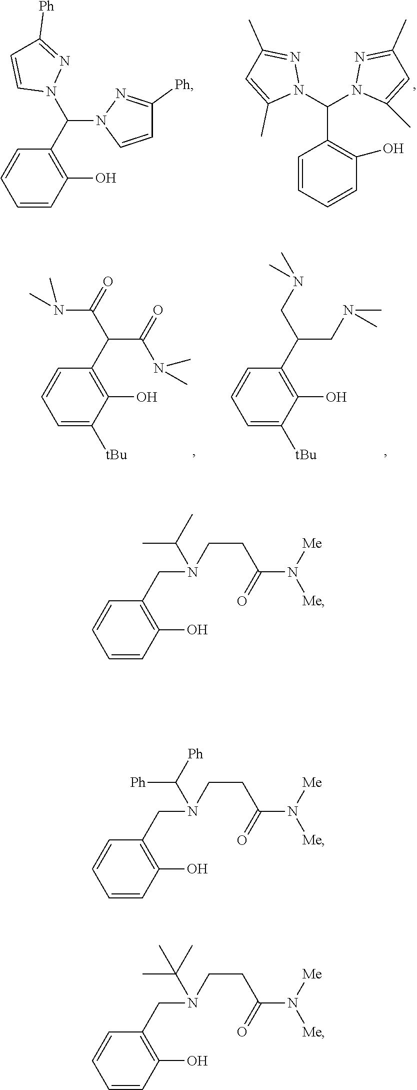 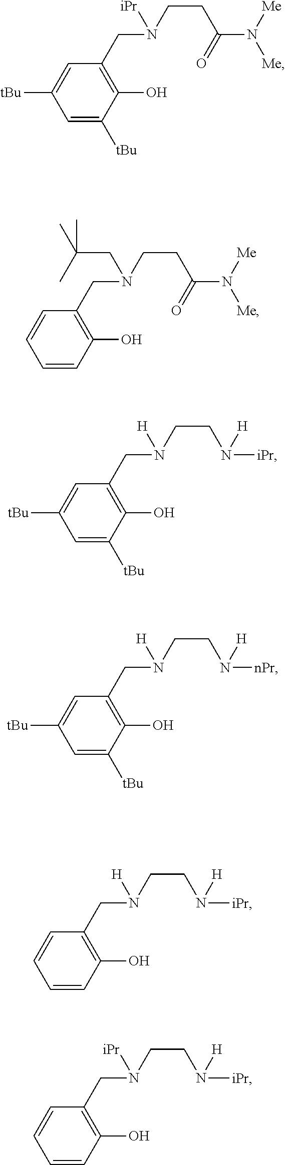 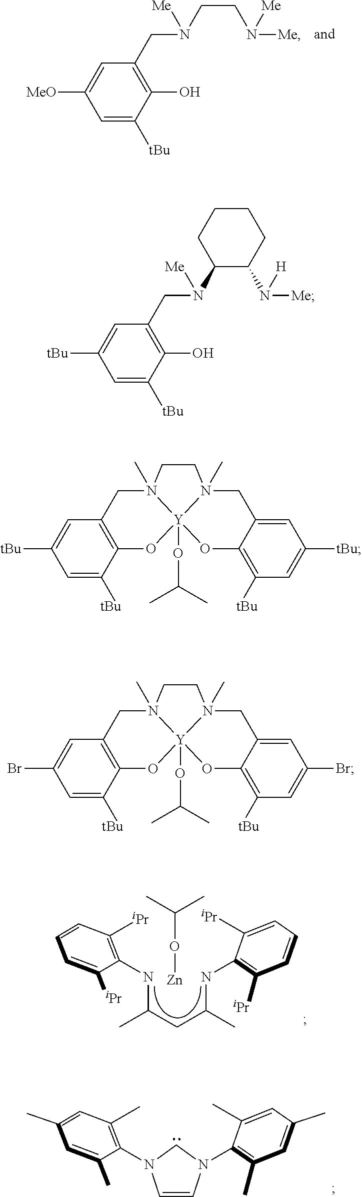 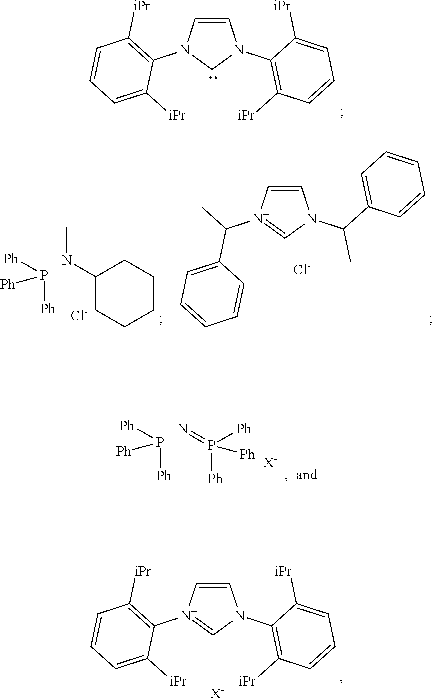
wherein X⁻ is, independently at each occurrence, chosen from RCO₂⁻, Cl⁻, HCO₃⁻, and N₃⁻, and wherein R is, independently at each occurrence. chosen from hydrogen group, aliphatic groups, and aryl groups; and any combination thereof.

28. The method of claim 24, wherein one or more or all of the ROP initiator(s) and/or the ROP catalyst(s), or the ROP catalyst-initiator(s), is/are formed by precursor(s) thereof in situ in the reaction mixture.

29. The method of claim 24, the reaction mixture comprises the one or more ROP catalyst-initiator(s), the precursor(s) thereof, or any combination thereof.

30. The method of claim 24, wherein the reaction mixture further comprises one or more non-DAL monomer(s).

31. The method of claim 30, wherein the non-DAL monomer(s) is/are, independently at each occurrence, chosen from substituted and unsubstituted β-, γ-, δ-, and ω-lactone(s), cyclic ether(s), cyclic carbonate(s), and cyclic carbamate(s).

32. The method of claim 31, wherein the non-DAL monomer(s) is/are, independently at each occurrence, chosen from substituted and unsubstituted β-propiolactone, β-butyrolactone, β-valerolactone, β-caprolactone, lactides, and glycolides.

33. The method of claim 30, wherein the reaction mixture comprises a molar ratio of the DAL(s) to the non-DAL monomer(s) of from about 50:50 to about 100:0.

34. The method of claims 30, wherein the reaction mixture comprises from about 0.01 mol % to about 1 mol % of the ROP initiator(s) and the ROP catalyst(s), the ROP catalyst-initiators(s), the precursor(s) thereof, or any combination thereof, based on the total moles of the DAL(s), the non-DAL monomer(s), and the ROP initiator(s) and the ROP catalyst(s), the ROP catalyst-initiator(s), the precursor(s) thereof, or any combination thereof.

35. The method of claim 24, wherein the reaction mixture further comprises one or more or organic solvent(s), or any combination thereof.

36. The method of claim 35, wherein the organic solvent(s) is/are chosen from polar aprotic solvents, ether solvents, aromatic solvents, chlorinated solvents, lactone solvents, and any combination thereof.

37. The method of claim 24, further comprising forming an article of manufacture by molding, extrusion, blowing, casting, or spinning one or more of the PDABL composition(s).

38. An article of manufacture comprising one or more PDABL composition(s) of claim 1.

39. The article of manufacture of claim 38, wherein the article of manufacture is in the form of a monolith, a coating, a sheet, a film, a fiber, a solid article, a hollow article, a foam, or a composite.

40. The article of manufacture of claim 38, wherein the article of manufacture is a packaging article, a single-use article, a sports article, a biomedical article, an agricultural article, an automotive article, or an electronic article.

41. The article of manufacture of claim 38, wherein:
the packaging article is a film, a wrapping, a sheet, a textile, a net, a bag, a container, a tub, a closure, a cap, a handle, a dispenser, a filler, a protector, a pad, or a fastener;
the single-use article is a bag, a container, a dispenser, a cup, a bottle, a plate, cutlery, or a straw;
the sports article is a fishing line;
the biomedical article is a drug delivery article, a wound closure article, a wound dressing article, a surgical suture, a medical implant, or a tissue engineering construct; or
the agricultural article is a film, a wrapping, a sheet, a textile, a net, a twine, a string, clips, wires, stakes, a bag, a container, a tub, a closure, a cap, a handle, a dispenser, a filler, a protector, a pad, a fastener, a bottle, a lid, a pot, or mulch.

42. The article of manufacture of claim 38, wherein the article of manufacture is biodegradable.

43. A depolymerization method comprising:
forming a depolymerization mixture comprising:
one or more PDABL composition(s) of claim 1, wherein the PDABL composition(s) comprise(s) one or more homopolymer(s) comprising the DABL repeat unit(s); and
one or more depolymerization catalyst(s); and
heating the depolymerization mixture, thereby forming one or more depolymerization product(s).

44. The depolymerization method of claim 43, wherein the depolymerization catalyst(s) is/are chosen from non-metal oxide(s), group II metal oxide(s), group II metal aliphatic carboxylate(s), group II metal aromatic carboxylate(s), aliphatic organic acid(s), aromatic organic acid(s), aliphatic organic base(s), aromatic organic base(s), salts thereof, and any combination thereof.

45. The depolymerization method of claim 44, wherein the group II metal oxide(s) is/are chosen from magnesium oxide, magnesium tiglate, silica gel, sand, p-toluenesulfonic acid, 4-dimethylaminopyridine, and any combination thereof.

46. The depolymerization method of claim 43, wherein the depolymerization mixture comprises from about 1 weight percent (wt. %) to about 20 wt. % of the depolymerization catalyst(s), based on the total weight of the depolymerization mixture.

47. The depolymerization method of claim 43, wherein the depolymerization mixture is heated according to one or more or all of the following:
at a temperature of from about 190° C. to about 220° C.;
for a time of from about 1 hour (h) to about 12 h; or
under inert conditions.

48. The depolymerization method of claim 43, wherein the depolymerization product(s) is tiglic acid.

49. The depolymerization method of claim 43, the method further comprising isolating and, optionally, purifying, the depolymerization product(s).

50. The depolymerization method of claim 43, wherein the depolymerization mixture comprises one or more article(s) of manufacture.
